SEQUENCE LISTING (9 sequences)

SEQ ID NO: 1 - protein, 124 aa, Artificial Sequence
  VH
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSS

SEQ ID NO: 2 - protein, 108 aa, Artificial Sequence
  VL
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 3 - protein, 5 aa, Artificial Sequence
  VHCDR1
SYGMH

SEQ ID NO: 4 - protein, 17 aa, Artificial Sequence
  VHCDR2
VIWFDGTKKYYTDSVKG

SEQ ID NO: 5 - protein, 15 aa, Artificial Sequence
  VHCDR3
DRGIGARRGPYYMDV

SEQ ID NO: 6 - protein, 11 aa, Artificial Sequence
  VLCDR1
RASQSISSYLN

SEQ ID NO: 7 - protein, 7 aa, Artificial Sequence
  VLCDR2
AASSLQS

SEQ ID NO: 8 - protein, 9 aa, Artificial Sequence
  VLCDR3
QQSYSTPLT

SEQ ID NO: 9 - protein, 11 aa, Artificial Sequence
  Abeta 1-11
DAEFRHDSGYE

(19) United States
(12) Patent Application Publication
(10) Pub. No.: US 2015/0315267 A1
(43) Pub. Date: Nov. 5, 2015

(54) A METHOD OF REDUCING BRAIN AMYLOID PLAQUES USING ANTI-AB ANTIBODIES

(71) Applicants: Thierry BUSSIERE, Belmont, MA (US); Paul H. WEINREB, Andover, MA (US); Thomas ENGBER, Acton, MA (US); Kenneth RHODES, Belmont, MA (US); Joseph ARNDT, Peabody, MA (US); Fang QIAN, Southborough, MA (US); Robert W. DUNSTAN, Bedford, MA (US); Shailendra PATEL, Quincy, MA (US); BIOGEN IDEC INTERNATIONAL NEUROSCIENCE GMBH, Schlieren (CH); Neurimmune Holding AG, Schlieren (CH)

(72) Inventors: Thierry Bussiere, Belmont, MA (US); Paul H. Weinreb, Andover, MA (US); Thomas Engber, Acton, MA (US); Kenneth Rhodes, Belmont, MA (US); Joseph Arndt, Peabody, MA (US); Fang Qian, Southborough, MA (US); Robert W. Dunstan, Bedford, MA (US); Shailendra Patel, Quincy, MA (US); Jan Grimm, Dübendorf (CH); Marcel Maier, Zürich (CH)

(21) Appl. No.: 14/650,200

(22) PCT Filed: Dec. 6, 2013
(86) PCT No.: PCT/US13/73700

§ 371 Date: Jun. 5, 2015

Related U.S. Application Data
(60) Provisional application No. 61/734,799, filed on Dec. 7, 2012.
(60) Provisional application No. 61/773,794, filed on Mar. 6, 2013.

(51) Int. Cl. C07K 16/18; A61K 51/10

(52) U.S. Cl. CPC C07K 16/18; A61K 51/1018; C07K 2317/34; C07K 2317/56; C07K 2317/565; C07K 2317/33; C07K 2317/92; C07K 2317/21; C07K 2317/24; A61K 2039/505

(57) ABSTRACT

This disclosure relates to the use of anti-Aβ antibody or antigen-binding fragment thereof to reduce brain amyloid plaques, or minimizes the occurrence of microhemorrhage during chronic dosing of an anti-Aβ antibody or antigen-binding fragment thereof. For example, the disclosure relates to the method of reducing brain amyloid plaques, comprising administering to a subject an anti-Aβ antibody or antigen-binding fragment thereof that binds to the same epitope as BIIB037 antibody, wherein the administration can reduce amyloid plaques in brain without affecting vascular amyloid, and wherein BIIB037 antibody binds to an epitope comprising amino acids 3-6 of Aβ.

### BACKGROUND

1. Field of the Disclosure

This disclosure relates to the method of reducing brain amyloid plaques using anti-Aβ antibodies. Specifically, the disclosure relates to the method of reducing brain amyloid plaques without affecting vascular amyloid, comprising administering to a subject an anti-Aβ antibody or antigen-binding fragment thereof that binds to the same epitope as BIIB037 antibody, or competitively inhibits BIIB037 antibody, wherein the administration reduces amyloid plaques in brain without affecting vascular amyloid, and wherein BIIB037 antibody binds to an epitope comprising amino acids 3-6 of Aβ. Furthermore, the disclosure relates to the method of minimizing the occurrence of microhemorrhage during chronic dosing of an anti-Aβ antibody or antigen-binding fragment thereof.

2. Background of the Disclosure

Amyloid beta (Aβ) peptide is the major component of amyloid plaques in the brain parenchyma (i.e., parenchymal amyloid plaques) of individuals affected by Alzheimer's disease (AD). The formation of the plaques is due to an overproduction of Aβ by amyloid precursor protein (APP) processing, and to Aβ ability to convert under specific conditions from its soluble form into highly ordered fibrillar aggregates. Although neuronal degeneration occurs near the amyloid plaques, some studies have suggested that intermediates such as protofibrils or simple oligomers are also involved in AD pathogenesis and even appear to be the more neurotoxic species in the onset of the pathology. Further, toxic properties of aggregates of different size have been investigated and the obtained results support the hypothesis that different aggregate sizes can induce different degeneration pathways (Di Carlo M., European Biophysics Journal 39(6):877-888 (2010)).

Alzheimer's disease is characterized not only by the presence of parenchymal amyloid plaques and intracellular tangles but also by the presence of amyloid deposits in the vasculature (i.e., vascular amyloid deposits), a condition referred to as cerebral amyloid angiopathy (CAA), also known as congophilic amyloid angiopathy. The CAA observed in both Alzheimer's disease patients and some of the transgenic mouse models is primarily composed of the shorter form of Aβ, Aβ₁₋₄₀, while the majority of amyloid deposits in the parenchyma are composed of Aβ₁₋₄₂. The compact amyloid deposits also contain Aβ₁₋₄₀ (Wilcock et al., Journal of euroinflammation 1(24):1-11 (2004)).

The concept of Aβ immunotherapy as a potential treatment for Alzheimer's disease emerged more than a decade ago, following the demonstration that active immunization with aggregated iv could reduce the development of AD-like pathology or retard the progression of existing pathology in APP transgenic mouse, and also led to improved behavior in those animals (Schenk et Nature 400:173-177 (1999), Janus et al., Neurobiol Aging 21:541-549 (2000), Morgan et al., Nature 408:982-985 (2000)). Translation into a therapeutic approach in human was hampered by the development of adverse events such as meningoencephalitis in a small group of patients (Orgogozo et al., Neurology 61:46-54 (2003)). Additionally, different potential causes have been debated including T-cell activation by the adjuvant, presence of T-cell epitopes on the Aβ peptide, cross-reactivity with physiological APP derivatives and disintegration of blood vessels by reduction of vascular amyloid (Schenk et al., Nat Rev Neurosci 3:824-828).

There is a need to develop a safe method of treatment of neurodegenerative disorders, e.g., Alzheimer's disease, using passive immunization, e.g., anti-Aβ antibodies.

### BRIEF SUMMARY

One embodiment is directed to a method of reducing brain amyloid plaques, comprising administering to a subject an anti-Aβ antibody or antigen-binding fragment thereof that binds to the same epitope as BIIB037 antibody, wherein the administration can reduce amyloid plaques in brain without substantially affecting vascular amyloid, and wherein BIIB037 antibody binds to an epitope comprising amino acids 3-6 of Aβ.

Also disclosed is a method of reducing brain amyloid plaques, comprising administering to a subject an anti-Aβ antibody or antigen-binding fragment thereof that competitively inhibits BIIB037 antibody, wherein the administration can reduce amyloid plaques in brain without substantially affecting vascular amyloid.

Further disclosed is a method of minimizing the occurrence of microhemorrhage during chronic dosing of an anti-Aβ antibody or antigen-binding fragment thereof, comprising administering to a subject an anti-Aβ antibody that binds to the same epitope as BIIB037 antibody, wherein BIIB037 antibody binds to an epitope comprising amino acids 3-6 of Aβ.

Also disclosed is a method of minimizing the occurrence of microhemorrhage during chronic dosing of an anti-Aβ antibody or antigen-binding fragment thereof, comprising administering to a subject an anti-Aβ antibody that competitively inhibits BIIB037 antibody.

Further disclosed is a method of measuring the amount of brain amyloid plaques in a test subject, comprising: (a) measuring the signal generated in the brain of a test subject following administration of an anti-Aβ antibody or antigen-binding fragment thereof that binds to the same conformational epitope as the BIIB037 antibody, wherein the antibody or antigen-binding fragment thereof is labeled with an agent that generates a measurable signal; and (b) comparing the signal generated in the test subject to a signal generated upon administration of the labeled antibody or antigen-binding fragment thereof to one or more control subjects; wherein an increase in the signal generated in the test subject relative to the control subject correlates with an increase in brain amyloid plaques.

Also disclosed is method of treating a neurodegenerative disease characterized by brain amyloid plaques in a patient in need of treatment, comprising: (a) administering an anti-Aβ antibody or antigen-binding fragment thereof that binds to the same conformational epitope as the BIIB037 antibody to a patient in need of neurodegenerative disease treatment, wherein the antibody or antigen-binding fragment thereof is labeled with an agent that generates a measurable signal; (b) assessing the disease state in the patient upon review of a comparison of the signal measured in the patient to the signal measured following administration of the labeled antibody or antigen-binding fragment thereof to one or more control subjects; wherein an increase in the signal generated in the patient relative to the control subject correlates with an increase in brain amyloid plaques; and (c) treating the patient with a therapy appropriate for the patient's disease state.

One embodiment is directed to a method of measuring the amount of brain amyloid plaques in a test subject, comprising: (a) measuring the signal generated in the brain of a test subject following administration of an anti-Aβ antibody or antigen-binding fragment thereof that competitively inhibits the BIIB037 antibody, wherein the antibody or antigen-binding fragment thereof is labeled with an agent that generates a measurable signal; and (b) comparing the signal generated in the test subject to a signal generated upon administration of the labeled antibody or antigen-binding fragment thereof to one or more control subjects; wherein an increase in the signal generated in the test subject relative to the control subject correlates with an increase in brain amyloid plaques.

Further disclosed is a method of treating a neurodegenerative disease characterized by brain amyloid plaques in a patient in need of treatment, comprising: (a) administering an anti-Aβ antibody or antigen-binding fragment thereof that competitively inhibits the BIIB037 antibody to a patient in need of neurodegenerative disease treatment, wherein the antibody or antigen-binding fragment thereof is labeled with an agent that generates a measurable signal; (b) assessing the disease state in the patient upon review of a comparison of the signal measured in the patient to the signal measured following administration of the labeled antibody or antigen-binding fragment thereof to one or more control subjects; wherein an increase in the signal generated in the patient relative to the control subject correlates with an increase in brain amyloid plaques; and (c) treating the patient with a therapy appropriate for the patient's disease state.

In certain embodiments, the control subjects are normal healthy individuals, individuals with neurodegenerative disorders of varying severity, or a combination thereof.

Also disclosed is a method of assessing disease progression in a patient being treated for a neurodegenerative disease characterized by brain amyloid plaques, comprising: (a) administering an anti-Aβ antibody or antigen-binding fragment thereof that binds to the same conformational epitope as the BIIB037 antibody to the patient in need of neurodegenerative disease treatment wherein the antibody or antigen-binding fragment thereof is labeled with an agent that generates a measurable signal, wherein the signal is measured in the patient following the administration; (b) administering the labeled anti-Aβ antibody or antigen-binding fragment thereof at one or more time intervals following the administration of (a), wherein the signal is measured in the patient following the administration; (c) assessing disease progression in the patient based on a change in the signal measured in the patient at the one or more time intervals following administration of (a); wherein an increase in the signal indicates progression of the neurodegenerative disease in the patient.

Further disclosed is a method of assessing disease progression in a patient being treated for a neurodegenerative disease characterized by brain amyloid plaques, comprising: (a) administering an anti-Aβ antibody or antigen-binding fragment thereof that that competitively inhibits the BIIB037 antibody to the patient in need of neurodegenerative disease treatment wherein the antibody or antigen-binding fragment thereof is labeled with an agent that generates a measurable signal, wherein the signal is measured in the patient following the administration; (b) administering the labeled anti-AP antibody or antigen-binding fragment thereof at one or more time intervals following the administration of (a), wherein the signal is measured in the patient following the administration; (c) assessing disease progression in the patient based on a change in the signal measured in the patient at the one or more time intervals following administration of (a); wherein an increase in the signal indicates progression of the neurodegenerative disease in the patient.

Certain embodiments include the method as described herein, further comprising adjusting the patient's treatment based on the level of disease progression.

In some embodiments, the therapy appropriate for the patient's disease state comprises administering an anti-Aβ antibody or antigen-binding fragment thereof that binds to the same conformational epitope as the BIIB037 antibody to the patient in need of the neurodegenerative disease treatment.

Certain embodiments include the method as described herein, wherein the therapy appropriate for the patient's disease state comprises administering an anti-Aβ antibody or antigen-binding fragment thereof that competitively inhibits the BIIB037 antibody to the patient in need of the neurodegenerative disease treatment.

The method of any one of claims of 6 or 8, further comprising:(d) administering the labeled anti-Aβ antibody or antigen-binding fragment thereof at one or more time intervals following the administration of (a), wherein the signal is measured in the patient following the administration; and (e) assessing disease progression in the patient based on a change in the signal measured in the patient at the one or more time intervals following administration of (a); wherein an increase in the signal indicates progression of the neurodegenerative disease in the patient.

In certain embodiments, the agent comprises one or more radioactive ligand(s).

In certain embodiments, the ligand is ¹²⁵I.

In certain embodiments, the signal generated by the agent is measured by single-photon emission computed tomography.

In certain embodiments, the anti-Aβ antibody or antigen-binding fragment thereof comprises a heavy chain variable region (VH) and a light chain variable region (VL), and wherein the VH comprises a complementarity determining region-1 (VHCDR1) amino acid sequence of SEQ ID NO: 3.

In certain embodiments, the anti-Aβ antibody or antigen-binding fragment thereof comprises a VH and a VL, and wherein the VI-1 comprises a complementarity determining region-2 (VHCDR2) amino acid sequence of SEQ ID NO: 4.

In certain embodiments, the anti-Aβ antibody or antigen-binding fragment thereof comprises a VH and a VL, and wherein the VH comprises a complementarity determining region-3 (VHCDR3) amino acid sequence of SEQ ID NO: 5.

In certain embodiments, the anti-Aβ antibody or antigen-binding fragment thereof comprises a VH and a VL, and wherein the VL comprises a complementarity determining region-1 (VLCDR1) amino acid sequence of SEQ ID NO: 6.

In certain embodiments, the anti-Aβ antibody or antigen-binding fragment thereof comprises a VH and a VL, and wherein the VL comprises a complementarity determining region-2 (VLCDR2) amino acid sequence of SEQ ID NO: 7.

In certain embodiments, the anti-Aβ antibody or antigen-binding fragment thereof a VH and a VL, wherein the VL comprises a complementarity determining region-3 (VLCDR3) amino acid sequence of SEQ ID NO: 8.

In certain embodiments, the anti-Aβ antibody or antigen-binding fragment thereof comprises a VH and a VL, and wherein the VH comprises VHCDR1, VHCDR2, and VHCDR3 amino acid sequences of SEQ ID NOs: 3, 4, 5.

In certain embodiments, the anti-Aβ antibody or antigen-binding fragment thereof comprises a VH and a VL, and wherein the VL comprises VLCDR1, VLCDR2, and VLCDR3 amino acid sequences of SEQ ID NOs: 6, 7, 8.

In certain embodiments, the anti-Aβ antibody or antigen-binding fragment thereof comprises a VH and a VL, wherein the VH comprises VHCDR1, VHCDR2, and VHCDR3 amino acid sequences of SEQ ID NOs: 3, 4, 5, and the VL, comprises VLCDR1, VLCDR2, and VLCDR3 amino acid sequences of SEQ ID NOs: 6, 7, 8.

In certain embodiments, the anti-Aβ antibody or antigen-binding fragment thereof comprises a VH and a VL, wherein the VH comprises SEQ ID NO: 1 and the VL comprises SEQ ID NO: 2.

In certain embodiments, the anti-Aβ antibody or antigen-binding fragment thereof comprises the antigen-binding region of BIIB037 antibody.

In certain embodiments, the anti-Aβ antibody or antigen-binding fragment thereof binds selectively to Aβ aggregates.

In certain embodiments, the anti-Aβ antibody or antigen-binding fragment thereof could bind to an Abeta fibril, but does not substantially to an Abeta monomer.

In certain embodiments, the anti-Aβ antibody or antigen-binding fragment thereof can cross the blood-brain barrier in an effective amount to reduce brain amyloid plaques.

In certain embodiments, the anti-Aβ antibody or antigen-binding fragment thereof binds to parenchymal amyloid plaques with a greater affinity than to vascular amyloid.

In certain embodiments, the anti-Aβ antibody or antigen-binding fragment thereof accumulates to a lesser extent in congophilic amyloid angiopathy lesions than on parenchymal amyloid plaques.

In certain embodiments, the reduction of amyloid plaques in brain involves Fc receptor engagement.

In certain embodiments, the anti-Aβ antibody antigen-binding fragment is a single chain Fv fragment (scFv), an F(ab′) fragment, an F(ab) fragment, or an F(ab′)₂ fragment. In one embodiment the antibody is human. In another embodiment the antibody is chimeric.

In certain embodiments, the subject has a neurodegenerative disorder. In certain embodiments, the neurodegenerative disease is selected from the group consisting of Alzheimer's disease, Down's syndrome, mild cognitive impairment, cerebral amyloid angiopathy, vascular dementia, multi-infarct dementia, Parkinson's disease, Dementia with Lewy Bodies, Huntington's disease, Creutzfeldt-Jakob disease, cystic fibrosis, or Gaucher's disease.

Certain embodiments include the method as described herein, for treating or preventing the progression of Alzheimer's disease; for the amelioration of symptoms associated with Alzheimer's disease; for diagnosing or screening a subject for the presence of Alzheimer's disease or for determining a subject's risk for developing Alzheimer's disease.

In certain embodiments, the antibody or antigen-binding fragment thereof is administered by parenteral administration.

In certain embodiments, the antibody or antigen-binding fragment thereof is administered by peripheral administration.

In certain embodiments, the antibody or antigen-binding fragment thereof is administered by parenteral administration.

### BRIEF DESCRIPTION OF THE DRAWINGS/FIGURES

FIG. 1 (A-D): The chimeric BIIB037 (“chBIIB037”) binds selectively to Aβ aggregates. (A) Binding of chBIIB037 or 3D6 to Aβ(1-42) aggregates. (B) Capture of soluble, monomeric Aβ(1-40) by 3D6 immobilized indirectly on an ELISA plate, but not chBIIB037. (C) Preparations of aggregated (lane 2) or monomeric Aβ(1-42) (lane 1) were immunoprecipitated using either BIIB037 or 3D6. (D) Immunostaining of human Alzheimer's disease brain tissue using FITC-conjugated chBIIB037.

FIG. 2 (A-D): Brain penetration of BIIB037 after a single intra-peritoneal administration in Tg2576 transgenic mice. (A) BIIB037 levels were measured in plasma (μg/ml) and brain (ng/g) over 3 weeks after a single dose (30 mg/kg). (B) Plasma Aβ concentrations (pmol/ml) were measured after a single dose of BIIB037 or 3D6 antibody. (C) In vivo binding of BIIB037 to amyloid deposits after a single dose in Tg2576 mice was compared to (D) staining of a consecutive section with a pan-Aβ antibody.

FIG. 3 (A-E): Binding of chBIIB037 to parenchymal amyloid plaques following acute dosing in Tg2576 transgenic mice. Binding of (A) Cy3-labeled chBIIB037 or (C) Cy3-labeled 3D6 to amyloid deposits in the brain was revealed under fluorescent microscope, and (B, D, E) quantified using VISIOPHARM® software. (E) Cy3 staining associated with one or the other amyloid compartment was expressed as percent of total brain area staining, and demonstrated preferential binding of chBIIB037 to parenchymal amyloid vs. vascular amyloid.

FIG. 4 (A-C): Exposure following 6 months of chronic dosing with chBIIB037 in Tg2576 transgenic mice. (A) chBIIB037 concentrations in plasma (μg/ml) were measured 24 hours after the last dose for the 0.3, 1, 3, 10 and 30 mg/kg chBIIB037 treatment groups. chBIIB037 levels above the limit of detection in the PBS group are due to endogenous anti-Aβ antibodies being measured by the assay. (B) chBIIB037 concentrations were also measured in the DEA, (detergent-free) brain fractions for the 0.3, 1, 3, 10 and 30 mg/kg chBIIB037 treatment groups. (C) Correlations of drug concentrations in plasma (open circles) or brain (triangles) with administered dose. Each symbol represents one animal, and the dotted line represents the limit of detection of the assay.

FIG. 5 (A-D): Reduction of amyloid burden following 6 months of chronic dosing with chBIIB037 in Tg2576 transgenic mice. Aβ42 concentration was significantly reduced in both (A) soluble DEA and (C) insoluble guanidine brain fractions for the 3, 10, and 30 mg/kg chBIIB037 treatment groups, compared to the PBS control group. Total brain amyloid load in (B) the cortex and (D) hippocampus was revealed by 6E10 immunohistochemistry and quantified using the VISIOPHARM® software. (Data are represented as mean±SEM; dotted line represents 50% reduction compared to the PBS-treated control group. Differences between treatment and control groups were determined by the Dunn's post-hoc test following Kruskall-Wallis one way analysis of variance based on ranks. Statistically significant difference from vehicle are indicated with asterisk (*); p<0.05).

FIG. 6 (A-D): Binding of chBIIB037 to vascular amyloid following chronic dosing with chBIIB037 in Tg2576 transgenic mice. (A) Thioflavin S (Thio-S) staining of compact amyloid plaques and CAA was revealed under fluorescent microscope, and (B) Visiopharm® software was used to differentiate parenchymal deposits from vascular deposits, and quantified the area occupied for each entity. Vascular amyloid load in (C) cortex and (D) hippocampus was assessed by Thioflavin S staining for the 3, 10, and 30 mg/kg doses of chBIIB037. (E) Differences in the level of microhemorrhage in Tg2576 mice, as defined by Perls staining, after 13 weeks of i.v. treatment of chBIIB037 at 0, 10, 70 and 500 mg/kg, and 500 mg/kg of BIIB037. (Data are represented as mean±SEM; n=18-20 animals per group).

FIG. 7 (A-B): Reduction of amyloid burden following 6 months of chronic dosing with chBIIB037 in Tg2576 transgenic mice. (A) wt chBIIB037, Aglycosylated (Agly) chBIIB037 or 3D6 were dosed at 3 mg/kg via weekly intraperitoneal injection in Tg2576 mice. Brain Aβ₄₂ levels (pmol/g) measured in the insoluble (guanidine) fraction prepared from brain homogenates were compared to the PBS-treated control group. (Differences between treatment and control groups were determined by the Dunn's post-hoc test p<0.01, n=12-14 per group; mean±SEM). (B) Cortical compact amyloid plaque load quantified by Thio-S staining (% stained area) was assessed in wt chBIIB037, Agly chBIIB037, and 3D6 treatment group by quantification of area stained with Thio-S and compared to the PBS control group. (Differences between treatment and control groups were determined by the Dunn's post-hoc test; p<0.01, n=12-14 per group; mean±SEM). Agly chBIIB037 and 3D6 had no effect on the compact amyloid plaque load.

FIG. 8 (A-C): Amyloid load reduction following chronic dosing with chBIIB037 in Tg2576 transgenic mice affects plaques of all sizes. (A,B) Amyloid plaques stained with 6E10 were quantified using VISIOPHARM® software. The size of plaques was defined by area: plaques <125 μm² (#4), 125-250 μm² (#3), 250-500 μm² (#2) and >500 μm² (#1). (C) The plaque numbers in all size ranges were determined in chBIIB037 treatment group and compared to the PBS-treated controls. (t-test, *=p<0.0.005).

FIG. 9 (A-B): Microglia-mediated clearance of amyloid plaques following chronic dosing with chBIIB037 in Tg2576 transgenic mice. (A) Brain sections from either PBS- or chBIIB037-treated mice were immunostained for Aβ (6E10-light gray) and a marker of microglia (Iba1-dark gray). (B) Plaques with circumferences ≧70% surrounded by macrophages were calculated and stratified based on plaque size. Percentage of plaques that are 70% surrounded by microglia compared between the chBIIB037-treated group (clear bars) and the PBS control group (grey bars), for plaques ≧250 μm² (t-test, *=p<0.0.005).

FIG. 10 (A-B): Structure of the BIIB037/AP complex (A) human BIIB037 with Aβ₂₋₉. Aβ₂₋₉ peptide in stick representation with oxygens colored light gray, nitrogens in dark gray, and carbons in white. CDRs contacting the peptide: VHCDR1 (H1), VHCDR2 (H2), VHCDR3 (H3), VLCDR1 (L1), VLCDR2 (L2), and VLCDR3 (L3). Only CDRs contacting the peptide are shown. (B) Superposition of Aβ (3-6) peptide backbones in structures of BIIB037 (white) and W02 (medium gray).

FIG. 11 (A-B): Binding of BIIB037 to mouse Aβ (A) Alignment of human and mouse Aβ (1-42) sequences. (B) Binding of BIIB037 to human and mouse Aβ (1-16) peptides

FIG. 12 (A-C): (A) Overview of single photon emission computed tomography (SPECT) imaging data in Tg2576 (Tg) and WT mouse CNS using [¹²⁵I]chBIIB037; following (B) 7 days and (C) 14 days post intravenous administration of the labeled antibody.

FIG. 13 (A-C): (A) Representative sagittal examples of normalized uptake of [¹²⁵I]chBIIB037 into CNS of Tg2576 (a) and WT (b) mice. (B) Representative day 48 images of the in vivo accumulation of [¹²⁵I]chBIIB037 into CNS of Tg2576 mice. (C) Average normalized brain uptake of [¹²⁵I]chBIIB037 in (a) Ig and (b) WT mouse brain.

FIG. 14 (A-C): In vivo uptake of [¹²⁵I]chBIIB037 in Tg and WT mouse brain versus blood pool. FIG. 14A shows average brain uptake in Tg2576 (L) versus WT (R) mice at 48 days (D48) and then compared following perfusion with PBS (D48 Perfused). FIG. 14 B-C shows region-of-interest (ROI) generated plots from the data at day 28. Concentration of radioactivity (μCi/mm³) is measured between blood pool and tissue.

FIG. 15 (A-G): Overview of SPECT imaging data in Tg2576 mouse brain; following administration of (A) 1 mg/kg [¹²⁵I]Agly-chBIIB037 (22 month Tg2576), (C) 1 mg/kg [¹²⁵I]huBIIB037 F(ab′)2 (22 month Tg2576), or (D) 1 mg/kg [¹²⁵I]huBIIB037 F(ab′)2 (3 month WT). (B) Quantitative brain atlas analysis of in vivo [¹²⁵I]Agly-chBIIB037 binding in aged Tg2576 mice (22 month). (E) Quantitative region of interest analysis of in vivo [¹²⁵I]huBIIB037F(ab′)2 binding in aged Tg2576 and WT mouse brain. (F) Comparison of select sagittal and coronal images arbitrarily scaled to show in vivo uptake and retention of [¹²⁵I]huBIIB037F(ab′)2 in Tg mice images. (G) Overall summary of quantitative ROI analysis of in vivo [¹²⁵I]huBIIB037 F(ab′)2 binding in aged Tg2576 and wild type mouse brain. Data represented as % ID/g in cortex normalized to that in cerebellum over four imaging time points.

FIG. 16: Representative day 48 images showing in vivo accumulation of [¹²⁵I]Agly-chBIIB037 (1 and 10 mpk) into CNS of Tg2576 aged mouse brain. CNS-specific in vivo accumulation was also tested in young Tg2576 mouse brain as well as using [¹²⁵I]P1.17 labeled isotype control (10 mpk).

FIG. 17: 5F3 immunostaining for Ab and SPECT images (left) at 24 h acquisition representing low and high uptake animals. Plot on right shows correlation of quantitative SPECT ROI in cortex versus ex-vivo amyloid area (2 sections per animal) for all animals imaged.

FIG. 18: Sampling of mouse brain for histological and autoradiographical analysis.

FIG. 19: Microautoradiography (mARG) in Tg mouse 101 and WT mouse 201 in the area of the hippocampus.

FIG. 20: mArg in Tg mouse 101 and immunohistochemistry (IHC) staining for Aβ in adjacent section in the area of the hippocampus.

FIG. 21: mARG in Tg mouse 101 and Thioflavin-S staining for Aβ in adjacent section in the area of the hippocampus.

FIG. 22: Biodistribution of [¹²⁵I]chBIIB037 in target and non-target tissues/organs in Tg and WT mice. Data expressed as % ID/g across tissues.

FIG. 23: Whole body SPECT images of in vivo [¹²⁵I]huBIIB037F(ab′)2 in Tg2576 and WT mice.

FIG. 24: Quantitative whole body analysis of in vivo biodistribution of [¹²⁵I]huBIIB037F(ab′)2 in non-target tissues of aged Tg2576 and young WT mice. Data are expressed as % ID/g across tissues from Tg2576 (solid bars) and WT (striped bars) mice. [H, heart; K, kidneys; L, liver; Lu, lungs; M, muscle; S, stomach; T, thyroid; WB, whole body].

### DETAILED DESCRIPTION

### I. Definitions

It is to be noted that the term “a” or “an” entity refers to one or more of that entity; for example, “an anti-Aβ antibody,” is understood to represent one or more antibodies which specifically bind to Aβ. As such, the terms “a” (or “an”), “one or more,” and “at least one” can be used interchangeably herein.

As used herein, the term “neurodegenerative disease” includes but is not limited to Alzheimer's Disease, mild cognitive impairment, fronto-temporal dementia, Lewy-body disease, Parkinson's disease, Pick's disease, Binswanger's disease; congophilic amyloid angiopathy, cerebral amyloid angiopathy, Down's syndrome, multi-infarct dementia, Huntington's Disease, Creutzfeldt-Jakob Disease, AIDS dementia complex, depression, anxiety disorder, phobia, Bell's Palsy, epilepsy, encephalitis, multiple sclerosis: neuromuscular disorders, neurooncological disorders, brain tumors, neurovascular disorders including stroke, neuroimmunological disorders, neurootological disease, neurotrauma including spinal cord injury, pain including neuropathic pain, pediatric neurological and neuropsychiatric disorders, sleep disorders, Tourette syndrome, mild cognitive impairment, vascular dementia, multi-infarct dementia, cystic fibrosis, Gaucher's disease other movement disorders and disease of the central nervous system (CNS) in general. Unless stated otherwise, the terms “neurodegenerative,” “neurological” or “neuropsychiatric” are used interchangeably herein.

Unless stated otherwise, the terms “disorder,” “disease” and “illness” are used interchangeably herein.

Unless stated otherwise, the terms “Aβ,” “Abeta” and “beta-amyloid” are used interchangeably herein.

As used herein, the terms “binding molecule” or “antigen binding molecule” refers in its broadest sense to a molecule that specifically binds an antigenic determinant. Non-limiting examples of antigen binding molecules are antibodies and fragments thereof that retain antigen-specific binding, as well as other non-antibody molecules that bind to Aβ including but not limited to hormones, receptors, ligands, major histocompatibility complex (MHC) molecules, chaperones such as heat shock proteins (HSPs) as well as cell-cell adhesion molecules such as members of the cadherin, intergrin, C-type lectin and immunoglobulin (Ig) superfamilies. Thus, for the sake of clarity only and without restricting the scope of the disclosure most of the following embodiments are discussed with respect to antibodies and antibody-like molecules which represent the binding molecules for the development of therapeutic and diagnostic agents. In another embodiment, a binding molecule disclosed comprises at least one heavy or light chain CDR of an antibody molecule. In another embodiment, a binding molecule disclosed comprises at least two CDRs from one or more antibody molecules. In another embodiment, a binding molecule disclosed comprises at least three CDRs from one or more antibody molecules. In another embodiment, a binding molecule as disclosed comprises at least four CDRs from one or more antibody molecules. In another embodiment, a binding molecule as disclosed comprises at least five CDRs from one or more antibody molecules. In another embodiment, a binding molecule as disclosed comprises at least six CDRs from one or more antibody molecules.

Disclosed herein a method of reducing brain amyloid plaques without affecting vascular amyloid, comprising administering to a subject an anti-Aβ antibody or antigen-binding fragment thereof. Unless specifically referring to full-sized antibodies such as naturally occurring antibodies, the term “anti-Aβ antibody” encompasses full-sized antibodies as well as antigen-binding fragments, variants, analogs, or derivatives of such antibodies, e.g., naturally occurring antibody or immunoglobulin molecules or engineered antibody molecules or fragments that bind antigen in a manner similar to antibody molecules.

The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al. (1988) Antibodies: A Laboratory Manual (2nd ed.; Cold Spring Harbor Laboratory Press).

As used herein, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the disclosure and, accordingly, are within the scope of the disclosure. All immunoglobulin classes are clearly within the scope of the disclosure. The following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL or VK) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains actually comprise the carboxy-terminus of the heavy and light chain, respectively.

As indicated above, the variable region allows the antibody to selectively recognize and specifically bind epitopes on antigens. That is, the VL domain and VH domain, or subset of the complementarity determining regions (CDRs) within these variable domains, of an antibody combine to form the variable region that defines a three dimensional antigen binding site. This quaternary antibody structure forms the antigen binding site present at the end of each arm of the Y. More specifically, the antigen binding site is defined by three CDRs on each of the VH and VL chains. In some instances, e.g., certain immunoglobulin molecules derived from camelid species or engineered based on camelid immunoglobulins, a complete immunoglobulin molecule can consist of heavy chains only, with no light chains. See, e.g., Hamers-Casterman et al., Nature 363:446-448 (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops that connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable domain by one of ordinary skill in the art, since they have been precisely defined (see below).

In the case where there are two or more definitions of a term that is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al. (1983) U.S. Dept. of Health and Human Services, “Sequences of Proteins of Immunological Interest” and by Chothia and Lesk, J. Mol. Biol. 196:901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues that encompass the CDRs as defined by each of the above cited references are set forth below in Table 1 as a comparison. The exact residue numbers that encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

¹Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al. (1983) U.S. Dept. of Health and Human Services, “Sequence of Proteins of Immunological Interest.” Unless otherwise specified, references to the numbering of specific amino acid residue positions in an anti-Aβ antibody or antigen-binding fragment, variant, or derivative thereof of the present disclosure are according to the Kabat numbering system.

Antibodies or antigen-binding fragments, variants, or derivatives thereof of the disclosure include, but are not limited to, polyclonal, monoclonal, multispecific, human, humanized, primatized, or chimeric antibodies, single-chain antibodies, epitope-binding fragments, e.g., Fab, Fab′ and F(ab′)₂, Fd, Fvs, single-chain Fvs (scFv), disulfide-linked Fvs (sdFv), fragments comprising either a VL or VH domain, fragments produced by a Fab expression library, and anti-idiotypic (anti-Id) antibodies. ScFv molecules are known in the art and are described, e.g., in U.S. Pat. No. 5,892,019. Immunoglobulin or antibody molecules of the disclosure can be of any type (e.g., IgG, IgE, IgM, IgD, IgA, and IgY), class (e.g., IgG1, IgG2, IgG3, IgG4, IgA1, and IgA2, etc.), or subclass of immunoglobulin molecule.

As used herein, the term “heavy chain portion” includes amino acid sequences derived from an immunoglobulin heavy chain. In certain embodiments, a polypeptide comprising a heavy chain portion comprises at least one of: a VH domain, a CH1 domain, a hinge (e.g., upper, middle, and/or lower hinge region) domain, a CH2 domain, a CH3 domain, or a variant or fragment thereof. For example, a binding polypeptide for use in the disclosure can comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the disclosure comprises a polypeptide chain comprising a CH3 domain. Further, a binding polypeptide for use in the disclosure can lack at least a portion of a CH2 domain (e.g., all or part of a CH2 domain). As set forth above, it will be understood by one of ordinary skill in the art that these domains (e.g., the heavy chain portions) can be modified such that they vary in amino acid sequence from the naturally occurring immunoglobulin molecule.

In certain embodiments, anti-Aβ antibodies, or antigen-binding fragments, variants, or derivatives thereof disclosed herein, the heavy chain portions of one polypeptide chain of a multimer are identical to those on a second polypeptide chain of the multimer. Alternatively, heavy chain portion-containing monomers of the disclosure are not identical. For example, each monomer can comprise a different target binding site, forming, for example, a bispecific antibody.

The heavy chain portions of a binding molecule for use in the methods disclosed herein can be derived from different immunoglobulin molecules. For example, a heavy chain portion of a polypeptide can comprise a C_{H1} domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain, e.g., a kappa or lambda light chain. In certain embodiments, the light chain portion comprises at least one of a VL or CL domain.

As previously indicated, the subunit structures and three-dimensional configuration of the constant regions of the various immunoglobulin classes are well known. As used herein, the term “V H domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat E A et al. op. cit. The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen-binding regions to move independently. Hinge regions can be subdivided into three distinct domains: upper, middle, and lower hinge domains (Roux et al., J. Immunol. 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by a disulfide bond and the two heavy chains are linked by two disulfide bonds at positions corresponding to 239 and 242 using the Kabat numbering system (position 226 or 229, EU numbering system).

Anti-Aβ antibodies, or antigen-binding fragments, variants, or derivatives thereof disclosed herein can be described or specified in terms of the epitope(s) or portion(s) of an antigen, e.g., a target polypeptide disclosed herein (e.g., Aβ) that they recognize or specifically bind. The portion of a target polypeptide that specifically interacts with the antigen binding domain of an antibody is an “epitope,” or an “antigenic determinant.” A target polypeptide can comprise a single epitope, but typically comprises at least two epitopes, and can include any number of epitopes, depending on the size, conformation, and type of antigen. Furthermore, it should be noted that an “epitope” on a target polypeptide can be or can include non-polypeptide elements, e.g., an epitope can include a carbohydrate side chain.

The minimum size of a peptide or polypeptide epitope for an antibody is thought to be about foul to five amino acids. Peptide or polypeptide epitopes can contain, e.g., at least seven, at least nine or between at least about 15 to about 30 amino acids. Since a CDR can recognize an antigenic peptide or polypeptide in its tertiary form, the amino acids comprising an epitope need not be contiguous, and in some cases, can not even be on the same peptide chain. A peptide or polypeptide epitope recognized by anti-Aβ antibodies of the disclosure can contain a sequence of at least 4, at least 5, at least 6, at least 7, at least 8, at least 9, at least 10, at least 15, at least 20, at least 25, or between about 15 to about 30 contiguous or non-contiguous amino acids of Aβ.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” can be deemed to have a higher specificity for a given epitope than antibody “B,” or antibody “A” can be said to bind to epitope “C” with a higher specificity than it has for related epitope “D.”

By “preferentially binds,” it is meant that the antibody specifically binds to an epitope more readily than it would bind to a related, similar, homologous, or analogous epitope. Thus, an antibody that “preferentially binds” to a given epitope would more likely bind to that epitope than to a related epitope, even though such an antibody can cross-react with the related epitope.

By way of non-limiting example, an antibody can be considered to bind a first epitope preferentially if it binds said first epitope with a dissociation constant (K_{D}) that is less than the antibody's K_{D} for the second epitope. In another non-limiting example, an antibody can be considered to bind a first antigen preferentially if it binds the first epitope with an affinity that is at least one order of magnitude less than the antibody's K_{D} for the second epitope. In another non-limiting example, an antibody can be considered to bind a first epitope preferentially if it binds the first epitope with an affinity that is at least two orders of magnitude less than the antibody's K_{D} for the second epitope.

In another non-limiting example, an antibody can be considered to bind a first epitope preferentially if it binds the first epitope with an off rate (k(off)) that is less than the antibody's k(off) for the second epitope. In another non-limiting example, an antibody can be considered to bind a first epitope preferentially if it binds the first epitope with an affinity that is at least one order of magnitude less than the antibody's k(off) for the second epitope. In another non-limiting example, an antibody can be considered to bind a first epitope preferentially if it binds the first epitope with an affinity that is at least two orders of magnitude less than the antibody's k(off) for the second epitope.

An antibody or antigen-binding fragment, variant, or derivative disclosed herein can be said to bind a target polypeptide disclosed herein (e.g., Aβ) or a fragment or variant thereof with an off rate (k(off)) of less than or equal to 5×10⁻² sec⁻¹, 10⁻² sec⁻¹, 5×10⁻³ sec⁻¹ or 10⁻³ sec⁻¹. In certain embodiments, an antibody of the disclosure can be said to bind a target polypeptide disclosed herein (e.g., human AO) or a fragment or variant thereof with an off rate (k(off)) less than or equal to 5×10⁴ sec⁻¹, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹ or 10⁻⁷ sec⁻¹.

An antibody or antigen-binding fragment, variant, or derivative disclosed herein can be said to bind a target polypeptide disclosed herein (e.g., AO) or a fragment or variant thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. In certain embodiments, an antibody of the disclosure can be said to bind a target polypeptide disclosed herein (e.g., Aβ) or a fragment or variant thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

An antibody is said to competitively inhibit binding of a reference antibody to a given epitope if it preferentially binds to that epitope to the extent that it blocks, to some degree, binding of the reference antibody to the epitope. Competitive inhibition can be determined by any method known in the art, for example, competition ELISA assays. An antibody can be said to competitively inhibit binding of the reference antibody to a given epitope by at least 90%, at least 80%, at least 70%, at least 60%, or at least 50%. As described herein, an anti-A/3 antibody or antigen-binding fragment thereof competitively inhibits BIIB037 antibody.

As used herein, the term “affinity” refers to a measure of the strength of the binding of an individual epitope with the CDR of an immunoglobulin molecule. See, e.g., Harlow et al. (1988) Antibodies: A Laboratory Manual (Cold Spring Harbor Laboratory Press, 2nd ed.) pages 27-28.

Anti-Aβ antibodies or antigen-binding fragments, variants, or derivatives thereof as disclosed herein bind to parenchymal amyloid plaques with a greater affinity than to vascular amyloid.

As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity.

Anti-Aβ antibodies or antigen-binding fragments, variants, or derivatives thereof as disclosed herein can also be described or specified in terms of their cross-reactivity. As used herein, the term “cross-reactivity” refers to the ability of an antibody, specific for one antigen, to react with a second antigen; a measure of relatedness between two different antigenic substances. Thus, an antibody is cross-reactive if it binds to an epitope other than the one that induced its formation. The cross-reactive epitope generally contains many of the same complementary structural features as the inducing epitope, and in some cases, can actually fit better than the original.

For example, certain antibodies have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody can be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody can be deemed “highly specific” for a certain epitope, if it does not bind any other analog, ortholog, or homolog of that epitope.

Anti-Aβ antibodies or antigen-binding fragments, variants or derivatives thereof, as described herein can also be described or specified in terms of their binding affinity to a polypeptide of the disclosure, e.g., Aβ. Binding affinities can include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Antibody fragments including single-chain antibodies can comprise the variable region(s) alone or in combination with the entirety or a portion of the following: hinge region, CH1, CH2, and CH3 domains. Also included are antigen-binding fragments comprising any combination of variable region(s) with a hinge region, CH1, CH2, and CH3 domains. Binding molecules, e.g., antibodies, or antigen-binding fragments thereof disclosed herein can be from any animal origin including birds and mammals. The antibodies can be human, murine, donkey, rabbit, goat, guinea pig, camel, llama, horse, or chicken antibodies. In another embodiment, the variable region can be condricthoid in origin (e.g., from sharks).

As used herein, the term “chimeric antibody” will be held to mean any antibody wherein the immunoreactive region or site is obtained or derived from a first species and the constant region (which can be intact, partial or modified in accordance with the instant disclosure) is obtained from a second species. For example, the target binding region or site can be from a non-human source (e.g., mouse or primate) and the constant region can be human. Alternatively, a fully human binding region can be combined with a non-human (e.g., mouse) constant region.

As used herein, the term “murinized antibody” or “murinized immunoglobulin” refers to an antibody comprising one or more CDRs from a human antibody of the present disclosure; and a human framework region that contains amino acid substitutions and/or deletions and/or insertions that are based on a mouse antibody sequence. The human immunoglobulin providing the CDRs is called the “parent” or “acceptor” and the mouse antibody providing the framework changes is called the “donor”, Constant regions need not be present, but if they are, they are usually substantially identical to mouse antibody constant regions, i.e. at least about 85-90% or about 95% or more identical. Hence, in some embodiments, a full length murinized human heavy or light chain immunoglobulin contains a mouse constant region, human CDRs, and a substantially human framework that has a number of “murinizing” amino acid substitutions, Typically, a “murinized antibody” is an antibody comprising a murinized variable light chain and/or a murinized variable heavy chain. For example, a murinized antibody would not encompass a typical chimeric antibody, e.g., because the entire variable region of a chimeric antibody is non-mouse. A modified antibody that has been “murinized” by the process of “murinization” binds to the same antigen as the parent antibody that provides the CDRs and is usually less immunogenic in mice, as compared to the parent antibody.

As used herein, the term “engineered antibody” refers to an antibody in which the variable domain in either the heavy or light chain or both is altered by at least partial replacement of one or more CDRs from an antibody of known specificity and, if necessary, by partial framework region replacement and sequence changing. Although the CDRs can be derived from an antibody of the same class or even subclass as the antibody from which the framework regions are derived, it is envisaged that the CDRs will be derived from an antibody of different class, e.g., from an antibody from a different species. An engineered antibody in which one or more “donor” CDRs from a non-human antibody of known specificity is grafted into a human heavy or light chain framework region is referred to herein as a “humanized antibody.” It may not be necessary to replace all of the CDRs with the complete CDRs from the donor variable domain to transfer the antigen binding capacity of one variable domain to another. Rather, it can only be necessary to transfer those residues that are necessary to maintain the activity of the target binding site.

As used herein, “human” or “fully human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example, in U.S. Pat. No. 5,939,598 by Kucherlapati et al. “Human” or “fully human” antibodies also include antibodies comprising at least the variable domain of a heavy chain, or at least the variable domains of a heavy chain and a light chain, where the variable domain(s) have the amino acid sequence of human immunoglobulin variable domain(s).

“Human” or “fully human” antibodies also include “human” or “fully human” antibodies, as described herein, that comprise, consist essentially of, or consist of, variants (including derivatives) of antibody molecules (e.g., the VH regions and/or VL regions) described herein, which antibodies or fragments thereof immunospecifically bind to an Aβ polypeptide or fragment or variant thereof. Standard techniques known to those of skill in the art can be used to introduce mutations in the nucleotide sequence encoding a human anti-Aβ antibody, including, but not limited to, site-directed mutagenesis and PCR-mediated mutagenesis which result in amino acid substitutions. In certain embodiments the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH region, VHCDR1, VHCDR2, VHCDR3, VL region, VLCDR1, VLCDR2, or VLCDR3.

In one aspect, the antibody of the disclosure is a human monoclonal antibody as derived from human B cells. Optionally, the framework region of the human antibody is aligned and adopted in accordance with the pertinent human germ line variable region sequences in the database; see, e.g., Vbase (http://vbase.mrc-cpe.cam.ac.uk/) hosted by the MRC Centre for Protein Engineering (Cambridge, UK). For example, amino acids considered to potentially deviate from the true germ line sequence could be due to the PCR primer sequences incorporated during the cloning process. Compared to artificially generated human-like antibodies such as single chain antibody fragments (scFvs) from a phage displayed antibody library or xenogeneic mice the human monoclonal antibody of the present disclosure is characterized by (i) being obtained using the human immune response rather than that of animal surrogates, i.e., the antibody has been generated in response to natural Aβ in its relevant conformation in the human body, (ii) having protected the individual or is at least significant for the presence of Aβ, and (iii) since the antibody is of human origin the risks of cross-reactivity against self-antigens is minimized. Thus, in accordance with the disclosure the terms “human monoclonal antibody,” “human monoclonal autoantibody,” “human antibody” and the like are used to denote an Aβ binding molecule which is of human origin, i.e., which has been isolated from a human cell such as a B cell or hybridoma thereof or the cDNA of which has been directly cloned from mRNA of a human cell, for example a human memory B cell. A human antibody is still “human” even if amino acid substitutions are made in the antibody, e.g., to improve binding characteristics.

Antibodies derived from human immunoglobulin libraries or from animals transgenic for one or more human immunoglobulins and that do not express endogenous immunoglobulins, as described infra and, for example in, U.S. Pat. No. 5,939,598 by Kucherlapati et al., are denoted human-like antibodies in order distinguish them from truly human antibodies of the present disclosure.

As used herein, the terms “treat” or “treatment” refer to both therapeutic treatment and prophylactic or preventative measures, wherein the object is to prevent or slow down (lessen) an undesired physiological change, infection, or disorder. Beneficial or desired clinical results include, but are not limited to, alleviation of symptoms, diminishment of extent of disease, stabilized (i.e., not worsening) state of disease, clearance or reduction of an infectious agent in a subject, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the infection, condition, or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, bears, and so on.

### II. Anti-Aβ Antibodies

As disclosed herein, an anti-Aβ antibody or antigen-binding fragment thereof that binds to the same epitope as BIIB037 antibody, wherein BIIB037 antibody binds to an epitope comprising amino acids 3-6 of Aβ. Human BIIB037 antibody is described as NI-101.12F6A, in the International Publication No. WO2008/081008 incorporated herein by reference in its entirety. Unless stated otherwise, the terms “12F6A,” “hu12F6A” and “BIIB037” are used interchangeably herein. The term “chBIIB037” as disclosed herein refers to a murine chimeric version of BIIB037.

The human and chimeric anti-Aβ antibodies or antigen-binding fragments thereof, as described herein, specifically bind to Aβ and epitopes thereof and to various confirmations: of Aβ and epitopes thereof. For example, disclosed herein are antibodies or antigen-binding fragments thereof that selectively bind to Aβ aggregates. As used herein, reference to an antibody that “selectively binds,” “specifically binds,” or “preferentially binds” Aβ refers to an antibody that does not bind other unrelated proteins. An antibody that “selectively binds” or “specifically binds” Aβ conformer refers to an antibody that does not bind all conformations of Aβ, i.e., does not bind at least one other Aβ conformer. For example, disclosed herein are antibodies or antigen-binding fragments thereof that can distinguish among monomeric and aggregated forms of AO, i.e., bind to Aβ fibril but not Aβ monomer.

In certain embodiments, an anti-Aβ antibody or antigen-binding fragment, variant, or derivative thereof useful in the methods provided herein has an amino acid sequence that has at least about 80%, about 85%, about 88%, about 89%, about 90%, about 91%, about 92%, about 93%, about 94%, or about 95% sequence identity to the amino acid sequence of BIIB037 antibody. In a further embodiment, the binding molecule shares at least about 96%, about 97%, about 98%, about 99%, or 100% sequence identity to BIIB037 antibody. In certain embodiments, an anti-Aβ antibody or antigen-binding fragment, variant, or derivative thereof specifically binds to the same Aβ epitope as BIIB037 antibody. In some embodiments, an anti-Aβ antibody or antigen-binding fragment, variant, or derivative thereof comprises an immunoglobulin heavy chain variable region (VH) and an immunoglobulin light chain variable region (VL), wherein the VH comprises amino acid sequence at least 80%, 85%, 90% 95% or 100% identical to SEQ ID NO: 1 and the VL comprises amino acid sequence at least 80%, 85%, 90% 95% or 100% identical to SEQ ID NO: 2, as shown in Table 2.

In some embodiments, an anti-Aβ antibody or antigen-binding fragment, variant, or derivative thereof comprises VH and a VL, wherein the VH comprises amino acid sequence identical to, or identical except for one, two, three, four, five, or more amino acid substitutions to SEQ ID NO: 1, and the VL comprises amino acid sequence identical to, or identical except for one, two, three, four, five, or more amino acid substitutions to SEQ ID NO: 2, as shown in Table 2.

Some embodiments include an anti-Aβ antibody or antigen-binding fragment, variant, or derivative thereof useful in the methods provided herein which comprises a VH, where one or more of the VHCDR1, VHCDR2 or VHCDR3 regions of the VH are at least 80%, 85%, 90%, 95% or 100% identical to one or more reference heavy chain VHCDR2 and/or VHCDR3 amino acid sequences of one or more of: SEQ ID NO: 3, SEQ ID NO: 4, SEQ ID NO: 5, as shown in Table 3.

Further disclosed is an anti-Aβ antibody or antigen-binding fragment, variant, or derivative thereof useful in the methods provided herein which comprises a VH, where one or more of the VHCDR1, VHCDR2 or VHCDR3 regions of the VH are identical to, or identical except for four, three, two, or one amino acid substitutions, to one or more reference heavy chain VHCDR1, VHCDR2 or VHCDR3 amino acid sequences of one or more of: SEQ ID NO: 3, SEQ ID NO: 4, SEQ ID NO: 5, as shown in Table 3.

Also disclosed is an anti-Aβ antibody or antigen-binding fragment, variant, or derivative thereof useful in the methods provided herein which comprises a VL, Where one or more of the VLCDR1, VLCDR2 or VLCDR3 regions of the VL are at least 80%, 85%, 90%, 95% or 100% identical to one or more reference heavy chain VLCDR1, VLCDR2 or VLCDR3 amino acid sequences of one or more of: SEQ ID NO: 6, SEQ ID NO: 7, SEQ ID NO: 8, as shown in Table 3.

In some embodiments, an anti-Aβ antibody or antigen-binding fragment, variant, or derivative thereof useful in the methods provided herein comprises a VL, where one or more of the VLCDR1, VLCDR2 or VLCDR3 regions of the VL are identical to, or identical except for four, three, two, or one amino acid substitutions, to one or more reference heavy chain VLCDR1, VLCDR2 or VLCDR3 amino acid sequences of one or more of: SEQ ID NO: 6, SEQ ID NO: 7, SEQ ID NO: 8, as shown in Table 3.

In other embodiments, an anti-Aβ antibody or antigen-binding, fragment, variant, or derivative thereof useful in the methods provided herein comprises, consists essentially of, or consists of VH and VL amino acid sequences at least 80%, 85%, 90% 95% or 100% identical to: SEQ ID NO: 1 and SEQ ID NO: 2, as shown in Table 2.

In some embodiments, an anti-Aβ antibody or antigen-binding fragment, variant, or derivative thereof useful in the methods provided herein comprises BIIB037 antibody.

TABLE 2
BIIB037 antibody VH and VL amino acid sequences

| VH | VL |
|---|---|
| QVQLVESGGGVVQPGRSLRLSCA | DIQMTQSPSSLSASVGDRVTITC |
| ASGFAFSSYGMHWVRQAPGKGLE | RASQSISSYLNWYQQKPGKAPKL |
| WVAVIWFDGTKKYYTDSVKGRFT | LIYAASSLQSGVPSRFSGSGSGT |
| ISRDNSKNTLYLQMNTLRAEDTA | DFTLTISSLQPEDFATYYCQQSY |
| VYYCARDRGIGARRGPYYMDVWG | STPLTFGGGTKVEIKR |
| KGTTVTVSS | SEQ ID NO: 2 |
| SEQ ID NO: 1 | |

TABLE 3
BIIB037 Antibody VH and VL CDR1, CDR2,
and CDR3 amino acid sequences

| VHCDR1 | VHCDR2 | VHCDR3 | VLCDR1 | VLCDR2 | VLCDR3 |
|---|---|---|---|---|---|
| SYGMH | VIWFDGTKK | DRGIGAR | RASQSI | AASSLQS | QQSYSTP |
| SEQ ID | YYTDSVKG | RGPYYMDV | SSYLN | SEQ ID | LT |
| NO: 3 | SEQ ID | SEQ ID | SEQ ID | NO: 7 | SEQ ID |
| | NO: 4 | NO: 5 | NO: 6 | | NO: 8 |

Also included for use in the methods described herein are polypeptides encoding anti-Aβ antibodies, or antigen-binding fragments, variants, or derivatives thereof as described herein, polynucleotides encoding such polypeptides, vectors comprising such polynucleotides, and host cells comprising such vectors or polynucleotides, all for producing anti-Aβ antibodies, or antigen-binding fragments, variants, or derivatives thereof for use in the methods described herein.

Suitable biologically active variants of anti-Aβ antibodies as described herein can be used in the methods of the disclosure. Such variants will retain the desired binding properties of the parent anti-Aβ antibody. Methods for making antibody variants are generally available in the art.

Methods for mutagenesis and nucleotide sequence alterations are well known in the art. See, for example, Walker and Gaastra, eds. (1983) Techniques in Molecular Biology (MacMillan Publishing Company, New York); Kunkel, Proc. Natl. Acad Sci. USA 82:488-492 (1985); Kunkel et al., Methods Enzymol. 154:367-382 (1987); Sambrook et al. (1989) Molecular Cloning: A Laboratory Manual (Cold Spring Harbor, N.Y.); U.S. Pat. No. 4,873,192; and the references cited therein; herein incorporated by reference. Guidance as to appropriate amino acid substitutions that do not affect biological activity of the polypeptide of interest can be found in the model of Dayhoff et al. in Atlas of Protein Sequence and Structure (Natl. Biomed. Res. Found., Washington, D.C.), pp. 345-352 (1978), herein incorporated by reference in its entirety. The model of Dayhoff et al. uses the Point Accepted Mutation (PAM) amino acid similarity matrix (PAM 250 matrix) to determine suitable conservative amino acid substitutions. Conservative substitutions, such as exchanging one amino acid with another having similar properties, can be made. Examples of conservative amino acid substitutions as taught by the PAM 250 matrix of the Dayhoff et al. model include, but are not limited to, Gly⇄Ala, Val⇄Ile⇄Leu, Asp⇄Glu, Lys⇄Arg, Asn⇄Gln, and Phe⇄Trp⇄Tyr.

Methods for measuring an anti-Aβ antibody or antigen-binding fragment, variant, or derivative thereof, binding specificity include, but are not limited to, standard competitive binding assays, assays for monitoring immunoglobulin secretion by T cells or B cells, T cell proliferation assays, apoptosis assays, ELISA assays, and the like. See, for example, such assays disclosed in WO 93/14125; Shi et al., Immunity 13:633-642 (2000); Kumanogoh et al., J Immunol 169:1175-1181 (2002); Watanabe et al., J Immunol 167:4321-4328 (2001); Wang et al., Blood 97:3498-3504 (2001); and Giraudon et al., J Immunol 172(2):1246-1255 (2004), all of which are herein incorporated by reference. The term “percent sequence identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity.

When discussed herein whether any particular polypeptide, including the constant regions, CDRs, VH domain or VL domains disclosed herein, is at least about 65%, about 70%, about 75%, about 80%, about 85%, about 90%, about 91%, about 92%, about 93%, about 94%, about 95%, about 96%, about 97%, about 98%, about 99%, or even about 100% identical to another polypeptide, the comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is b12seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculated percent sequence identity can be curated either automatically or manually.

A variant can, for example, differ from a reference anti-Aβ antibody by as few as 1 to 15 amino acid residues, as few as 1 to 10 amino acid residues, such as 6-10, as few as 5, as few as 4, 3, 2, or even 1 amino acid residue. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind an Aβ polypeptide).

For example, it is possible to introduce mutations only in framework regions or only in CDR regions of an antibody molecule. Introduced mutations can be silent or neutral missense mutations, i.e., have no, or little, effect on an antibody's ability to bind antigen. These types of mutations can be useful to optimize codon usage, or improve a hybridoma's antibody production. Alternatively, non-neutral missense mutations can alter an antibody's ability to bind antigen. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein can routinely be expressed and the functional and/or biological activity of the encoded protein, (e.g., ability to immunospecifically bind at least one epitope of an Aβ polypeptide) can be determined using techniques described herein or by routinely modifying techniques known in the art.

### III. Treatment Methods Using Anti-Aβ Antibodies

The present disclosure relates to a method for reducing brain amyloid plaques or minimizes the occurrence of microhemorrhage during chronic dosing of an anti-Aβ or antigen-binding fragment thereof, comprising administering to a subject an anti-Aβ antibody or antigen-binding fragment thereof.

The term “minimizing” as used herein means reducing, preventing, keeping constant, not increasing, etc.

In certain embodiments, the methods as described herein are directed to reducing brain amyloid plaques, comprising administering to a subject the anti-Aβ antibody or antigen-binding fragment thereof that binds to the same epitope as BIIB037 antibody, wherein the administration can reduce amyloid plaques in brain without substantially affecting vascular amyloid, and wherein BIIB037 antibody binds to an epitope comprising amino acids 3-6 of Aβ.

In some embodiments, the methods as described herein are directed to reducing brain amyloid, comprising administering to a subject an anti-Aβ antibody or antigen-binding fragment thereof that competitively inhibits BIIB037 antibody, wherein the administration can reduce amyloid plaques in brain without substantially affecting vascular amyloid.

The term “reducing brain amyloid plaques without substantially affecting vascular amyloid” as used herein means that the amount of vascular amyloid deposition is neither substantially reduced nor substantially increased by an anti-Aβ antibody or antigen-binding fragment thereof treatment compared to the control (i.e., untreated) group.

Further provided is a method for reducing brain amyloid plaques, wherein reduction of amyloid plaques in brain involves Fc receptor engagement. One or more Fc receptors are expressed in microglia, astrocytes, oligodendrocytes and neurons. Fc receptors are increasingly recognized for their involvement in neurological disorders. The anti-Aβ antibody/Aβ immune complexes can be cleared by Fc-dependent activation of microglia followed by phagocytosis of Aβ deposits (Wilcock et al., J Neurosci. 23(9):3745-3751 (2003)) or Fc-independent mechanisms (Das et al., J Neurosci. 23(24):8532-8538 (2005)).

In some embodiments the methods as described herein are directed to the use of anti-Aβ antibodies, including antigen-binding fragments, variants, and derivatives thereof, described herein. Further provided is a method of minimizing the occurrence of microhemorrhage during chronic dosing of the anti-Aβ antibody or antigen-binding fragment thereof comprising administering to a subject the anti-Aβ antibody that binds to the same epitope as BIIB037 antibody, and wherein BIIB037 antibody binds to an epitope comprising amino acids 3-6 of Aβ. In certain embodiments the methods as described herein minimize the occurrence of microhemorrhage during chronic dosing of the anti-Aβ antibody or antigen-binding fragment thereof, comprising administering to a subject the anti-Aβ antibody that competitively inhibits BIIB037 antibody. By a further embodiment, anti-Aβ antibodies, including antigen-binding fragments, variants, and derivatives thereof, as described herein, can also be used in a method for the diagnosis or screening of a neurodegenerative disorder in an individual by obtaining a body fluid sample from the tested individual which can be a blood sample, a lymph sample, or any other body fluid sample, and contacting the body fluid sample with an anti-Aβ antibody as described herein, under conditions enabling the formation of antibody-antigen complexes. The level of such complexes is then determined by methods known in the art e.g., ELISA, immunofluorescence, fluorescence-activated cell sorter (FACS), magnetic cell sorter, a level significantly higher than that formed in a control sample indicating the disease in the tested individual. In the same manner, the specific antigen bound by the anti-Aβ antibodies as described herein can also be used. Thus, the disclosure relates to an in vitro immunoassay comprising an anti-Aβ binding molecule, e.g., antibody or antigen-binding fragment thereof of the disclosure.

By a further embodiment, anti-Aβ antibodies, including antigen-binding fragments, variants, and derivatives thereof, as described herein, can also be used in a method for treating or preventing the progression of a neurodegenerative disorder in an individual or for the amelioration of symptoms associated with a neurodegenerative disorder.

As used herein, the terms “treat” or “treatment” refer to both therapeutic treatment and prophylactic or preventative measures, wherein the object is to prevent or slow down (lessen) an undesired physiological change, infection, or disorder. Beneficial or desired clinical results include, but are not limited to, alleviation of symptoms, diminishment of extent of disease, stabilized (i.e., not worsening) state of disease, clearance or reduction of an infectious agent in a subject, a delay or slowing of disease progression, amelioration or palliation of the disease state, and remission (Whether partial or total), whether detectable or undetectable. “Treatment” can also mean prolonging survival as compared to expected survival if not receiving treatment. Those in need of treatment include those already with the infection, condition, or disorder as well as those prone to have the condition or disorder or those in which the condition or disorder is to be prevented. The term “method of treatment” as used herein covers the use of anti-Aβ antibodies, including antigen-binding fragments, variants, and derivatives thereof, described herein for treating or preventing the progression of a neurodegenerative disorder in an individual or for the amelioration of symptoms associated with a neurodegenerative disorder. The term “method of treatment” as used herein also covers the anti-Aβ antibodies, including antigen-binding fragments, variants, and derivatives thereof, described herein to treat or prevent the progression of a neurodegenerative disorder in an individual or to ameliorate symptoms associated with a neurodegenerative disorder.

In specific embodiments the methods as described herein are directed to treating or preventing the progression of Alzheimer's disease; for the amelioration of symptoms associated with Alzheimer's disease; for diagnosing or screening a subject for the presence of Alzheimer's disease or for determining a subject's risk for developing Alzheimer's disease.

The level of Aβ can be assessed by any suitable method known in the art comprising, e.g., analyzing Aβ by one or more techniques chosen from Western blot, immunoprecipitation, enzyme-linked immunosorbent assay (ELISA), radioimmunoassay (RIA), fluorescent activated cell sorting (FACS), two-dimensional gel electrophoresis, mass spectroscopy (MS), matrix-assisted laser desorption/ionization-time of flight-MS (MALDI-TOF), surface-enhanced laser desorption ionization-time of flight (SEMI-TOE), high performance liquid Chromatography (HPLC), fast protein liquid chromatography (FPLC), multidimensional liquid chromatography (LC) followed by tandem mass spectrometry (MS/MS), and laser densitometry. In certain embodiments, in vivo imaging of Aβ comprises positron emission tomography (PET), single photon emission tomography (SPECT), near infrared (NIR) optical imaging or magnetic resonance imaging (MRI).

As is well known in the medical arts, dosages for any one patient depends upon many factors, including the patients size, body surface area, age, the particular compound to be administered, sex, time and route of administration, general health, and other drugs being administered concurrently.

In certain embodiments, an antibody-based array can be used, which is for example loaded with anti-Aβ antibodies or equivalent antigen-binding molecules of the disclosure which specifically recognize Aβ. Design of microarray immunoassays is summarized in Kusnezow et al., Mol. Cell Proteomics 5:1681-1696 (2006). Accordingly, the disclosure also relates to microarrays loaded with anti-Aβ binding molecules identified in accordance with the present disclosure.

### IV. Diagnosing or Tracking Methods Using Anti-Aβ Antibodies

The present disclosure provides the use of anti-Aβ antibodies or antigen-binding fragments, variants, or derivatives thereof, for measuring the amount of brain amyloid plaques in a test subject, assessing disease progression in a patient being treated for a neurodegenerative disease or treating a neurodegenerative disease characterized by brain amyloid plaques in a patient in need of treatment.

In some embodiments, an anti-Aβ antibody or antigen-binding fragment, variant, or derivative thereof useful in the methods provided herein can be conjugated to therapeutic agents, prodrugs, peptides, proteins, enzymes, viruses, lipids, biological response modifiers, pharmaceutical agents, or PEG.

Conjugates that are immunotoxins including conventional antibodies have been widely described in the art. The toxins can be coupled to the antibodies by conventional coupling techniques or immunotoxins containing protein toxin portions can be produced as fusion proteins.

Examples of therapeutic agents which can be coupled to the anti-Aβ antibodies or antigen-binding fragments, variants, or derivatives thereof as disclosed herein for immunotherapy are drugs, radioisotopes, lectins, and toxins.

In using radioisotopically conjugated anti-Aβ antibodies or antigen-binding fragments, variants, or derivatives thereof useful in the methods provided herein for, e.g., immunotherapy, certain isotopes can be chosen depending on such factors as leukocyte distribution as well as stability and emission. Depending on the autoimmune response, some emitters can be used. In general, α and β particle emitting radioisotopes are utilized in immunotherapy. In certain embodiments, short range, high energy a emitters such as ²¹²Bi can be used. Examples of radioisotopes which can be bound to the anti-Aβ antibodies or antigen-binding fragments, variants, or derivatives thereof of the disclosure for therapeutic purposes include, but are not limited to ¹²³I, ¹²⁴I, ¹²⁵I, ¹³¹I, ⁸⁹Zr, ⁹⁰Y, ⁶⁷Cu, ⁶⁴Cu, ¹¹¹In, ²¹²Bi, ²¹²At, ²¹¹Pb, ⁴⁷Sc, ¹⁰⁹Pd, and ¹⁸⁸Re. Other therapeutic agents which can be coupled to the binding molecule, e.g., anti-Aβ antibodies or antigen-binding fragments, variants, or derivatives thereof disclosed herein, as well as ex vivo and in vivo therapeutic protocols, are known, or can be easily ascertained, by those of ordinary skill in the art. Wherever appropriate the person skilled in the art can use a polynucleotide of the invention encoding any one of the above-described antibodies, antigens or the corresponding vectors instead of the proteinaceous material itself.

Those skilled in the art will appreciate that conjugates can also be assembled using a variety of techniques depending on the selected agent to be conjugated. For example, conjugates with biotin are prepared e.g. by reacting a binding polypeptide with an activated ester of biotin such as the biotin N-hydroxysuccinimide ester. Similarly, conjugates with a fluorescent marker can be prepared in the presence of a coupling agent, e.g., by reaction with an isothiocyanate, e.g., fluorescein-isothiocyanate. Conjugates of the anti-Aβ antibodies or antigen-binding fragments, variants, or derivatives thereof of the disclosure are prepared in an analogous manner.

Further provided is a method of measuring the amount of brain amyloid plaques in a test subject, comprising: (a) measuring the signal generated in the brain of a test subject following administration of an anti-Aβ antibody or antigen-binding fragment thereof that binds to the same conformational epitope as the BIIB037 antibody or competitively inhibits BIIB037 antibody, wherein the antibody or antigen-binding fragment thereof is labeled with an agent that generates a measurable signal as described herein; and (b) comparing the signal generated in the test subject to a signal generated upon administration of the labeled antibody or antigen-binding fragment thereof to one or more control subjects; wherein an increase in the signal generated in the test subject relative to the control subject correlates with an increase in brain amyloid plaques.

The signal generated by the agent can be measured, for example, by single-photon emission computed tomography (SPECT) or positron emission tomography (PET).

Disclosed herein are the anti-Aβ antibodies or antigen-binding fragments, variants, or derivatives thereof conjugated to a diagnostic or therapeutic agent. Detection can be facilitated by coupling the anti-Aβ antibodies or antigen-binding fragments, variants, or derivatives thereof to a detectable substance, i.e., an agent that generates a measurable signal. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, e.g., U.S. Pat. No. 4,741,900 for metal ions which can be conjugated to antibodies for use as diagnostics according to the disclosure. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

By “control subject(s),” is meant any normal healthy subject (or a pool of subjects), a subject or subjects with different degrees of disease, or even the actual test subject at an earlier stage of disease.

Further provided is a method of assessing disease progression in a patient being treated for a neurodegenerative disease characterized by brain amyloid plaques, comprising: (a) administering an anti-Aβ antibody or antigen-binding fragment thereof that binds to the same conformational epitope as the BIIB037 antibody or that competitively inhibits the BIIB037 antibody to the patient in need of neurodegenerative disease treatment wherein the antibody or antigen-binding fragment thereof is labeled with an agent that generates a measurable signal as described herein, wherein the signal is measured in the patient following the administration; (b) administering the labeled anti-AP antibody or antigen-binding fragment thereof at one or more time intervals following the administration of (a), wherein the signal is measured in the patient following the administration (c) assessing disease progression in the patient based on a change in the signal measured in the patient at the one or more time intervals following administration of (a); wherein an increase in the signal indicates progression of the neurodegenerative disease in the patient.

The labeled anti-Aβ antibody or antigen-binding fragment thereof disclosed herein can be used diagnostically to, for example, monitor the development or progression of a neurodegenerative disease as part of a clinical testing procedure to, e.g., determine the efficacy of a given treatment and/or prevention regimen. The patient's treatment can be adjusted based on the level of neurodegenerative disease progression.

Further provided is a method of treating a neurodegenerative disease characterized by brain amyloid plaques in a patient in need of treatment, comprising: (a) administering an anti-Aβ antibody or antigen-binding fragment thereof that binds to the same conformational epitope as the BIIB037 antibody to a patient in need of neurodegenerative disease treatment, wherein the antibody or antigen-binding fragment thereof is labeled with an agent that generates a measurable signal;(b) assessing the disease state in the patient upon review of a comparison of the signal measured in the patient to the signal measured following administration of the labeled antibody or antigen-binding fragment thereof to one or more control subjects; wherein an increase in the signal generated in the patient relative to the control subject correlates with an increase in brain amyloid plaques; and (c) treating the patient with a therapy appropriate for the patient's disease state.

A therapy appropriate for the patient's state comprises administering an anti-AP antibody or antigen-binding fragment thereof that binds to the same conformational epitope as the BIIB037 antibody or that competitively inhibits the BIIB037 antibody to the patient in need of the neurodegenerative disease treatment. The a method of treating a neurodegenerative disease characterized by brain amyloid plaques as disclosed herein can further comprise (d) administering the labeled anti-Aβ antibody or antigen-binding fragment thereof at one or more time intervals following the administration of (a), wherein the signal is measured in the patient following the administration; and (e) assessing disease progression in the patient based on a change in the signal measured in the patient at the one or more time intervals following administration of (a); wherein an increase in the signal indicates progression of the neurodegenerative disease in the patient.

### V. Compositions and Administration Methods

The methods of preparing and administering anti-Aβ antibodies, or antigen-binding fragments, variants, or derivatives thereof to a subject in need thereof are well known to or are readily determined by those skilled in the art. The route of administration of an anti-Aβ antibody, or antigen-binding fragment, variant, or derivative thereof, can be, for example, peripheral, oral, parenteral, by inhalation or topical.

As discussed herein, anti-Aβ antibodies, or antigen-binding fragments, variants, or derivatives thereof can be formulated so as to facilitate administration and promote stability of the active agent. In certain embodiments, pharmaceutical compositions in accordance with the present disclosure comprise a pharmaceutically acceptable, non-toxic, sterile carrier such as physiological saline, non-toxic buffers, preservatives and the like. For the purposes of the instant application, a pharmaceutically effective amount of an anti-Aβ antibody, or antigen-binding fragment, variant, or derivative thereof, shall be held to mean an amount sufficient to achieve effective binding to a target and to achieve a benefit, e.g., reduce brain amyloid plaques without affecting vascular amyloid, or minimizes the occurrence of microhemorrhage during chronic dosing of the anti-Aβ antibody or antigen-binding fragment thereof. In some embodiments, an anti-Aβ antibody or antigen-binding fragment, variant, or derivative thereof can cross the blood-brain barrier in an effective amount to reduce brain amyloid plaques.

The pharmaceutical compositions used in this disclosure comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Prevention of the action of microorganisms can be achieved by various antibacterial and antifungal agents, for example, parabens, chlorobutanol, phenol, ascorbic acid, thimerosal and the like. In many cases, isotonic agents can be included, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

Parenteral formulations can be a single bolus dose, an infusion or a loading bolus dose followed with a maintenance dose. These compositions can be administered at specific fixed or variable intervals, e.g., once a day, or on an “as needed” basis.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like. Furthermore, the pharmaceutical composition of the disclosure can comprise further agents such as dopamine or psychopharmacologic drugs, depending on the intended use of the pharmaceutical composition. Furthermore, the pharmaceutical composition can also be formulated as a vaccine, for example, if the pharmaceutical composition of the disclosure comprises an anti-Aβ antibody for passive immunization.

Certain pharmaceutical compositions, as disclosed herein, can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of an anti-Aβ antibody, or fragment, variant, or derivative thereof, to be combined with the carrier materials to produce a single dosage form will vary depending upon the host treated and the particular mode of administration. The composition can be administered as a single dose, multiple doses or over an established period of time in an infusion. Dosage regimens also can be adjusted to provide the optimum desired response (e.g., a therapeutic or prophylactic response).

The term “peripheral administration” as used herein includes, e.g., intravenous, intraarterial, intraperitoneal, intramuscular, subcutaneous, rectal, or vaginal administration. While all these forms of administration are clearly contemplated as being within the scope of the disclosure, an example of a form for administration would be a solution for injection, in particular for intravenous or intraarterial injection or drip. A suitable pharmaceutical composition for injection can comprise a buffer (e.g., acetate, phosphate or citrate buffer), a surfactant (e.g., polysorbate), optionally a stabilizer agent (e.g., human albumin), etc. Preparations for peripheral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include, e.g., water, alcoholic/aqueous solutions, emulsions or suspensions, including saline and buffered media. In the subject disclosure, pharmaceutically acceptable carriers include, but are not limited to, 0.01-0.1 M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as, for example, antimicrobials, antioxidants, chelating agents, and inert gases and the like.

### ***

The practice of the disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Roitt, I., Brostoff, J. and Male D., Immunology, 6^{th} ed. London: Mosby (2001); Abbas A., Abul, A. and Lichtman, A., Cellular and Molecular Immunology, Ed. 5, Elsevier Health Sciences Division (2005); and Harlow and Lane, Antibodies: A Laboratory Manual, Cold Spring Harbor Press (1988).

Having now described the disclosure in detail, the same will be more clearly understood by reference to the following examples, which are included herewith for purposes of illustration only and are not intended to be limiting of the disclosure. All patents and publications referred to herein are expressly incorporated by reference in their entireties.

### EXAMPLES

Detailed descriptions of conventional methods, such as those employed herein can be found in the cited literature. Unless indicated otherwise below, identification of AO-specific B cells and molecular cloning of anti-Aβ antibodies displaying specificity of interest as well as their recombinant expression and functional characterization has been or can be performed as described in the Examples and Supplementary Methods section of international applications PCT/EP2008/000053 published as WO2008/081008, and international applications PCT/EP2009/009186 published as WO2010/069603, the disclosure content of which is incorporated herein by reference in its entirety.

### Example 1

### Generalization and In Vitro Characterization of Human BIIB037 (“BIIB037”) and chimeric BIIB037 (“chBIIB037”)

This example describes BIIB037 and chBIIB037 antibody generation. In addition, the example describes the binding affinities and selectivities of BIIB037 and chBIIB037 antibody for Aβ, which were assessed using series of biochemical methods described herein.

Cohorts of healthy elderly subjects with excellent cognitive performance or remission from mild cognitive impairment (MCI) or beginning Alzheimer's disease (AD) were screened for Aβ-reactive memory B cells. Positive B-cell clones were subjected to cDNA cloning and recombinant expression to generate human monoclonal antibodies against Aβ as described previously in WO2008/081008. Candidate antibodies were selected by their capacity to recognize both aggregated Aβ in vitro and amyloid plaques ex vivo using a tissue plaque immunoreactivity (TAPIR) assay in both AD and APP transgenic mice brain sections (Hock et al., Neuron 38:547-554 (2003)). One such candidate, a human IgG1/kappa monoclonal antibody designated BIIB037, was selected for further characterization, based on its capacity to recognize both aggregated Aβ in vitro and ex vivo beta-amyloid plaques on tissue sections from either human AD brains or APP transgenic mice (Hock et al, Nature Medicine 8(11):1270-1275 (2002)). A chimeric version of BIIB037 (i.e., chBIIB037) was generated, in which the constant regions of the heavy and light chain were replaced with murine IgG2a and murine kappa chain sequences, respectively. This chimeric molecule (chBIIB037) was used for chronic dosing studies in APP transgenic mice. The mIgG2a backbone of chBIIB037 binds with high affinity to mouse Fcγ receptors, making this antibody competent to recruit immune effector cells such as microglia in the brain.

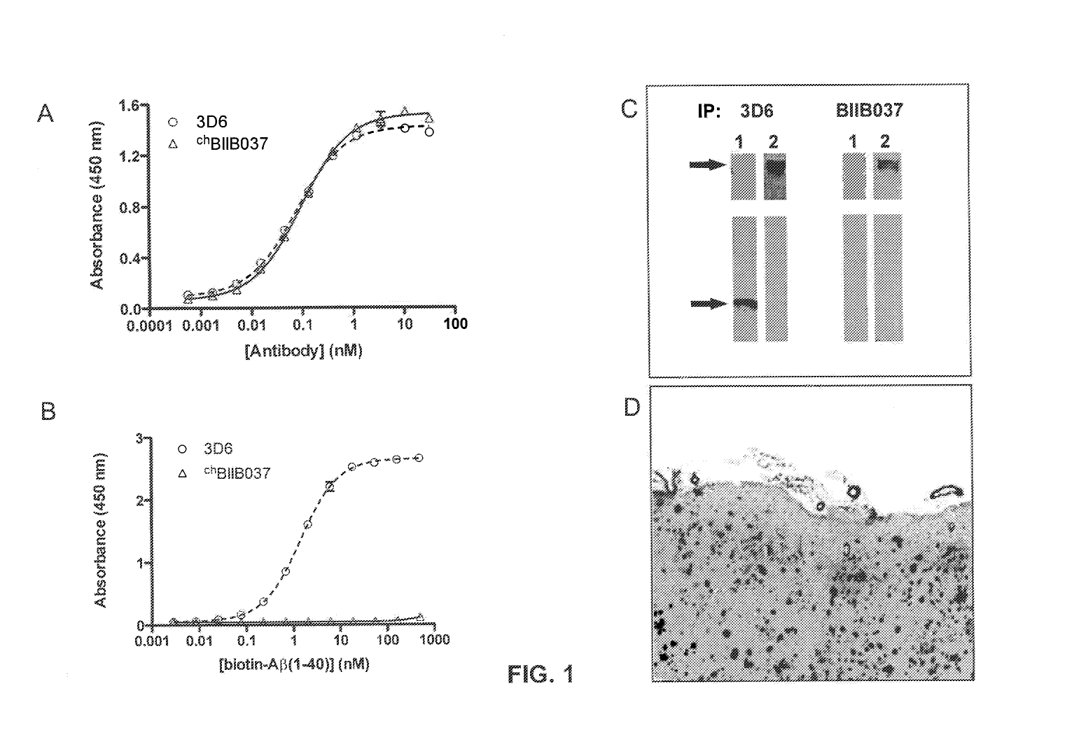

The binding affinities and selectivities of BIIB037 and chBIIB037 for Aβ were assessed using a series of biochemical methods. Aβ(1-42) aggregates were prepared by incubation at 37° C. and coated directly on an ELISA plate as described in WO2008/081008. When preformed fibrillar aggregates of Aβ were coated on an ELISA plate, chBIIB037 bound with an EC50 of 0.1 nM, comparable to the well-characterized antibody 3D6 (Bard et al., Nature Medicine 6:916-919 (2000)), (FIG. 1A). When chBIIB037 was immobilized and used to capture soluble, freshly prepared monomeric Aβ(1-40), no signal was observed, in contrast to 3D6, which captured soluble Aβ(1-40) with an EC50 of 1 nM (FIG. 1B). The inability of BIIB037 to bind soluble monomeric forms of Aβ was confirmed by immunoprecipitation studies using preparations of either freshly prepared monomeric Aβ1-42 or preformed fibrillar aggregates of Aβ (FIG. 1C). Specifically, preparations of aggregated or monomeric Aβ(1-42) were incubated with either BIIB037 or 3D6 and captured using protein A sepharose beads. The beads were washed, resuspended and boiled in SDS-PAGE sample buffer containing dithiothreitol, and the supernatants were loaded onto a 16% Tricine SDS-PAGE. Nitrocellulose blots were prepared and stained for total Aβ by Western blotting using the mouse anti-beta amyloid monoclonal antibody 6E10 (Covance, Princeton, N.J.), at the manufacturer's recommended dilution. Synthetic Aβ1-42 (Aβ42) peptide (Ana Spec, Fremont, CM was reconstituted in hexafluoroisopropanol at a concentration of 1 mg/L, air-dried to form a film, and dissolved in DMSO at a concentration of 1 mg/mL. Aβ42 amyloid fibrils were prepared by diluting DMSO-reconstituted monomeric Aβ42 into PBS at a concentration of 100 μg/mL, and incubating at 37° C. for at least 24 h. Samples of either freshly prepared monomeric or fibrillar Aβ42 were incubated with either BIIB037 or 3D6 and captured using protein A Sepharose beads. The beads were washed, resuspended and boiled in SDS-PAGE sample buffer containing dithiothreitol, the supernatants were loaded on a 16% Tricine SDS-PAGE gel and blotted to a nitrocellulose membrane, and Aβ42 was detected using the mouse monoclonal antibody 6E10 (Covance, Princeton, N.J.).

Consistent with the high binding avidity of the antibody for aggregates, chBIIB037 immunostained amyloid plaques in AD brain tissue (FIG. 1D). Tissues were formalin-fixed paraffin embedded, sectioned at 5 μm, deparaffinized and an EDTA-borate based heat-induced epitope retrieval (Ventana CC1) was used. Subsequently, FITC-labeled chBIIB037 was applied to tissues (1.7 μg/ml) with subsequent incubation with sheep-anti-FITC antibody at a 1:50 dilution. Tissues were then stained with hematoxylin.

### Example 2

### Brain Penetration of BIIB037 After a Single Intra-Peritoneal Administration in Tg2576 Transgenic Mice

This example describes the ability of BIIB037 to penetrate into the brain and bind to Aβ. Penetration of BIIB037 into the brain was assessed in 22 month old female Tg2576 mice (Kawarabayashi et al., J Neurosci 21(2):372-381 (2001)), following acute dosing, i.e., single dose of BIIB037 at 30 mg/kg administered intraperitoneally.

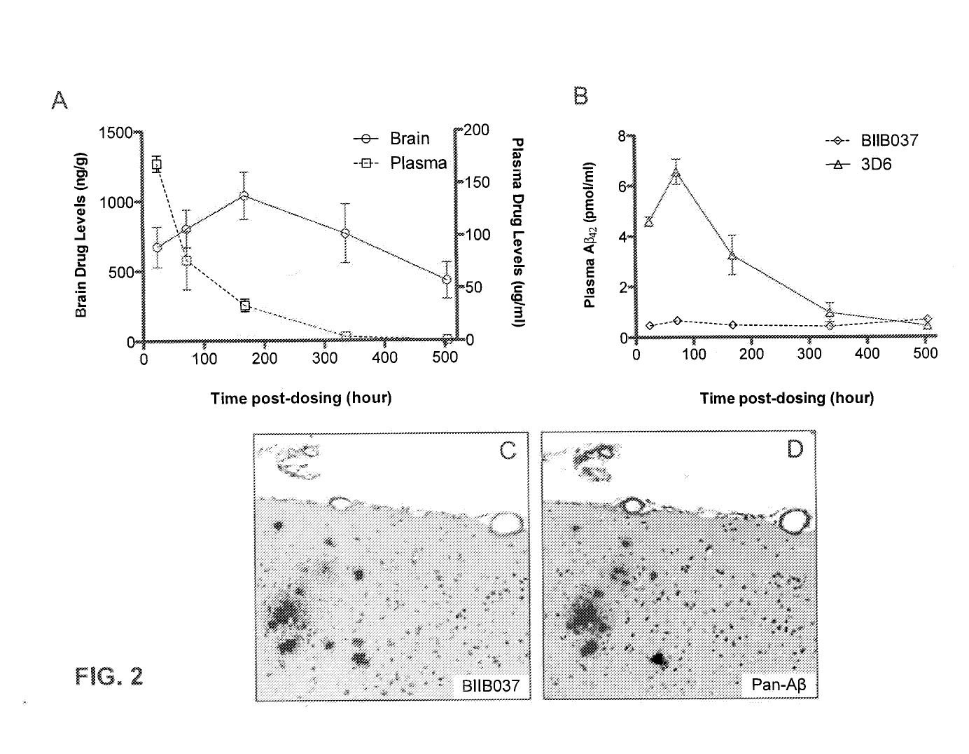

BIIB037 plasma and brain concentrations were determined by ELISA at 1 and 3 days, and 1, 2, and 3 weeks following administration of the single dose of the antibody (FIG. 2A). Specifically, frozen brains were homogenized in 10 volumes (10 mL/g of wet tissue) of a solution containing 50 mM NaCl, 0.2% diethylamine (DEA), with protease inhibitors, and sonicated for approximately 15-20 s on ice. The samples were then centrifuged at 100,000 g for 30 min at 4° C. The supernatant was retained as the DEA extracted soluble Aβ fraction. The remaining pellets were resuspended in 10 volumes of 5M guanidine-hydrochloride (Gu-HCl), sonicated, and centrifuged as above. The resultant supernatant was retained as the guanidine-extracted insoluble Aβ fraction, and the remaining pellet was discarded. For plasma and brain BIIB037 concentrations, 96 well microplates (Nunc Maxisorp, Corning Costar) were coated with Aβ(1-42) peptide (Aβ42) at a concentration of 5 ug/mL in cold coating buffer overnight at 4° C. Plasma or DEA extracted (i.e., detergent-free) brain homogenates samples were diluted to final working concentrations and incubated for 2 hours at room temperature. Binding was determined using a horseradish peroxidase (HRP)-conjugated goat anti-human polyclonal antibody (Jackson ImmunoResearch) followed by measurement of HRP activity using the substrate TME. Concentrations were determined by comparison to a standard curve generated using purified antibodies. Cmax in plasma was determined to be 181 μg/ml, with a half-life of 2.5 days. Cmax in brain was determined to be 1062 ng/g of tissue, and the half-life was 13 days. As shown in FIG. 2B plasma Aβ concentrations were unchanged after a single dose of BIIB037. The decline in brain BIIB037 levels did not parallel the decline in plasma, suggesting accumulation of the drug in the brain while it is cleared from plasma. In contrast, 3D6 antibody triggered a plasma Aβ spike.

In vivo binding of BIIB037 to Aβ deposits after a single dose in Tg2576 mice was revealed using a biotinylated anti-human secondary antibody, and was compared to the staining with the pan-Aβ antibody 5F3 performed ex vivo on a consecutive section. Systemically administered BIIB037 typically binds to parenchymal amyloid plaques with high affinity (FIG. 2C). Staining of parenchymal plaques by BIIB037 was observed 1 day post-injection, but was well-defined 3 days post-injection and was then similar to staining with the pan-Aβ antibody 5F3 (FIG. 2D). Tissues were formalin-fixed paraffin embedded, sectioned at 5 μm, deparaffinized and an EDTA-borate based heat-induced epitope retrieval (Ventana CC1) was used. Subsequently, a goat anti-human secondary antibody was applied to tissues at a 1:500 dilution. Tissues were then stained with hematoxylin.

### Example 3

### Binding of chBIIB037 to Parenchymal Amyloid Plaques Following Acute Dosing in Tg2576 Transgenic Mice

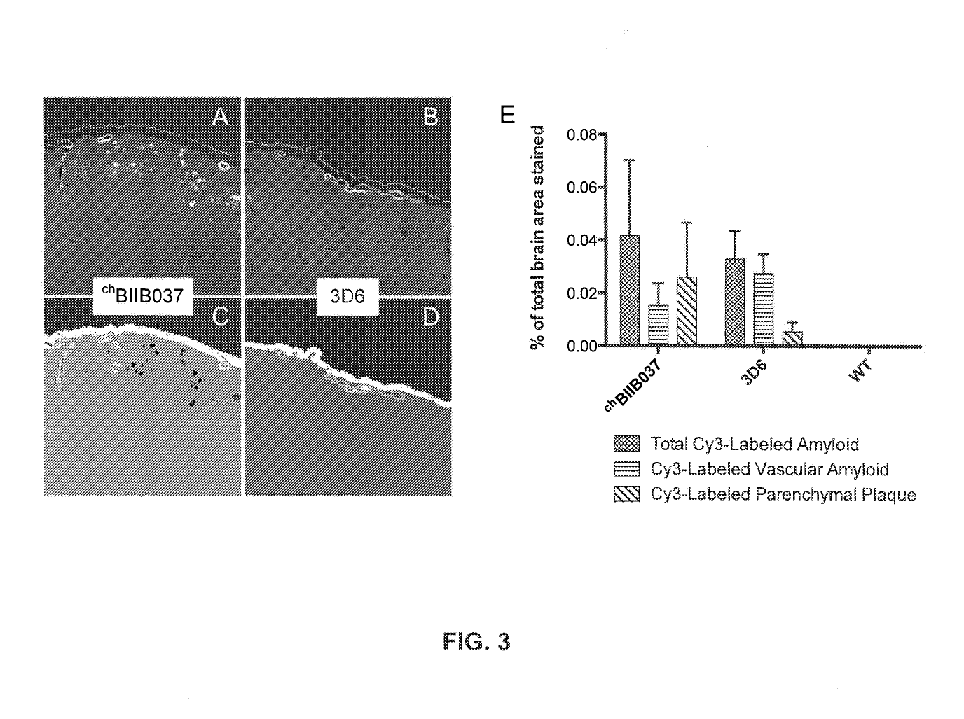

This example further describes the differential chBIIB037 binding. Binding of chBIIB037 to parenchymal amyloid plaques following acute dosing in 22 month-old Tg2576 transgenic mice. Single dose (30 mg/kg) of Cy3-labeled chBIIB037, or Cy3-labeled 3D6, was administered intraperitoneally to Tg2576 transgenic mice, and brains were collected 18 days post-dosing. Frozen brain sections were immunostained for smooth muscle α-actin (SMA) in order to define the blood vessels, and were obtained as described above in Example 2. The binding of the antibody to Aβ deposits was revealed under fluorescent microscope, allowing for direct visualization of the Cy3-chBIIB037 binding to different types of amyloid deposits (FIG. 3A). Cy3-labeled 3D6 antibody was used as comparator for this study (FIG. 3C). The total area of parenchymal and vascular amyloid deposits decorated by the labeled antibodies was determined by quantitative image analysis using an algorithm written with VISIOPHARM® software (Visiopharm A/S, Hoersholm, Denmark). (FIGS. 3B, D and E). Cy3 staining associated with one or the other amyloid compartment was expressed as percent of total staining, and demonstrated preferential binding of chBIIB037 to parenchymal amyloid vs. vascular amyloid (A, B, and E). By comparison, 3D6 preferentially bound to vascular amyloid deposits over parenchymal amyloid plaques (C, D and E). No binding of either chBIIB037 or 3D6 was detected following treatment of wild-type mice (E).

### Example 4

### Exposure Following 6 Months of Chronic Dosing with chBIIB037 in Tg2576 Transgenic Mice

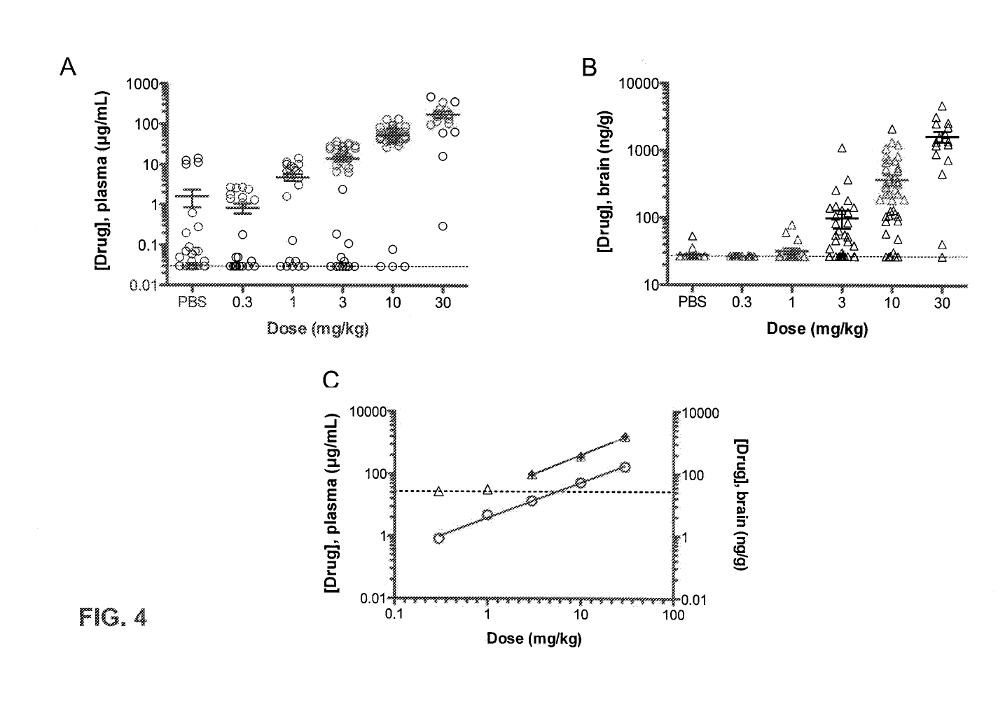

Efficacy of chBIIB037 in reducing amyloid burden was evaluated following 6 months of chronic dosing in Tg2576 transgenic mice. Doses of 0.3, 1, 3, 10, and 30 mg/kg of the antibody, or PBS, were administered weekly via intraperitoneal injection. In order to minimize the generation of a mouse anti-human antibody response, animals were dosed with the murine chimeric version of BIIB037, chBIIB037. Plasma and brain drug levels were measured by ELISA as described above in Example 2. Plasma samples were collected 24 h after the final dose, and chBIIB037 levels measurement showed that exposure correlated linearly with dose (FIG. 4A, 4C), chBIIB037 levels above the limit of detection in the PBS group are due to endogenous anti-Aβ antibodies being measured by the assay. The average brain drug concentrations measured in the diethylamine (DEA)-extracted fraction were proportional to both the administered dosages and to the corresponding plasma drug concentrations (FIG. 4B, 4C). The ratio of the average brain drug concentration to the average plasma concentration for the dose groups at which brain concentrations could be accurately quantitated (3-30 mg/kg) was 0.7-1.0%.

### Example 5

### Reduction of Amyloid Burden Following 6 Months of Chronic Dosing with chBIIB037 in Tg2576 Transgenic Mice

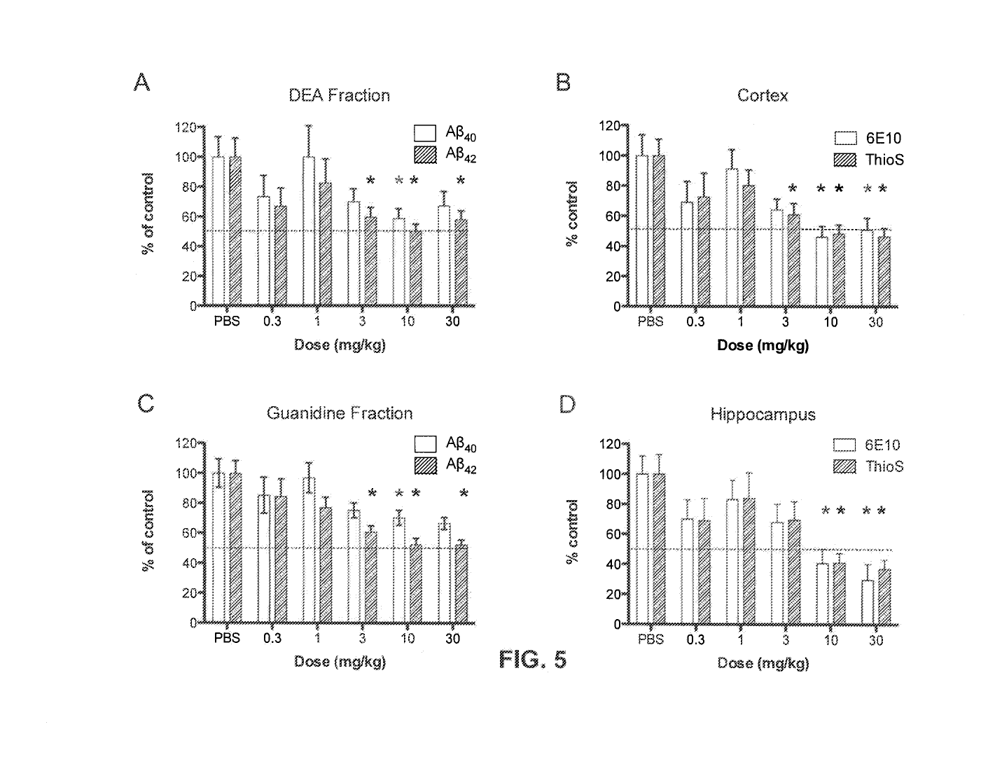

This example describes dose-dependent reductions in brain Aβ load, measured by ELISA using brain homogenate fractions as described above in Example 2. The concentration of individual Aβ species (Aβ1-38 (Aβ38), Aβ1-40 (Aβ40), and Aβ1-42 (Aβ42)) was determined in the DEA fraction, and in the Gu-HCl fraction using the Multi-Spot Human (4G8) Abeta Triplex Assay (Meso Scale Discovery, MD), according to the manufacturer's instructions. Aβ42 concentration was significantly reduced in both soluble DEA (FIG. 5A) and guanidine fractions (FIG. 5C) for the 3, 10, and 30 mg/kg chBIIB037 treatment groups, with percent reductions ranging between 39 and 50% compared to the PBS control. Significant reductions or trends toward reduction of Aβ40 were observed at doses of 3 mg/kg and higher.

Total brain amyloid load in the cortex and hippocampus was revealed by immunohistochemistry. Specifically, brains were dissected and fixed by immersion in 10% neutral buffered formalin for 48 to 72 h. Fixed brains where then processed and embedded in a horizontal orientation. Each block was sectioned until the hippocampus was identified at which point 300 consecutive 5 μm sections (3 sections per slide) were obtained. For both 6E10 and Thioflavin-S staining, every 14^{th} slide was stained (approximately 1 section every 225 μm). Immunohistochemistry to define brain amyloid used mouse anti-Aβ1-16 monoclonal antibody (Clone 6E10, Covance, Princeton, N.J.) as the primary antibody at a 1:750 dilution, and the Ultramap anti-mouse Alkaline Phosphatase Kit (Ventana Medical Systems, Tucson, Ariz.) and quantified using the VISIOPHARM® software as described herein. Slides were pretreated with 88% formic acid solution, prior to being placed on a Ventana Discovery XT immunostainer. Slides were counterstained with Ventana Hematoxylin (Ventana Medical Systems, Tucson, Ariz.), coverslipped and air dried overnight.

After 6E10 immunostaining, slides were scanned with an Aperio XT (Aperio Technologies, Inc., Vista, Calif.) whole slide imaging system at 20× magnification following manufacturer's instructions. Digital images were then reviewed and the best section out of the 3 on each slide was identified. The hippocampus and cortex from this section were manually annotated as separate masks and then analyzed using an algorithm written with VISIOPHARM® software. The algorithm determined the area of the annotated hippocampus or cortex and the areas of parenchymal and vascular amyloid in these anatomic regions at 10× virtual magnification. Training of the software was performed on a set of 50 slides.

Slides were also stained with Thioflavin-S (Thio-S) as described previously (Bussiere et al., Am J Pathol 165:987-995 (2004)), and were coverslipped with VECTASHIELD® Mounting Media with DAPI (Vector Laboratories Burlingame, Calif.). After Thioflavin-S staining, slides were reviewed and the best image out of the 3 sections on each slide was scanned with an Aperio FL (Aperio Technologies, Inc., Vista, Calif.) fluorescent whole slide imaging system at 20× magnification following manufacturer's instructions. As with 6E10, the hippocampus or cortex from this section were manually annotated as separate masks and then analyzed by using an algorithm written with VISIOPHARM® software and adapted for fluorescence. The algorithm determined the area of the hippocampus and the cortex and the areas of parenchymal and vascular amyloid in these anatomic regions at 10× virtual magnification. Training of the software was performed on a set of 10 slides.

The total area occupied by stained deposits was significantly reduced for the 10 and 30 mg/kg doses, with percent reduction ranging between 49 and 70% compared to the PBS control. (FIGS. 5B and 5D). Compact amyloid plaques assessed by Thio-S staining, showed compact plaques were reduced by up to 63% in hippocampus (FIG. 5B), and up to 53% in the cortex (FIG. 5D) at the highest dose of 30 mg/kg.

### Example 6

### The Effect of Chronic Dosing with chBIIB037 on Vascular Amyloid in Tg2576 Transgenic Mice

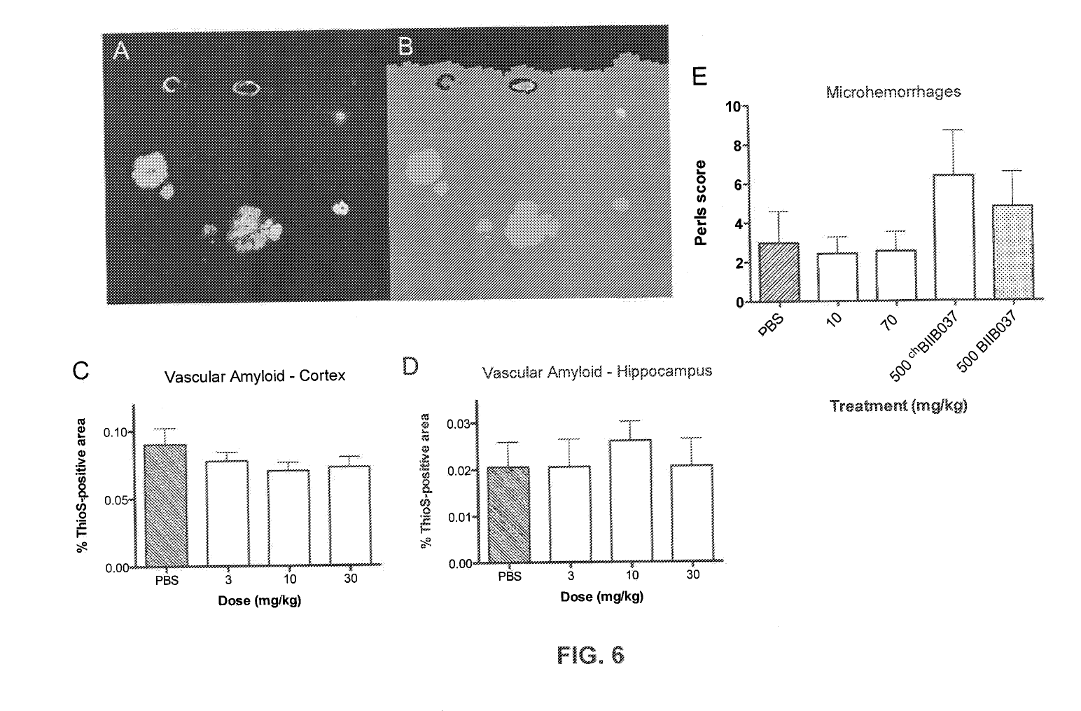

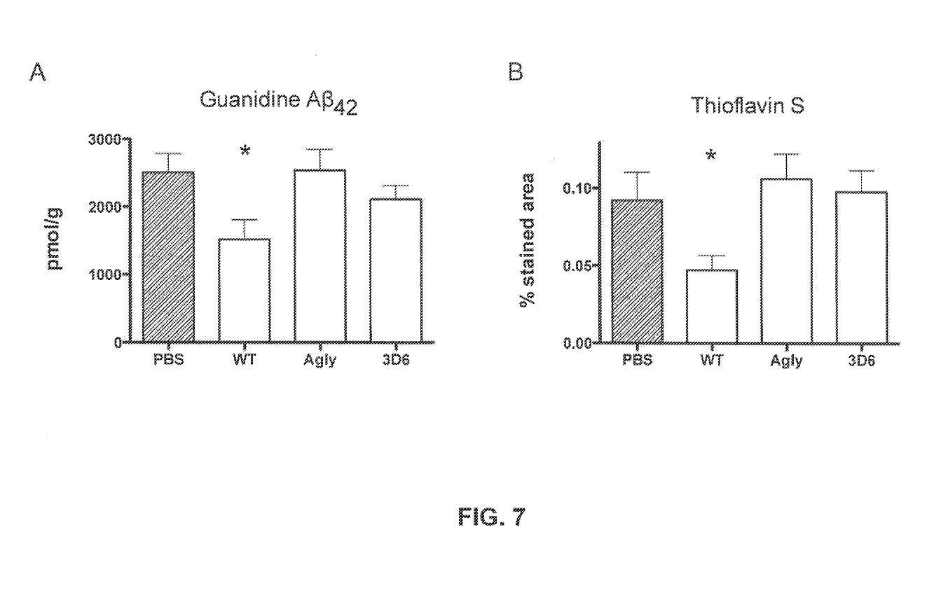

This example examines the effect on vascular amyloid and microhemorrhage following chronic dosing with chBIIB037 in Tg2576 transgenic mice. Thio-S staining of compact amyloid plaques and CAA was revealed under fluorescent microscope (FIG. 6A), and quantified using VISIOPHARM® software (FIG. 6B) as described above in Example 5. Vascular amyloid load in cortex (FIG. 6C) and hippocampus (FIG. 6D) was assessed by Thio-S staining for the 3, 10, and 30 mg/kg doses as described above in Example 5. As demonstrated in FIGS. 6 A-D, chronic dosing with chBIIB037 in Tg2576 transgenic mice had no effect on vascular amyloid load.

Microhemorrhages were assessed in 22 month-old Tg2576 transgenic mice with extensive amyloid pathology in both the parenchyma and the vasculature. Mice were treated chronically for 13 weeks via intravenous (i.v.) treatment of chBIIB037 at 0, 10, 70, 100 or 500 mg/kg (nine to ten mice per sex per group), 500 mg/kg of BIIB037 (ten mice per sex per group), or PBS (control group). At necropsy, brains were harvested, formalin fixed, transversely oriented and trimmed into 5 different blocks spanning through the brain. Three 5 μm tissue sections per block were placed on a slide and stained with Perls stain with a nuclear fast red counter stain. Scoring of microhemorrhage was based on an adaptation of that described previously (Racke et al., J Neurosci 25:629-636 (2005)) except Perls positive profiles had to be identified on two consecutive sections to be scored. As shown in FIG. 6E there were no statistically significant differences in the level of microhemorrhage at the p≦0.05 level between the treatment groups at 10 and 70 mg/kg and the control group. A non-significant trend toward elevated levels was observed upon dose-escalation to 500 mg/kg of chBIIB037 or BIIB037.

### Example 7

### Reduction of Amyloid Burden Following 6 Months of Chronic Dosing with chBIIB037 in Tg2576 Transgenic Mice

This example describes reduction of amyloid burden following 6 months of chronic dosing with chBIIB037 in 10 month-old Tg2576 transgenic mice. Specifically, wild type (wt) chBIIB037 or chBIIB037-Agly were dosed at 3 mg/kg via weekly intraperitoneal injection in Tg2576 mice, and different biochemical or histological endpoints were measured as described above in Examples 2 and 5. An aglycosylated variant of chBIIB037 (chBIIB037-Agly), incorporating a single point mutation (N297Q, using standard. Rabat EU numbering) which eliminates N-glycosylation of the Fe region and severely reduces Fe gamma receptor binding, was generated as described in Tao and Morison, J Immunol. 143:2595-2601 (1989). A group of animals was treated with the same dose of a comparator antibody, 3D6 or PBS control. Brain Aβ₄₂ levels measured in the insoluble (guanidine) fraction prepared from brain homogenates were significantly reduced by up to 55% compared to the PBS-treated control, whereas Aglycosylated (Agly) chBIIB037 and 3D6 had no effect (FIG. 7A) Cortical compact amyloid plaque load quantified by Thio-S staining was significantly reduced in the wt chBIIB037 treatment group, assessed by quantification of area stained with Thio-S as described above in Example 5. chBIIB037-Agly and 3D6 had no effect on the compact amyloid plaque load (FIG. 7B).

### Example 8

### Amyloid Load Reduction Following Chronic Dosing with chBIIB037 in 9.5 Month-Old Tg2576 Transgenic Mice Affects Plaques of all Sizes

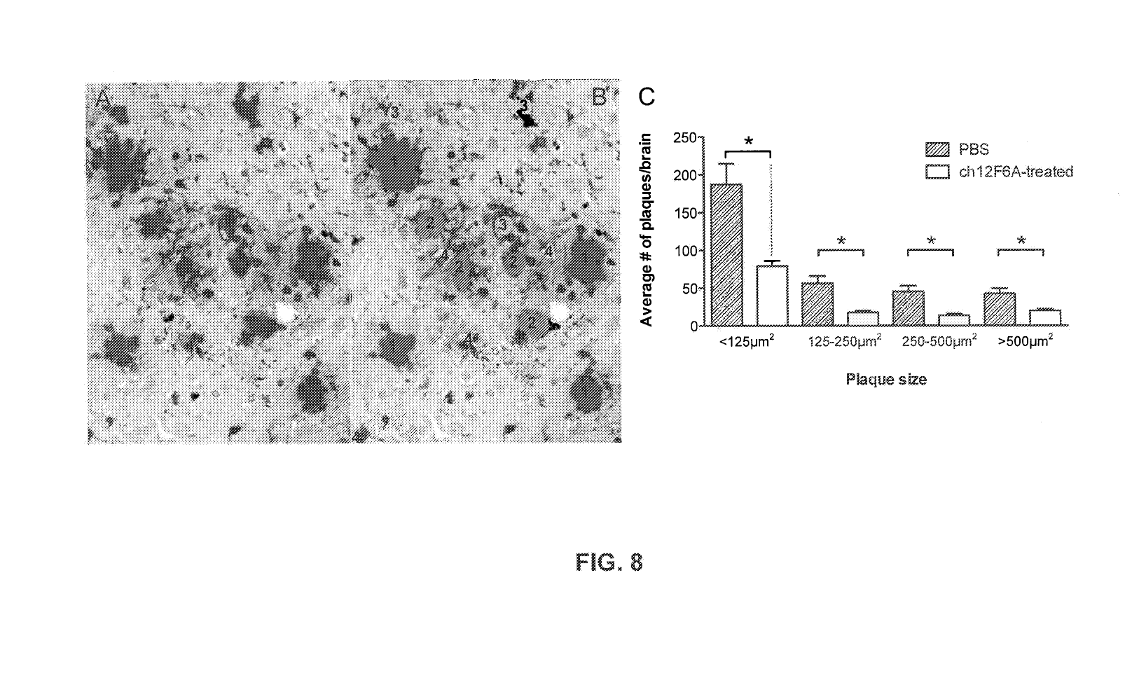

The total amyloid load, including diffuse and compact parenchymal beta-amyloid plaques as well as vascular amyloid or CAA, was revealed by 6E10 immunohistochemistry (FIG. 8A) and quantified using the VISIOPHARM® software (FIG. 8B) as described above in Example 5. The total area occupied by Aβ-stained deposits was significantly reduced for the 10 and 30 mg/kg doses, with percent reduction ranging between 49 and 70% compared to the vehicle control. Trends toward lower amyloid load were observed at the lower doses of 0.3, 1, and 3 mg/kg. Amyloid plaques were classified into 4 categories based on size: plaques <125 μm² (4), 125-250 μm² (3), 250-500 μm² (2) and >500 μm² (1) (FIG. 8B). As shown in FIG. 8C, chBIIB037 treatment was associated with a significant decrease in plaque number in all size ranges compared to PBS-treated controls.

### Example 9

### Microglia-Mediated Clearance of Amyloid Plaques Following Chronic Dosing with chBIIB037 in 9.5 Month-Old Tg2576 Transgenic Mice

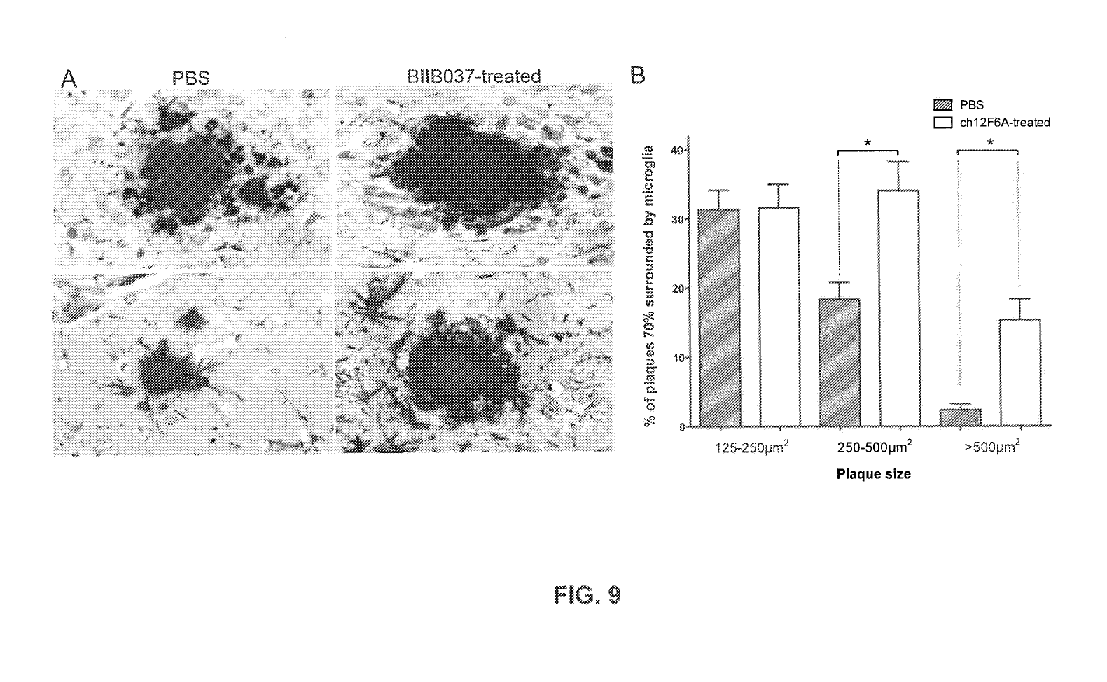

Brain sections from either PBS- or BIIB037-treated mice were immunostained for Aβ (6E10-light gray) and a marker of microglia (Iba1-dark way). Immunostaining with anti-Aβ antibodies 6E10 revealed a different morphology of the amyloid plaques in the chBIIB037-treated groups as compared to the PBS control group. Whereas in control animals, the contour of amyloid plaques is irregular with indistinct “fuzzy” edges, most plaques remaining in the brain after 6 months of treatment consist of a compact dense core with a sharply defined border. (FIG. 9A). Co-immunostaining with activated microglia marker Iba-1 revealed that microglial cells were tightly associated with the compact plaques and often completely surrounded the plaques. These cells were amoeboid in morphology with few ramifications into the neuropil. In contrast, in the PBS-treated control group, microglia rarely surrounded plaques and often presented with abundant ramifications that extended into the neuropil. Analysis using VISIOPHARM® software calculated the area of individual amyloid plaques, and classified Iba1-stained microglia into 2 categories, either surrounding plaques (within 25 μm of a plaque) or not associated with plaques (>25 μm from a plaque). (B) Plaques with circumferences ≧70% surrounded by macrophages were calculated and stratified based on plaque size. Percentage of plaques that are 70% surrounded by microglia was significantly greater in the chBIIB037-treated group (clear bars) compared to the PBS-treated control group (gray bars), for plaques ≧250 μm² (*=p<0.0.005) (FIG. 9B).

### Example 10

### Structural Basis of Aβ Recognition

To understand the molecular basis of antigen recognition, a Fab fragment of BIIB037 in complex with Aβ(1-11) peptide was crystallized. The Fab fragment of BIIB037 was generated by digestion of the full antibody with papain. Briefly, 40 mg of BIIB037 antibody was incubated in digestion buffer (20 mM sodium phosphate, pH7.2, 10 mM EDTA and 20 mM cysteine-HCl) in the presence of 1 mL of immobilized papain beads (Thermo Scientific) at 37° C. for 18 h with rotation mixing. The beads were removed by centrifugation, and the digested antibody solution was dialyzed against phosphate-buffered saline. The solution was then loaded onto a 5 mL Protein A sepharose column to deplete the digested Fc fragment and yield the purified BIIB037 Fab fragment. The BIIB037 Fab was crystallized by the nanodroplet vapor diffusion method at a temperature of 297 K by mixing 200 nL of 7.6 mg/mL BIIB037 Fab solution (10 mM Tris pH 8, 150 mM NaCl) with 200 nL of the reservoir solution containing 19% PEG 3350, 300 mM lithium sulfate, and 100 mM sodium acetate at pH 4. Apo BIIB037 Fab crystals were cross-linked with glutaraldehyde, transferred to a soaking solution containing 19% PEG 3350, 100 mM HEPES pH 7, and 10 mM Aβ1-11 (DAEFRHDSGYE; SEQ ID NO: 9) and soaked for 24 h, prior to harvesting and flash freezing in liquid nitrogen. Diffraction data with an oscillation range of 180° were collected on a FR-E SuperBright copper rotating-anode X-ray generator (Rigaku Americas, Houston, Tex., USA) at 100 K using a Raxis-4++ imaging-plate area detector (Rigaku Americas, Houston, Tex., USA) to measure the reflection intensities. Data were integrated, reduced, and scaled using HKL2000. The BIIB037 Fab:Aβ1-11 crystal was indexed in the monoclinic space group C2. The BIIB037 Fab:Aβ1-11 structure was determined to 2.55 Å resolution by molecular replacement using the apo BIIB037 Fab structure as the search model with the program MolRep. Well-defined electron density was observed for residues Ala2 to Gly9 of Aβ1-11, which was manually built with Coot. No electron density was observed outside of the binding groove that could be modeled for Asp1 or Tyr10 to Glu11 of Aβ. Structure refinement was performed using REFMAC program. The final model includes 1 Fab molecule (chains H and L), Aβ2-9 peptide (Chain Q), and 124 water molecules in the asymmetric unit. The progress of the model refinement was monitored by cross-validation R_{free}, which was computed from a randomly assigned test set comprising 5% of the data. The final R factor is 19.4% with an R_{free} factor of 23.8%. The Ramachandran plot analysis showed that all residues lie within allowed regions.

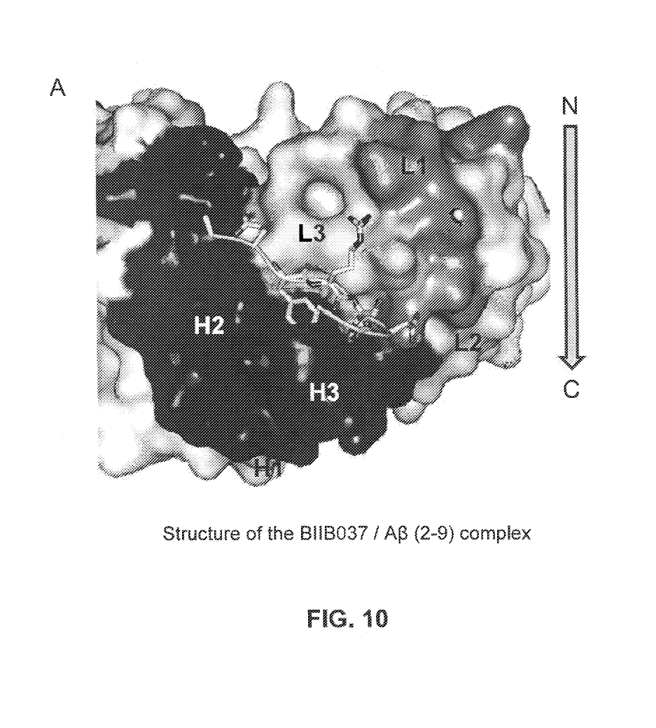

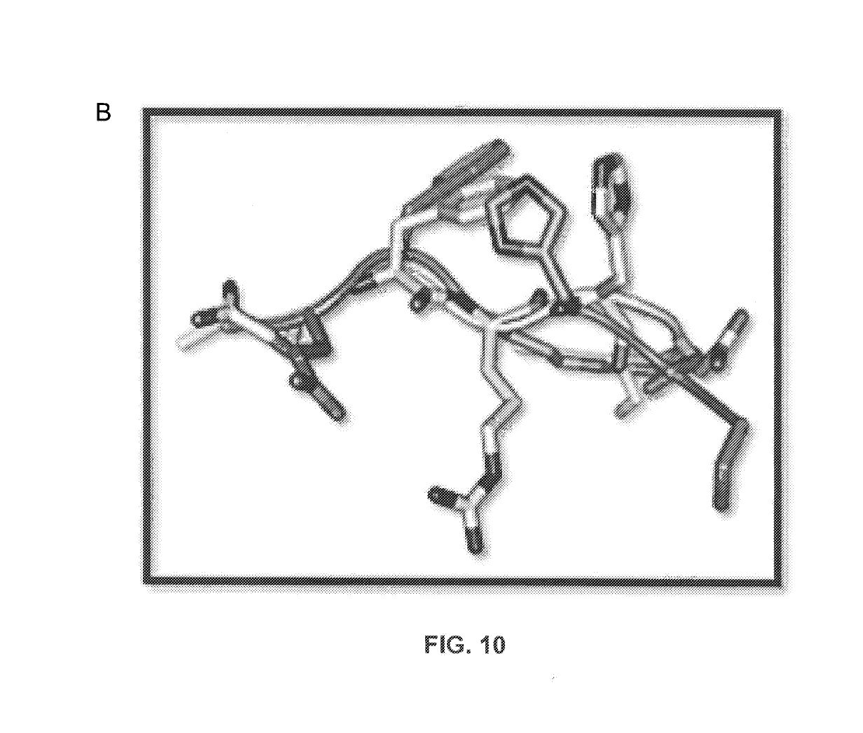

No electron density was observed for AspA or Tyr10 to Glu11 of Aβ. The structure reveals that the Aβ 2-9 adopts a mostly extended conformation stretching 20 Å from the N-terminus to the C-terminus that ends into a short turn composed of Asp7 and Ser8 (FIG. 10A). The primary contact residues on Aβ include Glu-3, Phe-4, Arg-5, and His-6, confirming the epitope mapping data generated using N- and C-terminally truncated peptides. Interactions of the BIIB037 Fab with the Aβ2-9 are found with four of the antigen-binding loops consisting of CDRs L1, L3, H2 and H3. In particular, residues Phe4 and His6 bind within a well-defined pocket within CDRs H2, H3, and L3. The Aβ2-9 peptide has a total surface area of 1157 Δ², of which 46% (530 Δ²) is buried at the antibody interface with high shape complementarity (0.75%).

Amino acids 4-10 have been described as an immunodominant epitope on Aβ (McLaurin, Nature, 8: 1263 (2002); Town, T. et al., Neuroscience Lett., 207(2):101-4 (2001)). Thus, several monoclonal antibodies have been reported to bind to similar linear epitopes within this region of the Aβ sequence. These include: a) a set of antibodies (12A11, 10D5, 12B4, PFA1, PFA2, and WO2) which have a notable degree of sequence similarity within some of the CDRs, and which all bind to Aβ with similar tertiary structures within the Aβ (3-7) region (Basi et al. JBC, (2010)), and b) gantenerumab, which binds to residues 1-11 of Aβ with a different overall structure than the 12A11 family (Bohrmann et al., J. Alz. Disease, 26: 1-21 (2011)). Despite the similar linear epitopes recognized by these antibodies, they display a range of conformational selectivities, including some which do not distinguish between monomeric and aggregated Aβ (e.g., 12B4), and some which bind selectively to aggregated Aβ (e.g. 10D5, 12B4, and gantenerumab). The structure of BIIB037 further differs from both the 12A11 family and gantenerumab, both in the tertiary structure of the antibody-peptide interface, and in the conformation of the bound Aβ peptide itself (FIG. 10B).

### Example 11

### Epitope Mapping of Antibody BIIB037

The epitope for BIIB037 was identified by ELISA using synthetic fragments of the Aβ peptide. For N-terminally truncated Aβ peptides, untagged peptides were used. For the ELISA, peptides were used to coat 96 well microplates at a concentration of 5 microgram/mL in coating buffer (15 mM Na₂CO₃, 35 mM NaHCO₃, pH 9.6) overnight at 4° C. Plates were washed and non-specific binding sites were blocked for 1 h at 25° C. with PBS containing 2% BSA. Antibodies diluted in Assay Buffer (PBS containing 2% BSA) were added, and the plate was incubated for 2 h at 25° C. Binding was determined by incubation with a horseradish peroxidase-conjugated goat anti-human polyclonal antibody (Jackson ImmunoResearch) followed by measurement of HRP activity using the substrate TMB.

For C-terminally truncated Aβ peptides, biotin-conjugated peptides were used. The biotin was attached synthetically to the carboxyl terminus of the protein by incorporation of a non-native lysine residue. For the ELISA, biotin-conjugated peptides were incubated for 1 h at 25° C., at a concentration of 5 microgram/mL in Assay Buffer, with plates pre-coated with Neutravidin (Thermo Scientific Reacti-Bind plates). Plates were washed, antibodies diluted in Assay Buffer were added, and the plate was incubated for 2 h at 25° C. Binding was determined by incubation with a horseradish peroxidase-conjugated goat anti-human polyclonal antibody (Jackson ImmunoResearch) followed by measurement of HRP activity using the substrate TMB.

As shown in Table 4N-terminal truncations beyond residue Glu-3 of Aβ, and C-terminal truncation beyond residue His-6, led to significant losses in affinity. These results indicate that the primary binding site for BIIB037 comprises residues 3-6 of human Aβ.

TABLE 4
Binding of BIIB037 to synthetic Aβ peptide fragments.

| Peptide | EC₅₀ (nM) |
|---|---|
| 1-42 | 0.4 |
| 2-42 | 0.4 |
| 3-42 | 0.5 |
| 4-42 | 15 |
| 5-42 | 35 |
| 6-42 | >100 |
| 8-42 | >100 |
| 1-5-Lys-biotin | >100 |
| 1-6-Lys-biotin | 0.3 |
| 1-7-Lys-biotin | 0.2 |
| 1-8-Lys-biotin | 0.2 |
| 1-9-Lys-biotin | 0.5 |
| 1-12-Lys-biotin | 0.5 |
| 1-16-Lys-biotin | 0.5 |

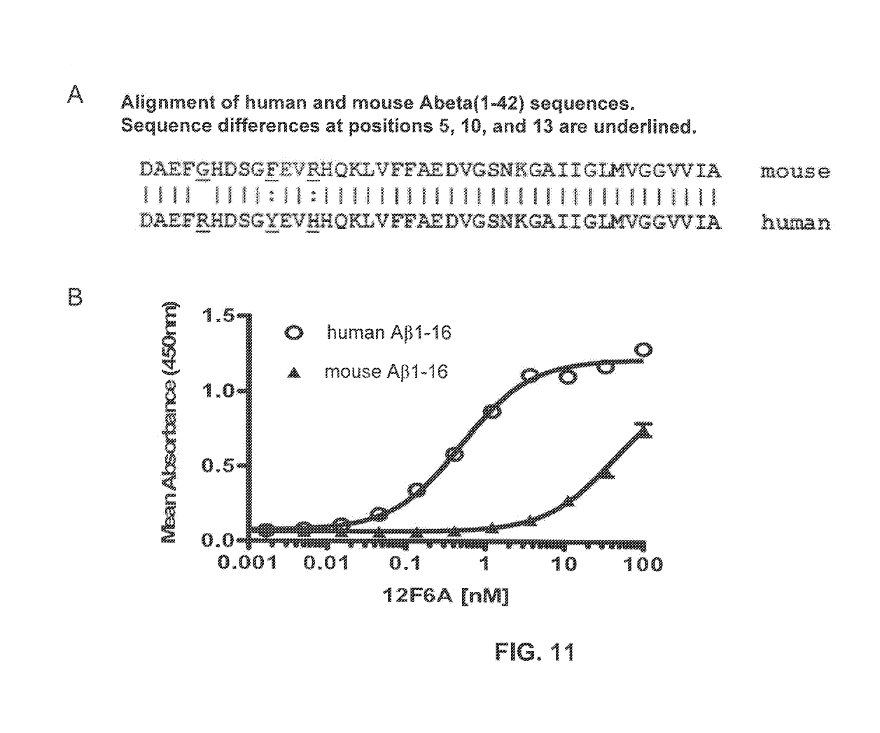

The epitope for BIIB037 was further investigated by measuring the relative binding to human and mouse Aβ peptides. The human and mouse Aβ peptide sequences differ by three amino acids, at positions 5, 10, and 13 (FIG. 11A). Biotinylated synthetic peptides corresponding to the Aβ (1-16) sequence were used to measure binding of BIIB037 by ELISA, as described above (FIG. 11B). This peptide contains all three differences between the human and mouse sequences, but within the BIIB037 epitope (Aβ residues 3-6, established through binding studies with peptide fragments) there is a single amino acid difference, at position 5. The EC₅₀ values for human and mouse Aβ were 0.5 and 43 nM, respectively. The 90-fold decrease in binding EC₅₀ on the mouse peptide indicated that BIIB037 binds selectively to human Aβ, and that residue Arg-5 is a critical determinant of binding.

### Example 12

### Overview of SPECT Imaging Data in Tg2576 and WT Mouse CNS Using [¹²⁵I]chBIIB037, [¹²⁵I] Agly-chBIIB037, and [¹²⁵I] Human BIIB037 F(ab′)2′

Single Photon Emission Computed Tomography (SPECT) imaging was employed to track the entry of murine chimeric and human variants of BIIB037 antibody into brains of wild type (WT) and Tg2576 (Tg) mice. Iodination of BIIB037, Agly-chBIIB037 and human BIIB037 F(ab′)2 was carried out using a modified version of the Iodogen method (Thermo-Scientific/Pierce Biotechnology, Rockford, Ill.). Briefly, 600 μg of chBIIB037 or Agly-chBIIB037 was treated with 20 mCi ¹²⁵I-NaI in 4 Pierce Iodination tubes pre-coated with 50 μg of Iodogen (Pierce Iodination Reagent; 150 μg chBIIB037, Agly-ch12F6A or human BIIB037 F(ab′)2 and 5 mCi ¹²⁵I-NaI per tube) and Tris Iodination Buffer (25 mM Tis-HCl, 0.4 mM NaCl pH7.5 and giving a total incubation volume of 200 μl). After gentle agitation of the tubes at room temperature for 9 min 500 of scavenging buffer (10 mg/ml Tyrosine in Tris Iodination buffer) was added and the mixture gently agitated for another 5 min followed by addition of 1 ml 25 mM Tris-HCl, 0.4 mM:NaCl pH7.5, 5 mM EDTA, 0.5% sodium azide to each of the 4 tubes. Subsequently the combined products from the 4 tubes were added to Centricon 30K ultrafiltration units and centrifuged at 6000 rpm for 5 min. The columns were then washed with 4×1 ml saline, 10 μl of sample was diluted to 0.5 ml for OD measurement and 100 μl sample was diluted with 1 ml for HPLC analysis. The remainder was available for the study. In these studies, 5 mCi of [¹²⁵I] was used to label 0.15 mg chBIIB037 or Agly-chBIIB037 at approximately 80% labeling efficiency ultimately resulting in approximately 1.8 iodines per antibody. The activity of the labeled antibody was confirmed by generating a sample labeled with non-radioactive iodine in parallel, under identical conditions, and measuring binding affinity for Aβ using an ELISA.

22-month old female Tg2576 (Tg, n=14) and non-transgenic C57/BL/6. (WT, n=14) mice were administered carrier added [¹²⁵I]chBIIB037 (1.25-1.5 mCi), 10 mg/kg 0.1 ml via tail vein injection on days 0, 7, 14, and 21 as highlighted in Table 5. Mice were given drinking water containing potassium iodide (0.2 g/L) to reduce uptake of free iodine in the thyroid. SPECT/computed tomography (CT) imaging was carried out just prior to subsequent injections of radiolabeled antibody on days 7 and 14 and also on days 28, 48, and 49 as highlighted in Table 5. For SPECT/CT imaging, mice were anesthetized with 2-3% isofluorane in 100% oxygen. The scans were performed using a small animal dedicated NanoSPECT/CT camera (Bioscan/Mediso) equipped with 1.4 mm diameter aperture pinhole collimators (×9). Batch processed reconstruction of the SPECT data was carried out by inviCRO LLC (Boston, Mass.) and analyzed utilizing a proprietary mouse brain atlas analysis protocol combined with CT co-registration of the SPECT reconstructions. Animals 103 and 111 (Tg) and animals 205 and 210 (WT) were also administered [¹¹¹In]chBIIB037 at the end of the iodine-labeled study as a preliminary comparison of using a clinically accepted radioisotope in place of the radio-iodine (Table 5).

TABLE 5
Key to summary data for imaging study and post-study ex-vivo assessment

| Mouse | | | Day | Day | Day | Day | | |
|---|---|---|---|---|---|---|---|---|
| Identification # | Gp | Day 7 | 14 | 28 | 48 | 49 | ¹¹¹In | Ex-vivo Analysis |
| 101 | Tg | X | X | X | | | | mARG, BioD |
| 102 | Tg | X | | | | | | mARG |
| 103 | Tg | X | X | X | X | | X | Whole Head ARG |
| 104 | Tg | X | X | | | | | None (Died) |
| 105 | Tg | X | X | X | X | X | | ½ brain frozen, ½ fixed for BIIB |
| 106 | Tg | X | X | X | | | | mARG, BioD |
| 107 | Tg | X | X | X | | | | PARTI, BioD |
| 108 | Tg | X | X | X | X | X | | ½ brain frozen, ½ fixed for BIIB |
| 109 | Tg | X | X | X | X | X | | ½ brain frozen, ½ fixed for BIIB |
| 110 | Tg | X | X | | X | X | | ½ brain frozen, ½ fixed for BIIB |
| 111 | Tg | X | X | X | X | | X | PARTI |
| 112 | Tg | X | X | | | | | PARTI |
| 113 | Tg | X | X | X | X | X | | ½ brain frozen, ½ fixed for BIIB |
| 114 | Tg | X | X | X | X | X | | ½ brain frozen, ½ fixed for BIIB |
| 201 | WT | X | X | X | | | | mARG, BioD |
| 202 | WT | X | | | | | | PARTI |
| 203 | WT | X | X | X | | | | PARTI, BioD |
| 204 | WT | X | | | | | | mARG but NOT used |
| 205 | WT | X | X | X | X | | X | None (Died) |
| 206 | WT | X | | | | | | mARG |
| 207 | WT | X | X | X | X | X | | ½ brain frozen, ½ fixed for BIIB |
| 208 | WT | X | X | X | X | X | | ½ brain frozen, ½ fixed for BIIB |
| 209 | WT | X | | X | X | | | No ex-vivo analysis |
| 210 | WT | X | | | X | | X | Whole Head ARG |
| 211 | WT | X | | | X | | | No ex-vivo analysis |
| 212 | WT | X | X | X | X | | | Not Imaged |
| 213 | WT | X | X | X | X | | | Not Imaged |
| 214 | WT | X | X | X | X | | | Not Imaged |

mARG—microautoradiography;
BioD—Biodistribution;
PARTI—Patho Auto Radio Tomography Imaging

Batch processed reconstruction of the SPECT data was carried out by inviCRO LLC (Boston, Mass.) and analyzed utilizing a proprietary mouse brain atlas analysis protocol combined with CT co-registration of the SPECT reconstructions.

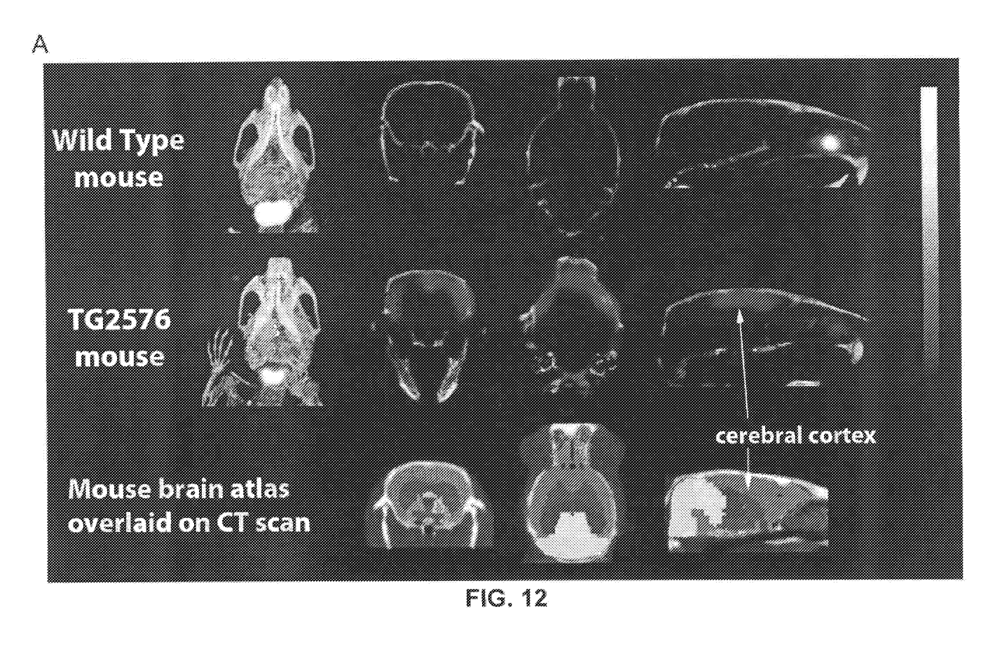

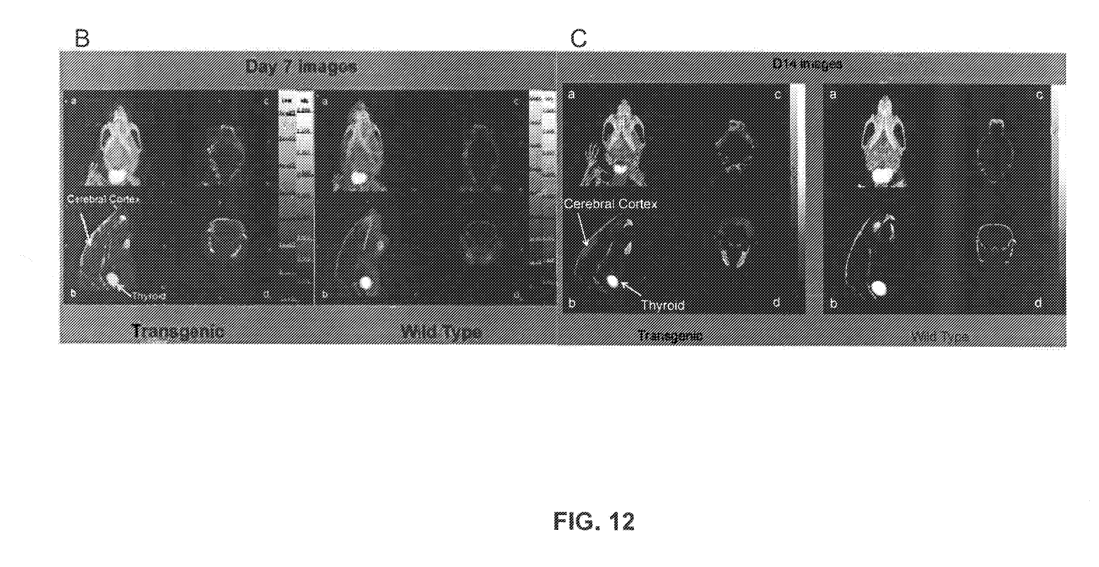

In vivo [¹²⁵I]chBIIB037 antibody uptake was assessed in the brain of Tg2576 and WT mice (n=14 animals/group). Overview SPECT images of the averaged data from all animals in the study are highlighted in FIG. 12A and show in vivo cortical uptake of [¹²⁵I]chBIIB037 in Tg2576 but not wild type mice. Acquisition data obtained following 7, 21, 28 and 48 days post-intravenous administration of [¹²⁵I]chBIIB037 antibody showed a time- and cumulative dosing-dependent accumulation of radioactivity in the CNS of Tg2576 mice compared to WT controls (FIGS. 12B-E). The data are presented either as percent of injected dose per gram tissue (% ID/g), concentration (μCi/mm³) or as a ratio of concentration in the region of interest normalized to concentration in cerebellum (null tissue). The amount of radioactivity in the brain over the course of the study ranged from 2 to 10 nCi/mm³ across all regions.

FIGS. 12 B-C represent the cumulative in vivo incorporation of [¹²⁵I]chBIIB037 with similar scaling into Tg2576 mouse cortex shown in the sagittal (b), horizontal (c) and coronal (d) plane on day 7 (FIG. 12B) compared to day 14 (FIG. 12C) of the study and following two i.v. injections of 1.5 mCi radiolabel. Image (a) in each panel represents the CT image used to co-register SPECT images and the brain atlas (inviCRO LLC) for subsequent quantification of the accumulation of radioactivity Representative day 7 and day 14 SPEC I′ imaging with high levels of uptake of [¹²⁵I]chBIIB037 in Tg2576 (left) and WT (right) mouse show in vivo uptake in cortical regions of Tg2576 but not W f mouse brain.

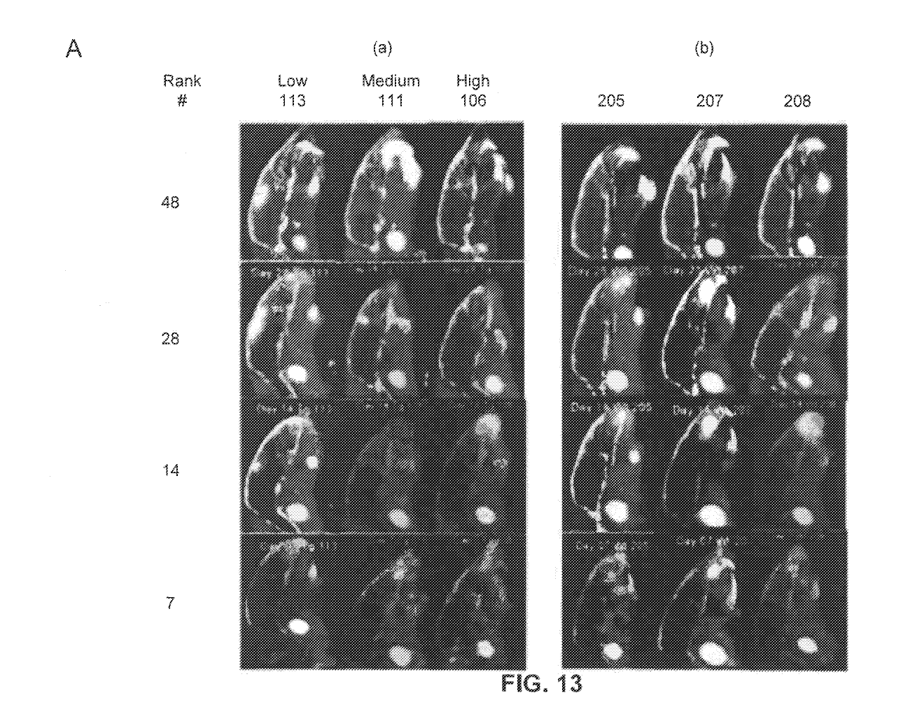

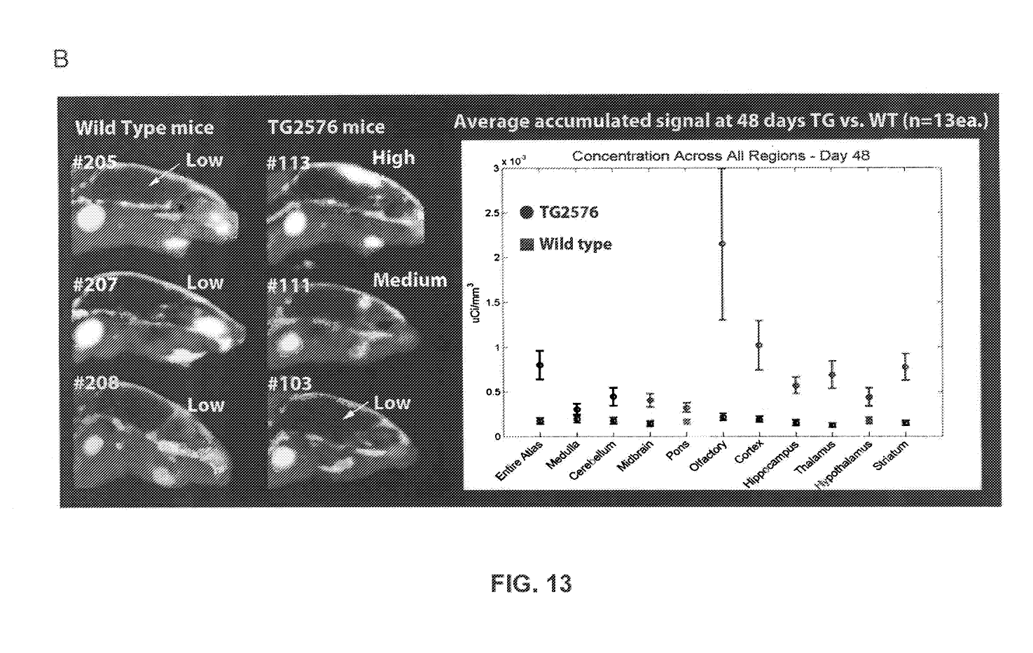

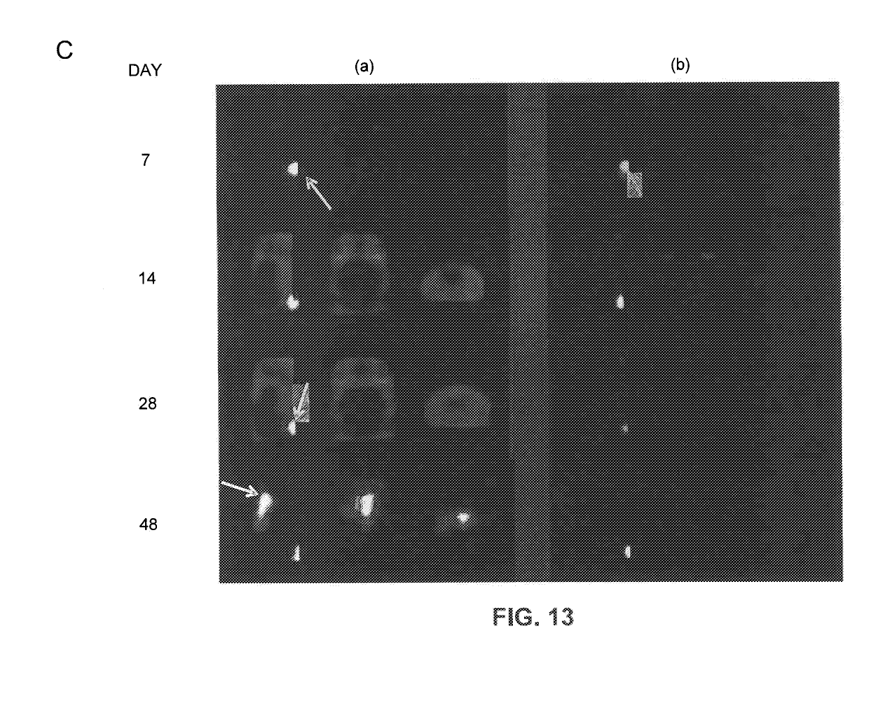

Accumulation of [¹²⁵I]chBIIB037 in Tg2576 mice could be classified (arbitrarily) into three groups as high, medium and low levels of uptake (FIG. 13). FIGS. 13A-B show in vivo uptake and accumulation of [¹²⁵I]chBIIB037 into the CNS compartment of Tg2576 mice over time that is absent from WT mice and that can be ranked into mice accumulating low, medium and high concentrations of the radiolabel. FIG. 13C represents averaged normalized uptake of [¹²⁵I]chBIIB037 in mouse brain over 48 days following repeated administration of radiolabel every 7 days for 4 weeks. Arrows shown on day 7 in Tg and WT mouse, and day 28 in Tg mouse indicate uptake of labeled free iodine into thyroid. Arrow shown on day 48 in Tg mouse indicates uptake into CNS compartment of Tg2576 (a) mice with no uptake into WT control brain (b).

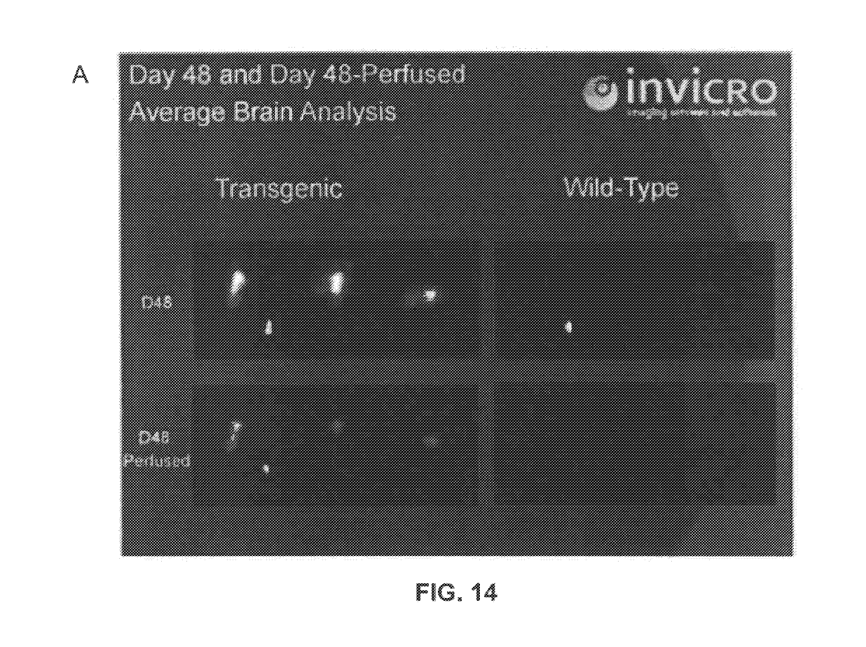

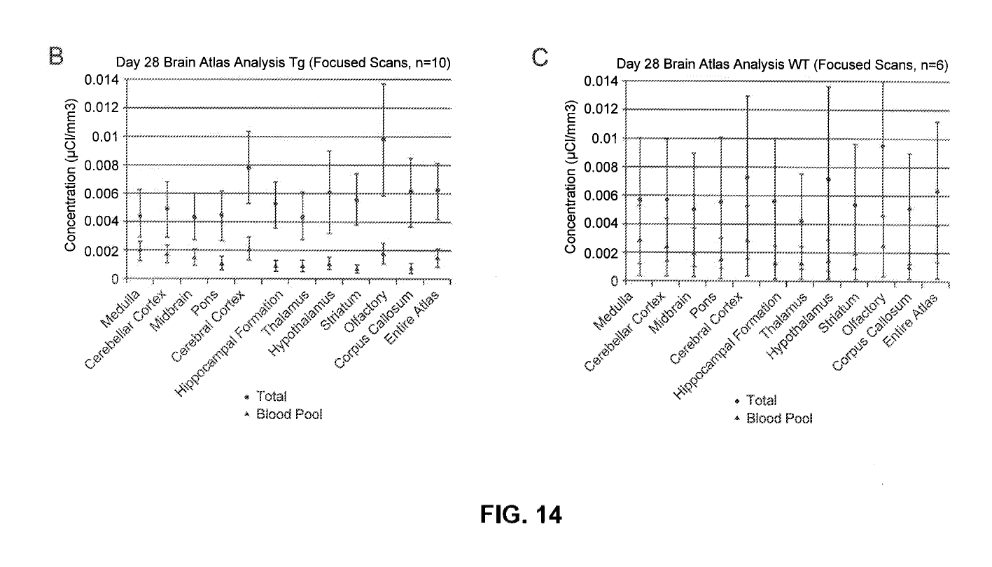

FIG. 14 shows in vivo uptake of [¹²⁵I]chBIIB037 in Tg and WT mouse brain versus blood pool. Top panel shows average brain uptake in Tg2576 (L) versus WT (R) mice at 48 days (D48) and then compared following perfusion with PBS (D48 Perfused). Lower panel shows region-of-interest (ROI) generated plots from the data at day 28 and indicates that uptake in the brain of Tg2576 mice is not due to blood pool as is the case in WT mice where there is no significant difference in the measured concentration of radioactivity (mCi/mm³) between blood pool and tissue.

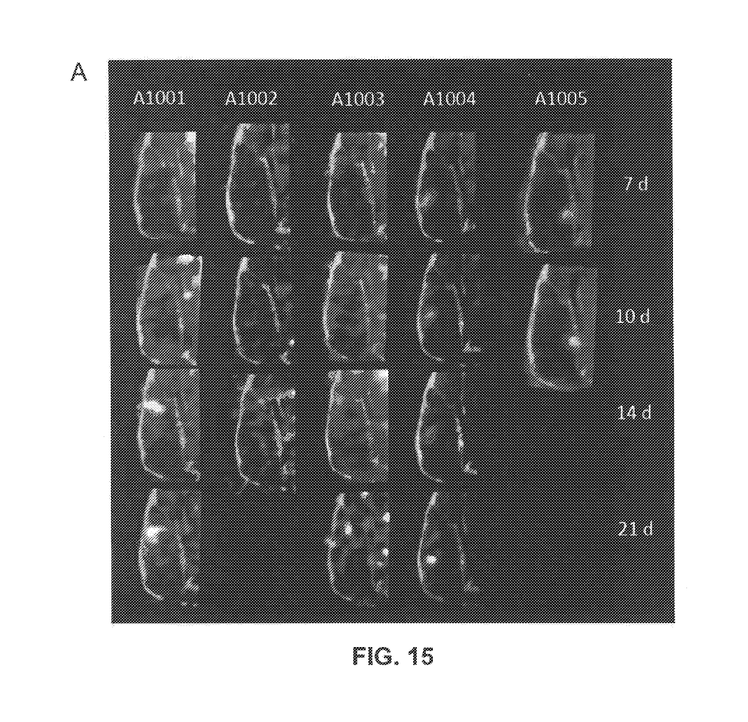

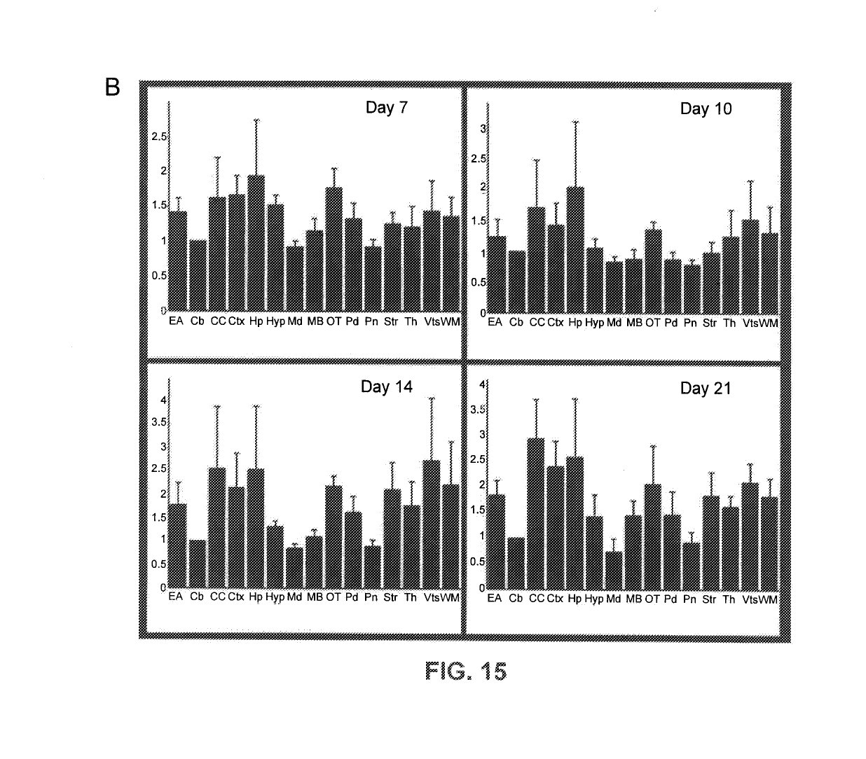

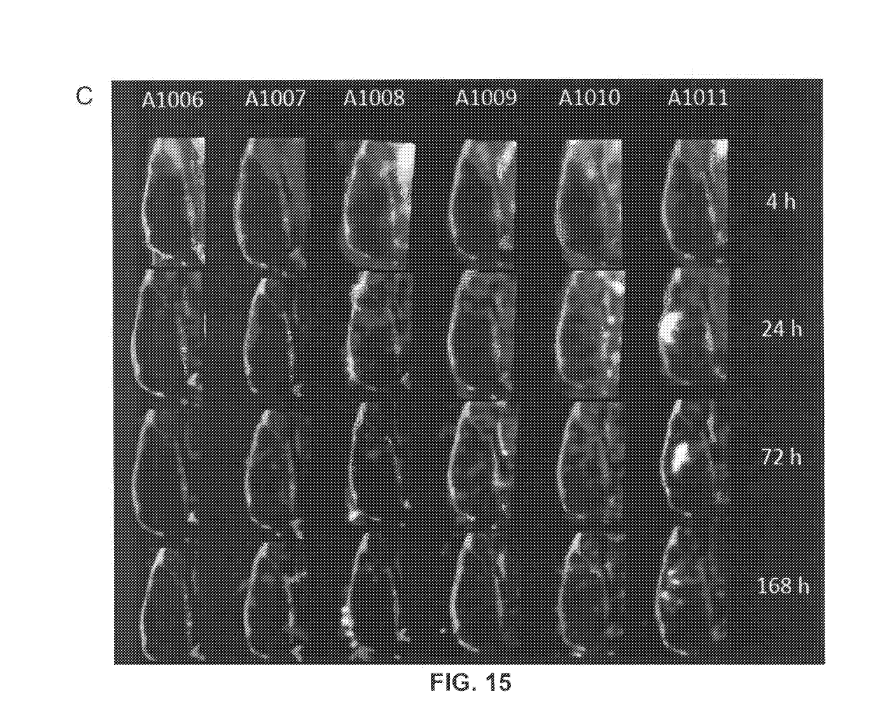

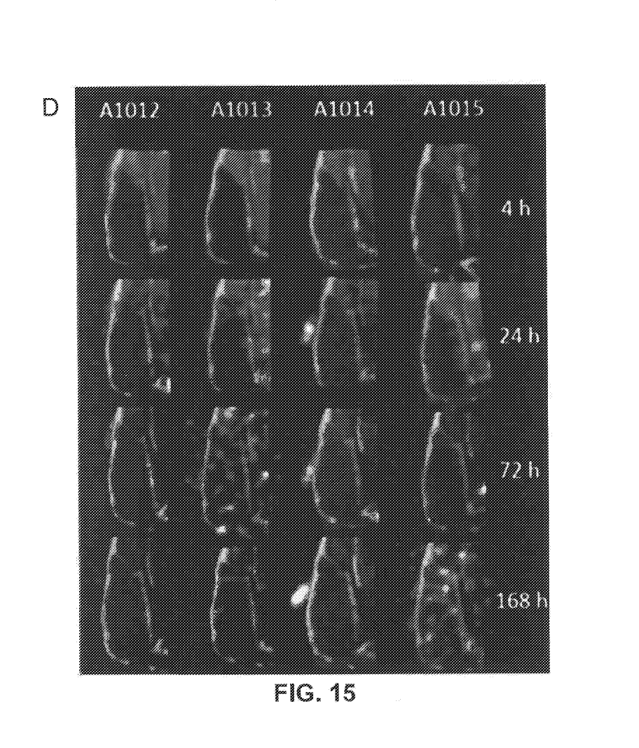

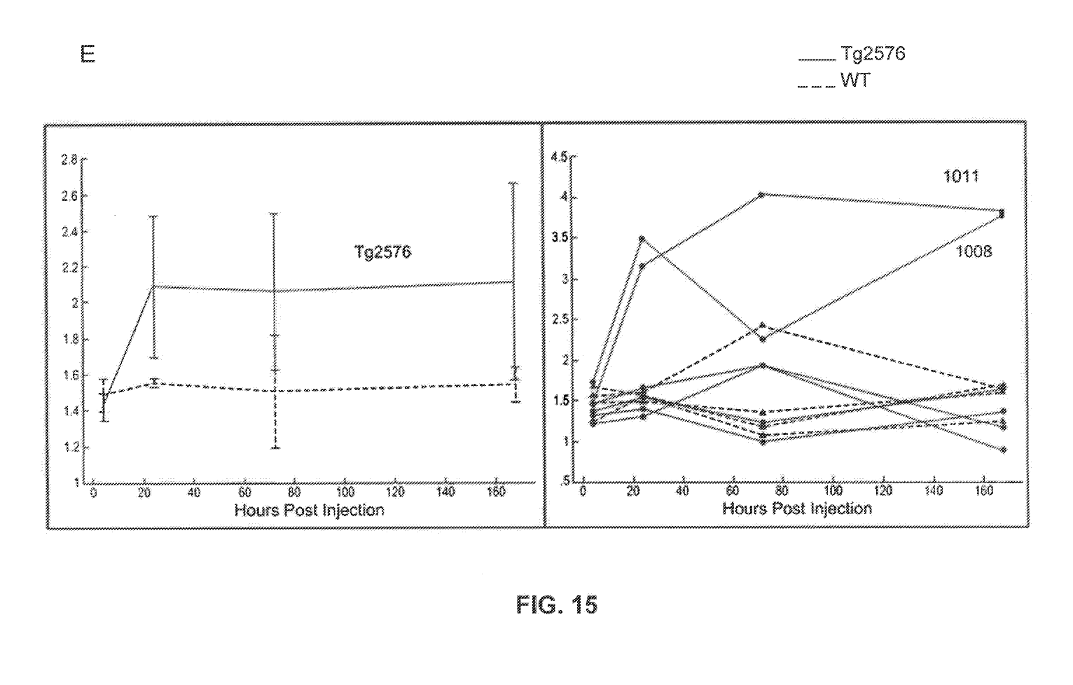

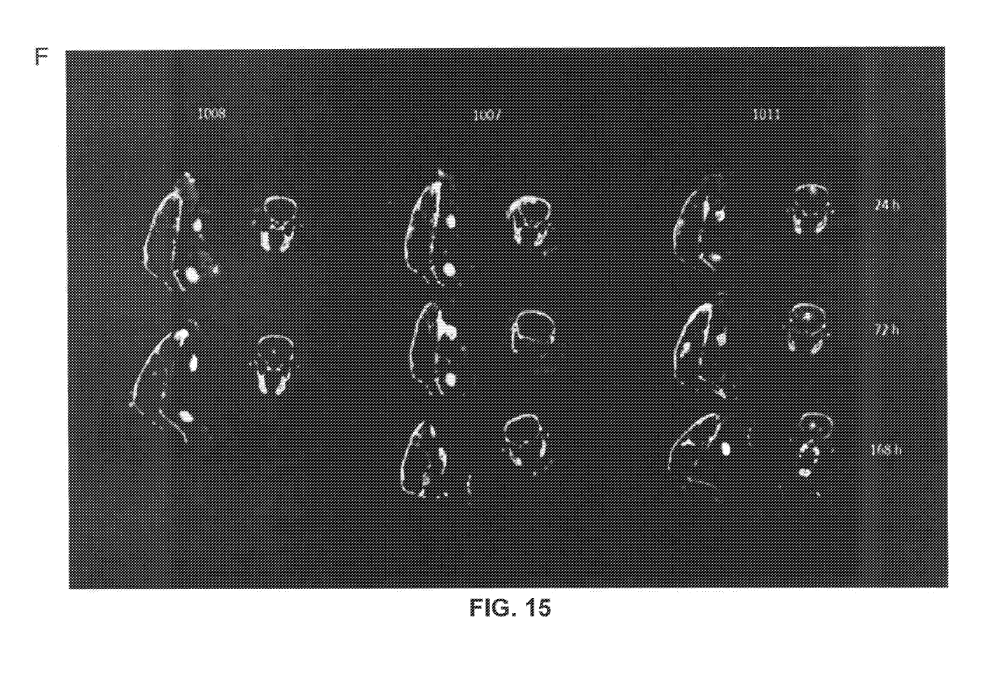

FIGS. 15-18 represent in vivo SPECT imaging with [¹²⁵I]Agly-chBIIB037 and [¹²⁵I]huBIIB037 F(ab′)2. Specifically, 22-month (n=15) old female Tg2576 (Tg) and 3-month (n=4) old female WT mice were administered carrier added [¹²⁵I]Agly-chBIIB037 (1.3 mCi) at 1 mg/kg (n=5) or [¹²⁵I]huBIIB037 F(ab′)2 at 1 mg/kg (n=7 Tg and 4 WT) in a volume of 0.1 ml via tail vein injection on day 0 (Table 6). Mice were given drinking water containing potassium iodide (0.2 g/L) to reduce uptake of free iodine in the thyroid. SPECT/CT imaging was carried out on days 7, 10, 14 and 21 for [¹²⁵I]Agly-chBIIB037 as highlighted in Table 6. For [¹²⁵I]huBIIB037 F(ab′)2 SPECT/CT imaging was carried out at 4, 24, 72 and 168 h post i.v. administration of radiolabeled material (Table 6). In all cases mice were anesthetized and the scans were performed as described above.

TABLE 6
Key to summary data for image acquisition
Study Design ¹²⁵I-material

| Group/ | Females, | Imaging agent | i.v. timeline | Radioactive | Scan |
|---|---|---|---|---|---|
| Route | n (age) | (Dose) | (day) | Dose (mCi) | Timeline (day/hr) |
| 1 | 5 Tg | [¹²⁵I]Agly-chBIIB037 | 0 | 1.3 | 7, 10, 14, 21 day |
| | (22 month) | (1 mg/kg) | | | |
| 2 | 3 Tg | [¹²⁵I]Agly-chBIIB037 | 0 | 1.4 | 4, 24, 72, 168 hr |
| | (22 month) | (1 mg/kg) | | | |
| 3 | 4 Tg | [¹²⁵I]huBIIB037 | 0 | 1.3 | 4, 24, 72, 168 hr |
| | (22 month) | F(ab′)2 | | | |
| | | (1 mg/kg) | | | |
| 4 | 2 WT | [¹²⁵I]huBIIB037 | 0 | 1.5 | 4, 24, 72, 168 hr |
| | (3 month) | F(ab′)2 | | | |
| | | (1 mg/kg) | | | |
| 5 | 2 WT | [¹²⁵I]huBIIB037 | 0 | 1.4 | 4, 24, 72, 168 hr |
| | (3 month) | F(ab′)2 | | | |
| | | (1 mg/kg) | | | |

For groups 2 and 4, focused brain SPECT scans were performed for approximately 30 min followed by a CT scan at the following time points: 4, 24, 72, and 168 hours post-injection. For groups 3 and 5, whole body SPECT scans were performed for approximately 30 min followed by focused brain SPECT scans for approximately 30 min followed by a CT scan at the following time points: 4, 24, 72, and 168 h post-injection. Following scanning, whole body activity was counted using a dose calibrator. After the day 14 or hour 168 imaging session the animals were sacrificed. Prior to imaging, inviCRO ran a resolution, calibration and quantification study on the system as a quality control measure. 13 mice were chosen for imaging.

The radiochemical purities of the ¹²⁵I-labeled Agly-chBIIB037 and huBIIB037 F(ab′)2 are presented in Table 7.

TABLE 7
Radiochemical purities of labeled Agly-chBIIB037 and huBIIB037 F(ab′)2
Radiolabeling purity for [¹²⁵I] labeled antibodies

| [¹²⁵I]Agly-BIIB037 | [¹²⁵I]huBIIB037 F(ab′)2 |
|---|---|
| 94.07 | 99.9 |

Apparent specific activity and molar ratio of [¹²⁵I] labeled antibodies

| | Apparent | | |
|---|---|---|---|
| | specific | Apparent | |
| | activity | specific activity | Molar ratio |
| Antibody | (mCi/mg) | (Ci/mmol) | (Isotope/mAb) |
| [¹²⁵I]Agly-chBIIB037 | 38.13 | 5717.96 | 2.63 |
| (Tg, 1 mg/kg) | | | |
| [¹²⁵I]huBIIB037 F(ab′)2 | 42.20 | 6330.61 | 2.91 |
| (Tg, 1 mg/kg) | | | |
| [¹²⁵I]huBIIB037 F(ab′)2 | 20.24 | 3036.67 | 1.40 |
| (WT, 1 mg/kg) | | | |

In vivo [¹²⁵I]Agly-chBIIB037 uptake was assessed in the brain of 22 month old Tg2576 mice (n=5). Images of the averaged data from all animals in group 1 show in vivo uptake and retention of radiolabel in CNS of 1 mg/kg [¹²⁵H]Agly-chBIIB037 (FIG. 15A). Data represent sagittal images from focused head scans over the duration of the study co-registered with CT scans and normalized to the 99^{th} percentile of each image. Animals 1001-1003 were classified as medium uptake animals and animal 1004 was classified as having high uptake and retention of radiolabeled biologic. Animal 1005 was classified as low uptake based on the probability that the signal observed is coming from radiolabel in the 3^{rd} ventricle.

Region-of-interest (ROI) analysis of data represented in FIG. 15A over each time point revealed a maximal signal:noise ratio of approximately 2.5 in corpus callosum, cortex, hippocampus, ventricles, olfactory bulb and white matter averaged over data from 5 animals at day 21, compared to a target/cerebellum ratio of approximately 2.0 for striatum and thalamus. Other regions analyzed had a ratio of less than 1 (FIG. 15B).

Focused brain scans of the in vivo uptake of [¹²⁵I]huBIIB037 F(ab′)2 in groups 2 and 3 were assessed in six animals. FIG. 15C represents sagittal images from focused head scans over duration of study co-registered with CT scans and normalized to the 99^{th} percentile of each image. The data shows uptake and retention of [¹²⁵I]huBIIB037 F(ab′)2 in aged Tg2576 mouse brain. Animals 1006-1010 are classed as low/no uptake animals except for 1008 which was classed as a low/medium uptake. Animal 1011 is classed as having high uptake and retention of radiolabeled biologic. In this animal a high retention was observed for as long as 168 h post-administration of the radiolabel.

Uptake and retention of [¹²⁵I]huBIIB037F(ab′)2 was measured in young (3 months) WT mouse brain. FIG. 15D represents sagittal images from focused head scans over duration of study co-registered with CT scans and normalized to the 99^{th} percentile of each image to more clearly define classification as low or high retention. Young WT mice shows low/no uptake and retention of [¹²⁵I]huBIIB037 F(ab′)2.

The classification of animals 1008 and 1011 was further confirmed by ROI quantitation highlighted in FIG. 15E. Left hand panel shows averaged data represented as % ID/g in cortex normalized to that in cerebellum over four imaging time points and shows higher retention in cortex of Tg2576 mouse brain compared to WT mouse brain. Right hand panel represents individual uptake data and highlights the two Tg2576 mice, 1008 and 1011, that had a higher retention of [¹²⁵I]huBIIB037 F(ab′)2 compared to that of WT (red lines) and the remaining four Tg2576 (black lines) mice.

Representative images were selected to show in vivo uptake and retention of [¹²⁵I]huBIIB037F(ab′)2 in Tg2576 mice. FIG. 15F represents sagittal and coronal images from focused head scans over the duration of the study co-registered with CT scans and arbitrarily scaled for each animal to reduce background signal in order to more clearly visualize spatial localization of radioactivity. Animal 1011 shows relatively high accumulation and retention of radioactivity in the brain from 2 hrs through 168 hrs. Animal 1008 displayed a similar though lower quantifiable cortex to cerebellum ratio across the duration of the study compared to animal 1011 and again shows some uptake and retention. In contrast, animal 1007 showed the least quantifiable signal and under this arbitrary scaling also shows no significant uptake or retention of radiolabel.

FIG. 15G shows overall summary of quantitative region of interest analysis of in vivo [¹²⁵I]huBIIB037 F(ab′)2 binding in aged Tg2576 and wild type mouse brain, Data represented as % ID/g in cortex normalized to that in cerebellum over four imaging time points shows higher retention of [¹²⁵I]huBIIB037 F(ab′)2 in cortex, hippocampus and striatum of Tg2576 (middle panel) mouse brain compared to WT (lower panel) mouse brain. Top panel confirms previous data showing in vivo accumulation of [¹²⁵I]Agly-chBIIB037 in the same regions.

FIG. 16 shows representative day 48 images showing in vivo accumulation of [¹²⁵I]Agly-ChBIIB037 (1 and 10 mpk) into CNS of Tg2576 aged mouse brain. CNS-specific in vivo accumulation is not observed in young Tg2576 mouse brain using [¹²⁵I]P1.17 labeled isotype control (10 mpk).

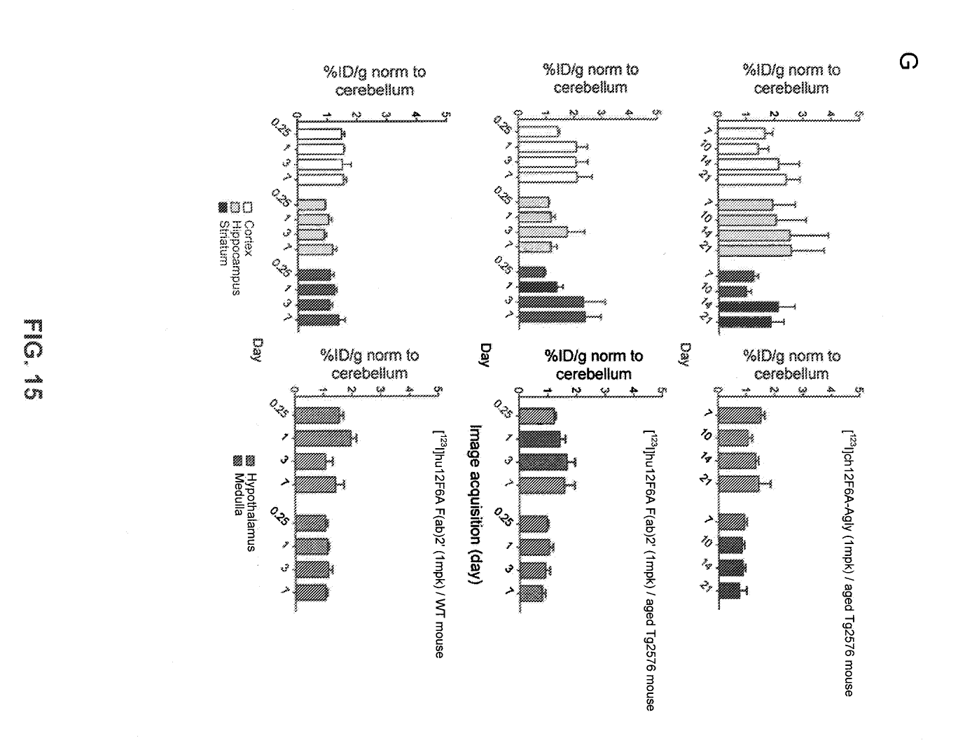

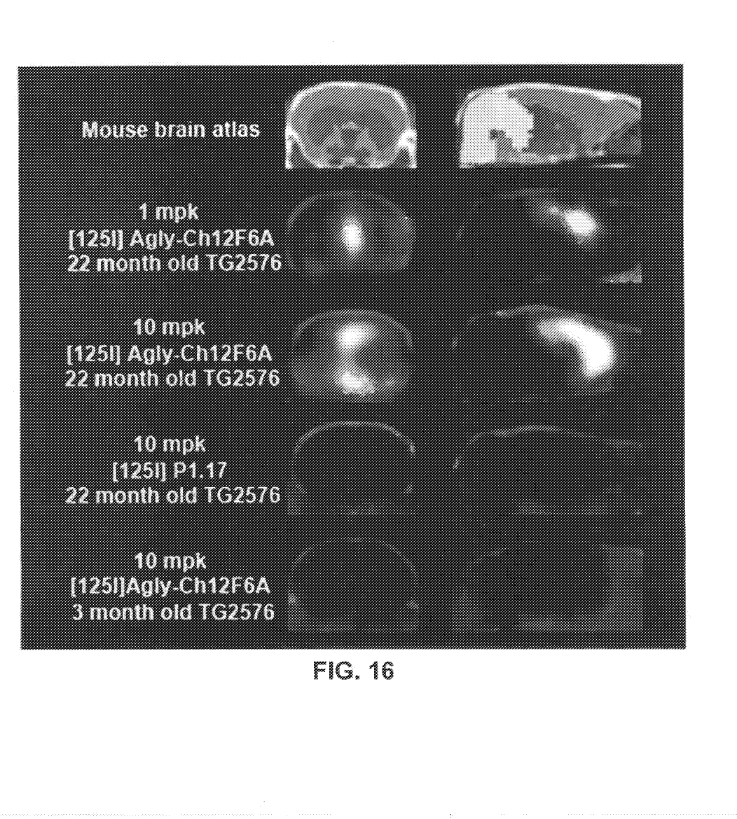

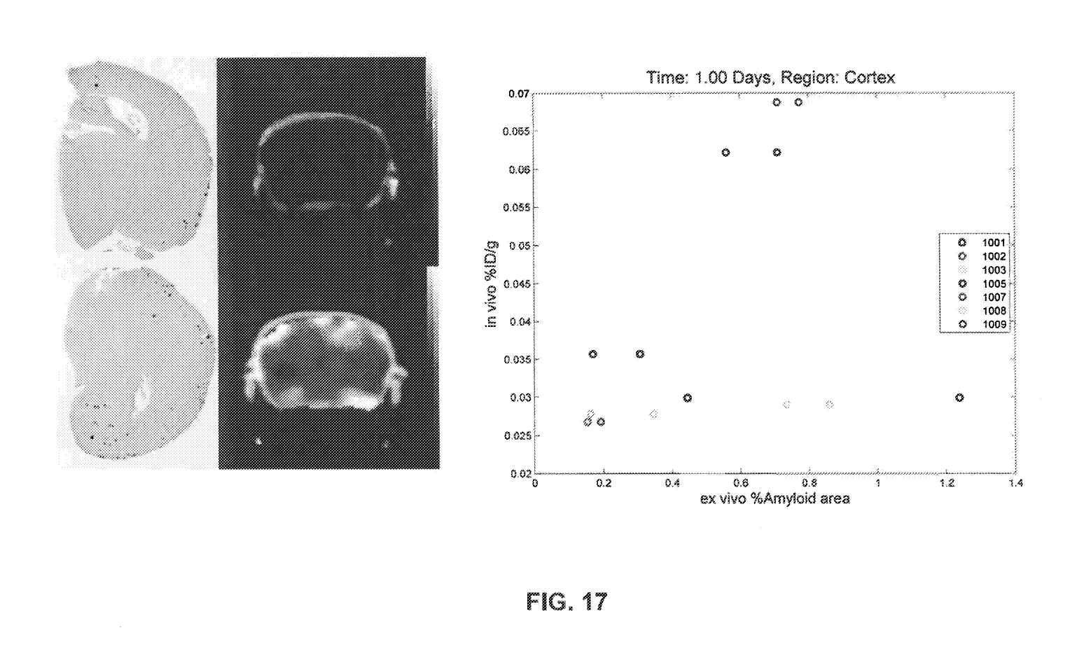

FIG. 17 shows that low plaque burden was successfully indicated in 3 of 3 animals and high plaque burden in 2 of 3 animals, One high plaque burden animal presented as a false negative (high plaque burden, low SPECT signal).

### Example 13

### Immunohistochemical and Histological Staining of [¹²⁵I]chBIIB037 in Tg2576 and WT Mouse Brain Vs. Mouse Brain Section Microautoradiography (mARG)

This example discloses immunohistochemical and histological staining of [¹²⁵I]chBIIB037 in Tg2576 and WT mouse brain as well as autoradiographical evaluation of brain tissues.

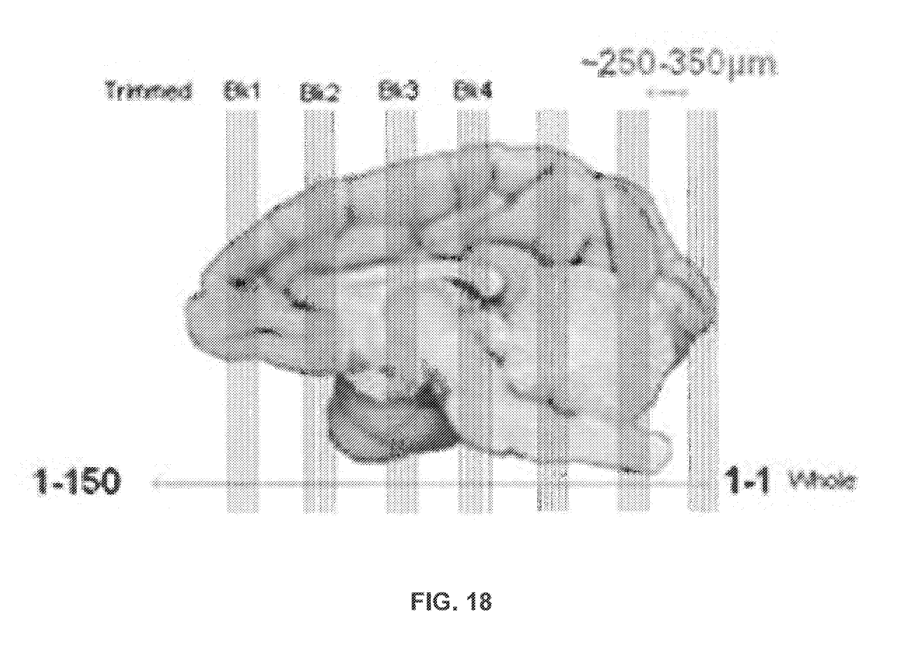

The protocol used for tissue sampling was provided by MPI Research Laboratories, Mattawan, Mich. Essentially, brains were received after dissection and immersed in 10% neutral buffered formalin. Fixed brains were then processed and embedded in paraffin. For brains processed whole, the tissue was oriented for coronal sectioning. For brains trimmed coronally, blocks were labeled numerically beginning with the anterior surface of the brain (e.g., block 1=forebrain, block 7=hindbrain). Either 5 and/or 10 μm consecutive sections were obtained from a minimum of 6 levels through the brain. A minimum of 1 section for IHC and 2 for ARG were obtained at each level. For Aβ IHC staining, one slide was stained from each level of brain (approximately every 250-500 μm). The specific thickness, number of sections, and distances between levels is documented in the study data (FIG. 18).

Immunohistochemistry (IHC) to define brain amyloid utilized Covance Mouse Anti-Human Beta Amyloid 1-16 Monoclonal Antibody (Clone 6E10, SIG-39320) as the primary antibody at a final concentration of 2 μg/ml. Biocare MM-Aβ Polymer was used as the secondary antibody with Vulcan Fast Red as the chromogen. Briefly, slides were immersed in 88% formic acid solution for 3 minutes, then rinsed in distilled water for 5 minutes, transferred to buffer and stained manually. An isotype (IgG1) negative control was run concurrently. Slides were counterstained with Richard Allen hematoxylin 2, cover-slipped and air dried overnight.

From the MPI Research Laboratories protocol, heads from frozen mouse carcasses were embedded in 2% carboxymethylcellulose (CMC) matrix and mounted on a microtome stage maintained at −20° C. Four quality control standards were placed into the frozen blocks prior to sectioning (for section thickness quality control). Cortical sections of 40 μm thickness were cut and captured on adhesive tape.

Brain section mARG was assessed by QPS (Delaware, Md.) under contract with MPI/inviCRO) for slides prepared from Tg2576 mice 101, 102 and 106 and WT mice 201 and 206. The brain of each mouse was harvested and frozen at the MPI Research facility in Mattawan, Mich. Frozen brains were shipped on dry ice and stored at −70° C. until sectioning. Coronal cross-sections were obtained from the six anatomic regions of each brain whenever possible to obtain sections encompassing nucleus accumbens, ventromedial hypothalamus, dorsomedial hypothalamus, dorsal raphe, nucleus of the solitary tract, and area postrema. An IHC staining procedure to localize brain Aβ in mARG sections was performed and the results were evaluated to see if co-localization of the Aβ and the radiolabeled test article was possible.

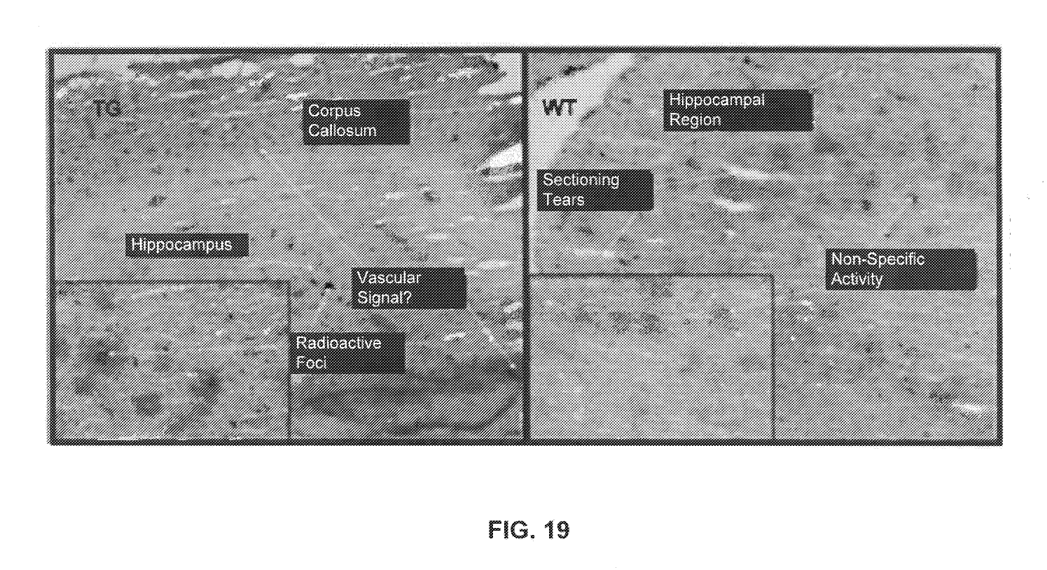

Two Tg and two WT mice were sacrificed at day 28 (n=2 Tg, n=1 WT) or day 7 (n=1 WT) to determine ex vivo whether radioactivity was localized at the target (amyloid plaques) in the relevant brain region(s). Representative data shows the presence of radioactive foci in hippocampal layers of Tg mouse brain compared to non-specific activity in a similar region taken from WT mouse (FIG. 19).

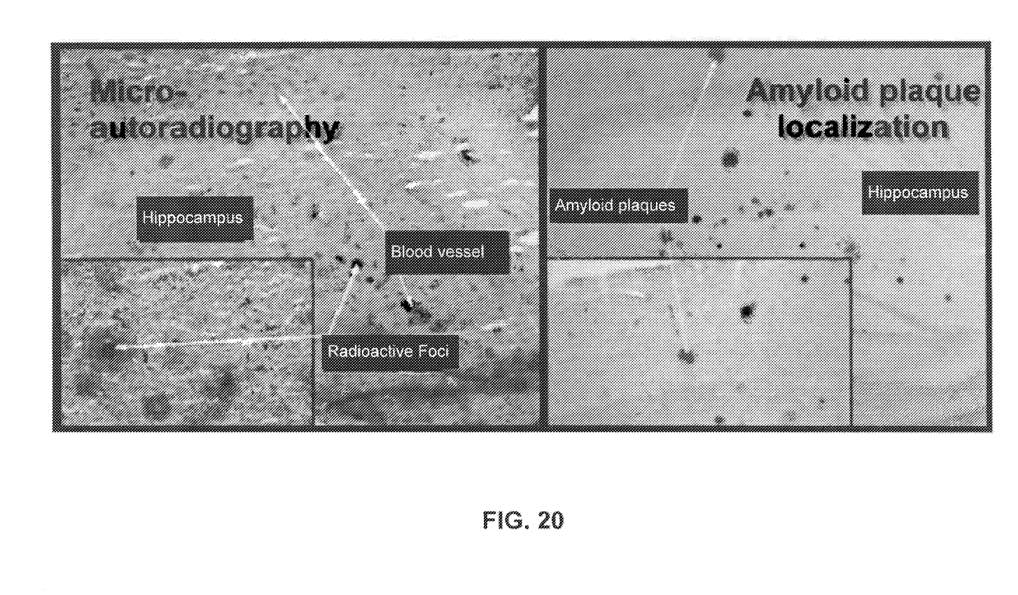

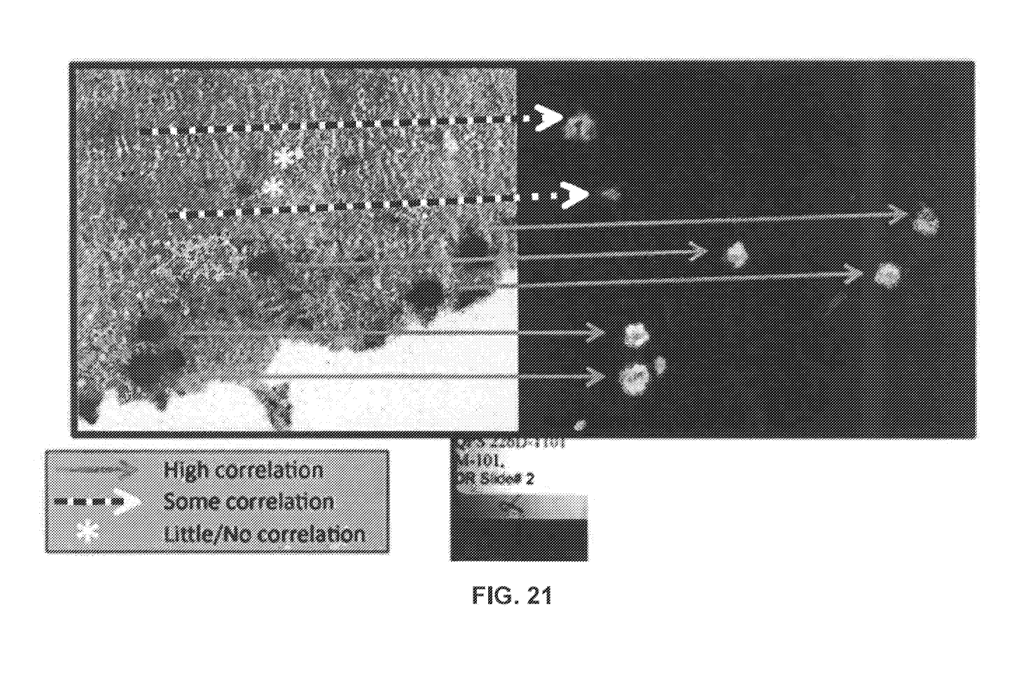

FIG. 20 shows co-localization of 6E10-derived Aβ immunostaining and mARG-derived radioactivity in adjacent sections of Tg2576 brain. Moreover, Thioflavin-S staining for Aβ in adjacent section in the area of the hippocampus using mARG sections showed equivocal results (FIG. 21).

### Example 14

### Biodistribution Studies Using [¹²⁵I]chBIIB037 and [¹²⁵I] Human BIIB037 F(Ab′)2′

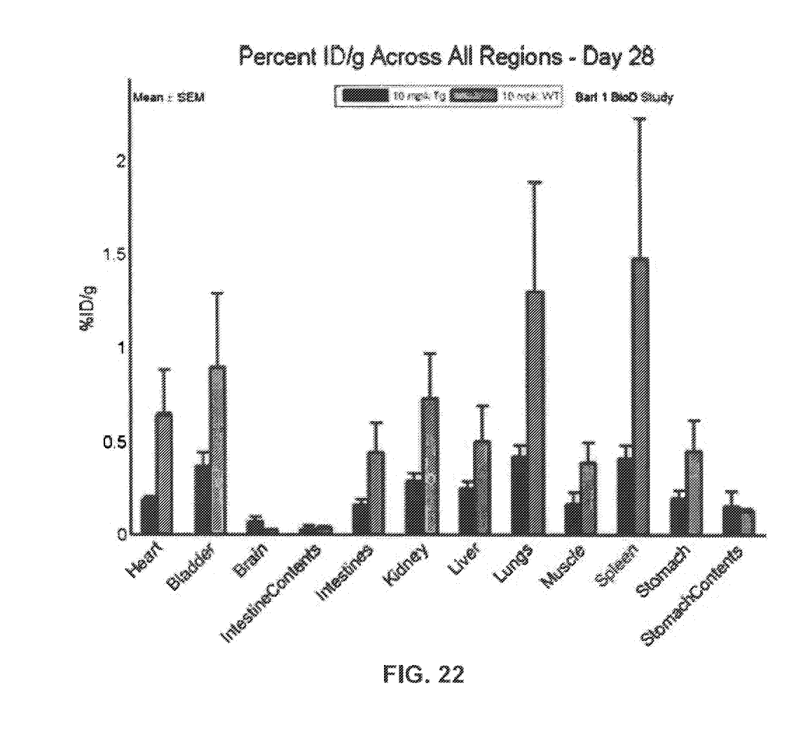

2 WT and 3 Tg mice were utilized at the end of the study (Table 5) to assess biodistribution of [¹²⁵I]chBIIB037 in several tissues. These animals were sacrificed at day 28 of the study 7 days following the last i.v. administration of radiolabeled antibody. Organs and tissues including spleen, whole brain, heart, kidney, muscle, liver, bladder, lungs stomach, intestine and blood were removed. Samples of organs and tissues were weighed and the radioactivity was measured using an automated gamma counter. The percent of injected dose (% ID) was calculated using a diluted standard solution and the decay-corrected injected dose. Radioactivity concentrations were thus expressed as percent of injected dose per gram of wet tissue (% ID/g) (Table 8 and FIG. 22).

TABLE 8
Biodistribution of [¹²⁵I] chBIIB037 in Tg2576 and WT mouse tissue

| | Tg | | WT | |
|---|---|---|---|---|
| | Mean % ID/g | SEM | Mean % ID/g | SEM |
| Heart | 0.192 | 0.016 | 0.644 | 0.234 |
| Bladder | 0.370 | 0.074 | 0.893 | 0.399 |
| Brain | 0.073 | 0.026 | 0.029 | 0.000 |
| Intestinal Contents | 0.033 | 0.017 | 0.037 | 0.010 |
| Intestines | 0.162 | 0.030 | 0.445 | 0.156 |
| Kidneys | 0.289 | 0.040 | 0.728 | 0.239 |
| Liver | 0.251 | 0.037 | 0.501 | 0.184 |
| Lungs | 0.423 | 0.057 | 1.301 | 0.583 |
| Muscle | 0.167 | 0.062 | 0.391 | 0.106 |
| Spleen | 0.411 | 0.067 | 1.471 | 0.744 |
| Stomach | 0.199 | 0.040 | 0.452 | 0.162 |
| Stomach Contents | 0.157 | 0.079 | 0.131 | 0.011 |

Table 8 and FIG. 22 include data expressed as % ID/g across tissues and show lowest but selective accumulation in brain tissue as indicated by the higher in vivo accumulation of radioactivity in Tg compared to WT mouse brain.

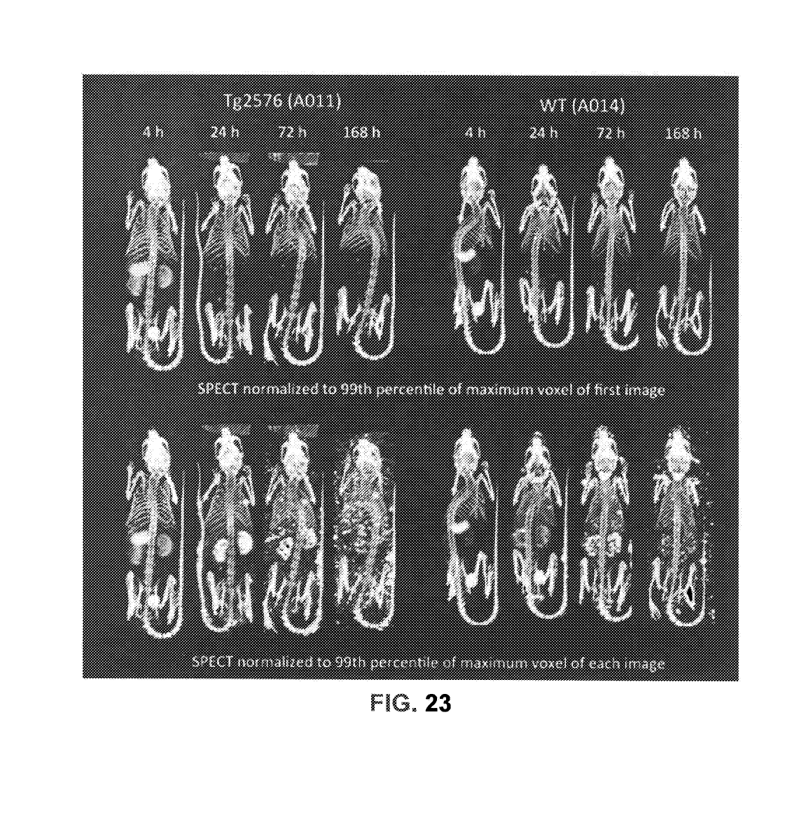

Biodistribution of [¹²⁵I]huBIIB037F(ab′)2 in non-target peripheral organs over the time course of the study was determined using ROI-analysis of those organs. FIG. 23 shows whole body SPECT images of in vivo [¹²⁵I]huBIIB037 F(ab′)2 in Tg2576 and WT mice. The whole body maximal intensity projections (MIPs) for animal 1011 (left) which showed the highest [¹²⁵I]huBIIB037F(ab′)2 in the brain is compared to that for WT mouse A1014 (right). Images are presented as two sets of MIPs in which data are normalized to the 99^{th} percentile of the maximal voxel of the first image (top) or are normalized to the 99^{th} percentile of the maximal voxel of each image (bottom).

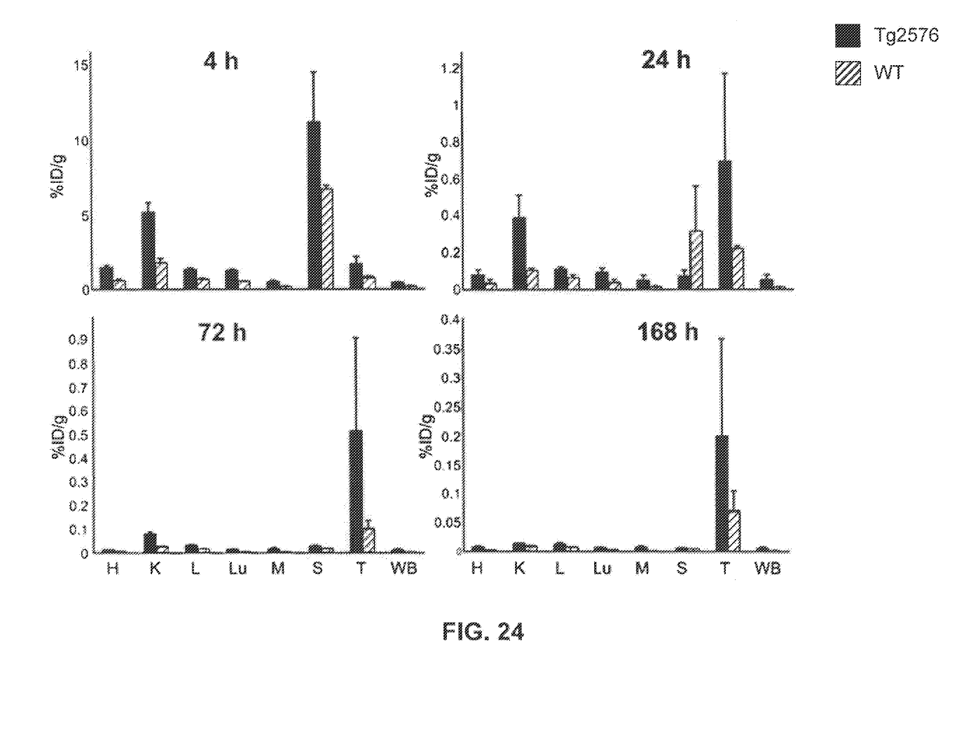

FIG. 24 shows quantitative whole body analysis of in vivo biodistribution of [¹²⁵I]huBIIB037 F(ab′)2 in non-target tissues of aged Tg2576 and young WT mice. Accumulation in thyroid tissue of Tg2576 mice is indicated by the higher in vivo accumulation of radioactivity (n=3) compared to WT (n=2) mice over time. Kidney accumulation decreases over time reflective of renal clearance of radiolabeled material.

### ***

The disclosure is not to be limited in scope by the specific embodiments described which are intended as single illustrations of individual aspects of the disclosure, and any compositions or methods which are functionally equivalent are within the scope of this disclosure indeed, various modifications of the disclosure in addition to those shown and described herein will become apparent to those skilled in the art from the foregoing description and accompanying drawings. Such modifications are intended to fall within the scope of the appended claims.

All publications and patent applications mentioned in this specification are herein incorporated by reference to the same extent as if each individual publication or patent application was specifically and individually indicated to be incorporated by reference.

1. A method of reducing brain amyloid plaques, comprising administering to a subject an anti-Aβ antibody or antigen-binding fragment thereof that
(a) binds to the same conformational epitope as BIIB037 antibody; or
(b) competitively inhibits BIIB037 binding to aggregated Aβ,
wherein the administration can reduce amyloid plaques in brain without substantially affecting vascular amyloid, and wherein BIIB037 antibody binds to an epitope comprising amino acids 3-6 of Aβ, and wherein the antibody is not BIIB037.

2. (canceled)

3. A method of minimizing the occurrence of microhemorrhage during chronic dosing of an anti-Aβ antibody or antigen-binding fragment thereof, comprising administering to a subject an anti-Aβ antibody that
(a) binds to amino acids 3-6 of Aβ; or
(b) competitively inhibits BIIB037 binding to aggregated Aβ, and wherein the antibody is not BIIB037.

4. (canceled)

5. A method of measuring the amount of brain amyloid plaques in a test subject, comprising:
(a) measuring the signal generated in the brain of a test subject following administration of an anti-Aβ antibody or antigen-binding fragment thereof that
(i) binds to the same conformational epitope as the BIIB037 antibody or
(ii) competitively inhibit BIIB037 antibody to aggregated Aβ,
wherein the antibody or antigen-binding fragment thereof is labeled with an agent that generates a measurable signal; and
(b) comparing the signal generated in the test subject to a signal generated upon administration of the labeled antibody or antigen-binding fragment thereof to one or more control subjects; wherein an increase in the signal generated in the test subject relative to the control subject correlates with an increase in brain amyloid plaques.

6. A method of treating a neurodegenerative disease characterized by brain amyloid plaques in a patient in need of treatment, comprising:
(a) administering an anti-Aβ antibody or antigen-binding fragment thereof that binds to the same conformational epitope as the BIIB037 antibody or that competitively inhibits BIIB037 antibody to aggregated Aβ, to a patient in need of neurodegenerative disease treatment, wherein the antibody or antigen-binding fragment thereof is labeled with an agent that generates a measurable signal;
(b) assessing the disease state in the patient upon review of a comparison of the signal measured in the patient to the signal measured following administration of the labeled antibody or antigen-binding fragment thereof to one or more control subjects; wherein an increase in the signal generated in the patient relative to the control subject correlates with an increase in brain amyloid plaques; and
(c) treating the patient with a therapy appropriate for the patient's disease state.

7-9. (canceled)

10. A method of assessing disease progression in a patient being treated for a neurodegenerative disease characterized by brain amyloid plaques, comprising:
(a) administering an anti-Aβ antibody or antigen-binding fragment thereof that binds to the same conformational epitope as the BIIB037 antibody or that competitively inhibits BIIB037 antibody to aggregated A to the patient in need of neurodegenerative disease treatment wherein the antibody or antigen-binding fragment thereof is labeled with an agent that generates a measurable signal, wherein the signal is measured in the patient following the administration;
(b) administering the labeled anti-Aβ antibody or antigen-binding fragment thereof at one or more time intervals following the administration of (a), wherein the signal is measured in the patient following the administration;
(c) assessing disease progression in the patient based on a change in the signal measured in the patient at the one or more time intervals following administration of (a); wherein an increase in the signal indicates progression of the neurodegenerative disease in the patient.

11-14. (canceled)

15. The method of claim 6, further comprising:
(d) administering the labeled anti-Aβ antibody or antigen-binding fragment thereof at one or more time intervals following the administration of (a), wherein the signal is measured in the patient following the administration; and
(e) assessing disease progression in the patient based on a change in the signal measured in the patient at the one or more time intervals following administration of (a); wherein an increase in the signal indicates progression of the neurodegenerative disease in the patient.

16. The method of claim 5, wherein the agent comprises one or more radioactive ligand(s).

17. The method of claim 16, wherein the ligand is ¹²⁵I.

18. The method of claim 5, wherein the signal generated by the agent is measured by single-photon emission computed tomography.

19-24. (canceled)

25. The method of claim 5, wherein VHCDR1, VHCDR2, and VHCDR3 of the anti-Aβ antibody or antigen-binding fragment thereof comprise the amino acid sequences of SEQ ID NOs: 3, 4, 5, respectively.

26. The method of claim 5, wherein VLCDR1, VLCDR2, and VLCDR3 of the anti-Aβ antibody or antigen-binding fragment thereof comprise the amino acid sequences of SEQ ID NOs: 6, 7, 8, respectively.

27. The method of claim 5, wherein the anti-Aβ antibody or antigen-binding fragment thereof comprises a VH and a VL, and wherein the VH comprises VHCDR1, VHCDR2, and VHCDR3 amino acid sequences of SEQ ID NOs: 3, 4, 5, and the VL, comprises VLCDR1, VLCDR2, and VLCDR3 amino acid sequences of SEQ ID NOs: 6, 7, 8.

28. The method of claim 5, wherein the anti-Aβ antibody or antigen-binding fragment thereof comprises a VH and a VL, and wherein the VH comprises SEQ ID NO: 1 and the VL comprises SEQ ID NO: 2.

29-43. (canceled)

44. The method of claim 10, wherein VHCDR1, VHCDR2, and VHCDR3 of the anti-Aβ antibody or antigen-binding fragment thereof comprise the amino acid sequences of SEQ ID NOs: 3, 4, 5, respectively.

45. The method of claim 10, wherein VLCDR1, VLCDR2, and VLCDR3 of the anti-Aβ antibody or antigen-binding fragment thereof comprise the amino acid sequences of SEQ ID NOs: 6, 7, 8, respectively.

46. The method of claim 10, wherein the anti-Aβ antibody or antigen-binding fragment thereof comprises a VH and a VL, and wherein the VH comprises VHCDR1, VHCDR2, and VHCDR3 amino acid sequences of SEQ ID NOs: 3, 4, 5, respectively and the VL, comprises VLCDR1, VLCDR2, and VLCDR3 amino acid sequences of SEQ ID NOs: 6, 7, 8, respectively.

47. The method of claim 10, wherein the anti-Aβ antibody or antigen-binding fragment thereof comprises a VH and a VL, and wherein the VH comprises SEQ ID NO: 1 and the VL comprises SEQ ID NO: 2.

48. The method of claim 6, wherein the agent comprises one or ore radioactive ligand(s).

49. The method of claim 10, wherein the agent comprises one or more radioactive ligand(s).
